(19) United States
(12) Patent Application Publication
(10) Pub. No.: US 2002/0068279 A1
(43) Pub. Date: Jun. 6, 2002

(54) Novel Proteins and nucleic acids encoding the same

(75) Inventors: Catherine Burgess, Wethersfield, CT (US); Sudhirdas K. Prayaga, O'Fallon, MO (US); Richard A. Shimkets, West Haven, CT (US); Luca Rastelli, Guilford, CT (US); Bryan Zerhusen, Branford, CT (US); Peter Mezes, Old Lyme, CT (US)

Correspondence Address: Ivor R. Elrifi, Ph.D., Mintz, Levin, Cohn, Ferris,, Glovsky and Popeo, P.C., One Financial Center, Boston, MA, 02111, US

(73) Assignee: CuraGen Corporation

(21) Appl. No.: 09/730,617

(22) Filed: Dec. 5, 2000

Related U.S. Application Data
(60) Provisional application No. 60/169,056, filed on Dec. 6, 1999.
(60) Provisional application No. 60/169,886, filed on Dec. 9, 1999.
(60) Provisional application No. 60/169,866, filed on Dec. 9, 1999.
(60) Provisional application No. 60/170,252, filed on Dec. 10, 1999.
(60) Provisional application No. 60/175,740, filed on Jan. 12, 2000.

(51) Int. Cl. C12Q 1/68; G01N 33/53; G01N 33/542; C07H 21/04; A61K 48/00; C07K 14/435

(52) U.S. Cl. 435/6; 435/7.9; 435/325; 435/320.1; 530/350; 536/23.5; 514/44; 530/388.1

(57) ABSTRACT

The invention provides novel human transmembrane (NOVTRAN), neuromedin (NOVNEUR), gonadtropin (NOVGON), and interleukin-1 receptor antagonist (NOVINTRA A and B) proteins (NOVX proteins) and isolated nucleic acid molecules encoding the same. Also provided are antibodies that immunospecifically bind to NOVX polypeptides or polynucleotides, or derivatives, variants, mutants, or fragments thereof. The invention additionally provides methods in which a NOVX polypeptide, polynucleotide, and antibody are used in the detection, prevention, and treatment of a broad range of pathological states. Also provided are methods of diagnosing and treating a lung disease associated with differential expression of human interleukin-1 epsilon.

### RELATED APPLICATIONS

This application claims priority to U.S. Ser. No. 60/169,056, filed Dec. 6, 1999; U.S. Ser. No. 60/169,886, filed Dec. 9, 1999; U.S. Ser. No. 60/169,866, filed Dec. 9, 1999; U.S. Ser. No. 60/170,252, filed Dec. 10, 1999; and U.S. Ser. No. 60/175,740, filed January 12, 2000; the teachings of which are incorporated by reference in their entirety.

### FIELD OF THE INVENTION

The invention relates to polynucleotides and the polypeptides encoded thereby.

### BACKGROUND OF THE INVENTION

### Transmembrane Proteins

Transmembrane proteins, as a class, have been implicated in signal transduction, control of cell adhesion, regulation of cell growth and proliferation (including development and oncogenesis), transport of ions or metabolites (e.g., ion channels), and motility (including the ability to suppress metastatic potential). Expression of many transmembrane proteins has been shown to be associated with a variety of tumors.

Generally, transmembrane proteins are divided into two groups, extrinsic or intrinsic (integral) membrane proteins, based upon the ease with which the proteins can be removed from the membrane. The majority of known integral membrane proteins are transmembrane proteins, which comprise an extracellular, a transmembrane, and an intracellular domain. Transmembrane proteins are typically embedded into the cell membrane by one or more regions comprising 15 to 25 hydrophobic amino acids, which are predicted to adopt an alpha-helical structure. There remains a need for the identification of novel transmembrane proteins, which are involved in cell-signaling pathways implicated in cell adhesion, cell proliferation, and metabolite transport processes.

### Neuromedins

The bombesin-like peptides comprise a large family of peptides initially isolated from frog skin, and later found to be widely distributed in mammalian neural and endocrine cells. Gastrin-releasing peptide ((GRP); acc: 137260) was the first mammalian bombesin-like peptide to be characterized. In amphibians, the bombesin-like peptides have been classified into 3 subfamilies: the bombesins, the ranatensins, and the phylloitorins. The amidated decapeptide neuromedin B (NMB) is the mammalian homolog of the amphibian bombesin-like peptide ranatensin, and is similar to bombesin, as well as GRP, at both the sequence level, and structurally.

NMB, like other mamalian bombesin-like peptides, is widely distributed in the central nervous system and gastrointestinal tract, and is a potent mitogen and growth factor for normal and neoplastic lung, and for gastrointestinal epithelial tissue. These peptides bind to G protein-coupled receptors (e.g. GRP receptor, neuromedin B receptor, bombesin receptor subtype-3 (BRS3)) on the cell surface to elicit their effects, including modulation of smooth-muscle contraction, exocrine and endocrine processes, metabolism, and behavior. Accordingly, there remains a need for the identification of novel neuromedins, which are involved in modulation of these diverse procesesses.

### Gonadotropins

Human chorionic gonadotropin (hCG) belongs to a family of glycoprotein hormones, including luteinizing hormone (lutropin, hLH), follitropin (FSH), and thyrotropin (TSH). These proteins are involved in reproductive development and reproductive cycle maintainence, as well as hormone-modulated cell growth processes. Many cancers secrete hormones such as hCG and/or an hCG subunit. Indeed, elevated hCG serum concentration is considered a reliable indicator of the presence of some tumors.

Gonadotropin-releasing hormone (GnRH) agonists and antagonists have proven effective in the treatment of certain conditions which require inhibition of LH/FSH release. In particular, GnRH-based therapies have proven effective in the treatment of endometriosis, uterine fibroids, polycystic ovarian disease, precocious puberty and several gonadal steroid-dependent neoplasia, most notably cancers of the prostate, breast and ovary. GnRH agonists and antagonists have also been utilized in various assisted fertilization techniques and have been investigated as a potential contraceptive in both men and women. They have also shown possible utility in the treatment of pituitary gonadotrophe adenomas, sleep disorders such as sleep apnea, irritable bowel syndrome, premenstrual syndrome, benign prostatic hyperplasia, hirsutism, as an adjunct to growth hormone therapy in growth hormone deficient children, and in murine models of lupus. Accordingly, there remains a need for the identification of novel gonadotropin-like proteins, and modulators of the same, which are involved in hormonally-regulated disease processes.

### Interleukin Receptors

Interleukin-1 is a cytokine with a wide range of biological and physiological effects, including fever, prostaglandin synthesis (in e.g., fibroblasts, muscle and endothelial cells), T-lymphocyte activation, and interleukin 2 production. The IL-1 receptor antagonist (IL1RN or IL-1RA) is a protein that binds to IL-1 receptors and inhibits the binding of IL1-alpha and IL1-beta. As a consequence, the biologic activity of these 2 cytokines is neutralized in physiologic and pathophysiologic immune and inflammatory responses. IL1RN is the first-to-be described, naturally occurring cytokine or hormone-like molecule that functions as a specific receptor antagonist. The gene for interleukin-1 receptor antagonist protein has been cloned, and the expression of the gene, as well as the biologic characteristics of the protein, which was referred to as interleukin-1-receptor antagonist protein (IRAP), studied. It has been shown that IL1RN specifically inhibited IL-1 bioactivity on T cells and endothelial cells in vitro, and was a potent inhibitor of IL-1 induced corticosterone production in vivo.

IL1RN levels are elevated in the blood of patients having a variety of infectious, immune, and traumatic conditions. Therefore, there remains a need for the identification of additional antagonists of interleukin-1 receptor, which are involved in modulation of immune and inflammatory responses.

### SUMMARY OF THE INVENTION

The invention is based in part on the discovery of novel nucleic acids encoding a novel human transmembrane protein (NOVTRAN), a neuromedin peptide (NOVNEUR), a gonadotropin-like protein (NOVGON), and two interleukin-1 receptor antagonist proteins (NOVINTRA A and B), hereinafter collectively referred to as “NOVX” polypeptides or nucleic acids.

In one aspect, the invention provides isolated nucleic acid sequences encoding novel NOVTRAN, NOVNEUR, NOVGON, NOVINTRA A, and NOVINTRA B polypeptides, wherein the nucleic acid sequences is selected from SEQ ID NOs: 1, 3, 5, 7, 9, and 11, respectively, or an allelic or substitution variant thereof. In another aspect, there is provided an oligonucleotide that includes a portion of a NOVX nucleic acid seqeunce, e.g. SEQ ID NOs: 1, 3, 5, 7, 9, and 11, respectively.

In other aspects, the invention provides a vector comprising one or more of the isolated nucleic acid sequences or oligonucleotides described herein, and a host cell transformed with one or more vectors described herein. Also provided is a method for producing a NOVX polypeptide by culturing a host cell transformed with one or more vectors described herein under conditions suitable for the expression of the NOVX protein encoded by the vector.

In yet another aspect, the invention provides an antibody that binds specifically to a NOVX nucleic acid or oligonucleotide described herein. The antibody can be a monoclonal or polyclonal antibody, or fragments and derivatives thereof, e.g. a labeled antibody.

In still another aspect, the invention provides a pharmaceutical composition that comprises an isolated nucleic acid or oligonucleotide described herein and a pharmaceutically-acceptable carrier or excipient.

In another aspect, there is provided an isolated NOVTRAN, NOVNEUR, NOVGON, NOVINTRA A, and NOVINTRA B polypeptide encoded by an isolated nucleic acid sequence or oligonucleotide described herein. In some aspects, the isolated NOVX protein comprises an amino acid sequence selected from SEQ ID NOs: 2, 4, 6, 8, 10, or 12, respectively, or functional variants or fragments thereof. In another embodiment, a variant or fragment of a NOVX protein retains the respective NOVX-like protein activity.

In yet another aspect, there is provided an antibody that binds specifically to an isolated NOVX protein, or fragment thereof. The antibody can be a monoclonal or polyclonal antibody, or fragments and derivatives thereof, e.g. a labeled antibody.

In still another aspect, the invention provides a pharmaceutical composition that comprises an isolated NOVX protein, or a fragment thereof, and a pharmaceutically-acceptable carrier or excipient.

The invention further provides a method of treating a disorder in a mammal by administering at least one agent which modulates the expression or activity of a NOVX protein. In one embodiment, the protein is NOVTRAN and the disorder is disease involving altered cell signaling, such as cancer, immune and hematopoietic disorders, or neurodegenerative disease. In another embodiment, the protein is NOVNEUR, and the disorders is an endocrine, muscle, or neurologic diseases, or cancer (e.g. central nervous system, lung, breast, colon, ovarian, kidney, or thyroid cancer. In another embodiment, the protein is NOVGON, and the disorder involves reproductive development, weight gain/loss, metabolic function, or other hormonally-modulated diseases, such as cancer (e.g. melanoma). In yet another embodiment, the protein is a NOVINTRA protein, and the disorder involves bone metabolism and structure, inflammatory response, and immune regulation, or diseases such as septic shock, stroke, diabetes, arthritis and cancer. Accordingly, NOVX nucleic acids and proteins may be useful in the prevention and/or treatment of these diseases, as well as the identification of modulators of these diseases.

The invention is also based, in part, on the discovery that human interleuking-1 epsilon (also identified herein as NOVINTRA C) is overexpressed in lung cancer tissue and differentially expressed in small airway epithelium. Thus, in one embodiment, the invention provides methods for determining the presence of or predisposition to lung disease associated with differential expression of human IL-1 epsilon polypeptide, or nucleic acid, by measuring the expression of the protein, or nucleic acid, in a subject and comparing the expression level to that of a control which does not have the disease. In another embodiment, the invention provides a method of treating a lung disease in a mammal by administering to the mammal at least one agent which modulates the expression or activity of a human IL-1 epsilon protein. In some embodiments, the lung disease is lung cancer, asthma, emphysema, allergic lung irritation, or lung inflammation.

The invention further provides methods of identifying a NOVX protein or nucleic acid encoding the same in a sample by contacting the sample with a compound that specifically binds to the polypeptide or nucleic acid, e.g. an antibody, and detecting complex formation, if present. Also provided are methods of identifying a compound that modulates the activity of a NOVX protein by contacting the protein with a compound and determining whether the NOVX protein activity is modified.

In yet another aspect, the invention provides a method of determining the presence of or predisposition of a NOVX protein-associated disorder in a subject, comprising the step of providing a sample from the subject and measuring the amount of NOVX protein in the subject sample. The amount of the particular protein in the subject sample is then compared to the amount of that protein in a control sample. A control sample is preferably taken from a matched individual, i.e., an individual of similar age, sex, or other general condition but who is not suspected of having a NOVX protein-associated condition. Alternatively, the control sample may be taken from the subject at a time when the subject is not suspected of having a NOVX protein-associated disorder. In some embodiments, the particular protein of interest is detected using a specific antibody, as described above.

In a further embodiment, the invention provides a method of determining the presence of or predisposition of a NOVX protein-associated disorder in a subject. The method includes providing a nucleic acid sample, e.g., RNA or DNA, or both, from the subject and measuring the amount of the respective protein-encoding nucleic acid in the subject nucleic acid sample. The amount of NOVX protein-encoding nucleic acid in the subject nucleic acid is then compared to the amount of such nucleic acid in a control sample. An alteration in the amount of the particular protein-encoding nucleic acid in the sample relative to the amount of such nucleic acid in the control sample indicates the subject has a NOVX protein-associated disorder.

In still another aspect, there is provided a method of treating or preventing or delaying a NOVX protein-associated disorder. The method comprises administering to a subject in which such treatment or prevention or delay is desired a nucleic acid encoding a NOVX protein, or an antibody specific for either, in an amount sufficient to treat, prevent, or delay the particular protein-associated disorder in the subject.

Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1A depicts the NOVTRAN nucleic acid sequence of the invention (SEQ ID NO: 1); start and stop codons for the coding sequence are shown in bold.

FIG. 1B depicts the NOVTRAN amino acid sequence of the invention (SEQ ID NO: 2).

FIG. 2A depicts the BlastN identity search for the NOVTRAN nucleic acid sequence of the invention.

FIG. 2B depicts the BlastX identity search for the NOVTRAN nucleic acid sequence of the invention.

FIG. 3A depicts the NOVNEUR nucleic acid sequence of the invention (SEQ ID NO: 3); start and stop codons for the coding sequence are shown in bold and the putative UTRs are underlined.

FIG. 3B depicts the NOVNEUR amino acid sequence of the invention (SEQ ID NO: 4).

FIG. 4A depicts the BlastN identity search for the NOVNEUR nucleic acid sequence of the invention.

FIG. 4B depicts the BlastX identity search for the NOVNEUR amino acid sequence of the invention.

FIG. 5 depicts the ClustalW sequence alignment for the NOVNEUR polypeptide of the invention.

FIG. 6A depicts the NOVGON nucleic acid sequence of the invention (SEQ ID NO: 5)

FIG. 6B depicts the NOVGON amino acid sequence of the invention (SEQ ID NO: 6)

FIG. 7A depicts the BlastN identity search for the NOVGON nucleic acid sequence of the invention.

FIG. 7B depicts the BlastX identity search for the NOVGON amino acid sequence of the invention.

FIG. 8 depicts the ClustalW sequence alignment for the NOVGON polypeptide of the invention.

FIG. 9A depicts the NOVINTRA A nucleic acid sequence of the invention (SEQ ID NO: 7). The stop codon is shown in bold and the putative UTR is underlined.

FIG. 9B depicts the NOVINTRA A amino acid sequence of the invention (SEQ ID NO: 8)

FIG. 10A depicts the BlastN identity search for the NOVINTRA A nucleic acid sequence of the invention.

FIG. 10B depicts the BlastX identity search for the NOVINTRA A amino acid sequence of the invention.

FIG. 11 depicts the ClustalW sequence alignment for the NOVINTRA A polypeptide of the invention.

FIG. 12A depicts the NOVINTRA B nucleic acid sequence of the invention (SEQ ID NO: 9). The start/stop codons are shown in bold and the putative UTR is underlined.

FIG. 12B depicts the NOVINTRA B amino acid sequence of the invention (SEQ ID NO: 10).

FIG. 13A depicts the BlastN identity search for the NOVINTRA B nucleic acid sequence of the invention.

FIG. 13B depicts the BlastX identity search for the NOVINTRA B amino acid sequence of the invention.

FIG. 14 depicts the ClustalW sequence alignment for the NOVINTRA B polypeptide of the invention.

FIG. 15A depicts the NOVINTRA C nucleic acid sequence of the invention (one of SEQ ID NO: 11).

FIG. 15B depicts the NOVINTRA C amino acid sequence of the invention (SEQ ID NO: 12).

FIG. 16A depicts the BlastN identity search for the NOVINTRA C nucleic acid sequence of the invention.

FIG. 16B depicts the BlastX identity search for the NOVINTRA C amino acid sequence of the invention.

FIG. 17 depicts the ClustalW sequence alignment for the NOVINTRA C polypeptide of the invention.

FIG. 18 is a hydrophobicity plot for the NOVTRAN protein of the invention.

FIG. 19 is a hydrophobicity plot for the NOVNEUR protein of the invention.

FIG. 20 is a hydrophobicity plot for the NOVGON protein of the invention.

FIG. 21 is a hydrophobicity plot for the NOVINTRA A protein of the invention.

FIG. 22 is a hydrophobicity plot for the NOVINTRA B protein of the invention.

FIG. 23 is a hydrophobicity plot for the NOVINTRA C protein of the invention.

### DETAILED DESCRIPTION OF THE INVENTION

The invention is based in part on the discovery of novel NOVX nucleic acids encoding polypeptides that are homologous to previously described transmembrane, neuromedin, gonadotropin, and interleukin-1 receptor antagonist proteins. The new genes and proteins are named NOVTRAN, NOVNEUR, NOVGON, NOVINTRA A, NOVINTRA B, and NOVINTRA C (human IL-1 epsilon), respectively.

### NOVTRAN

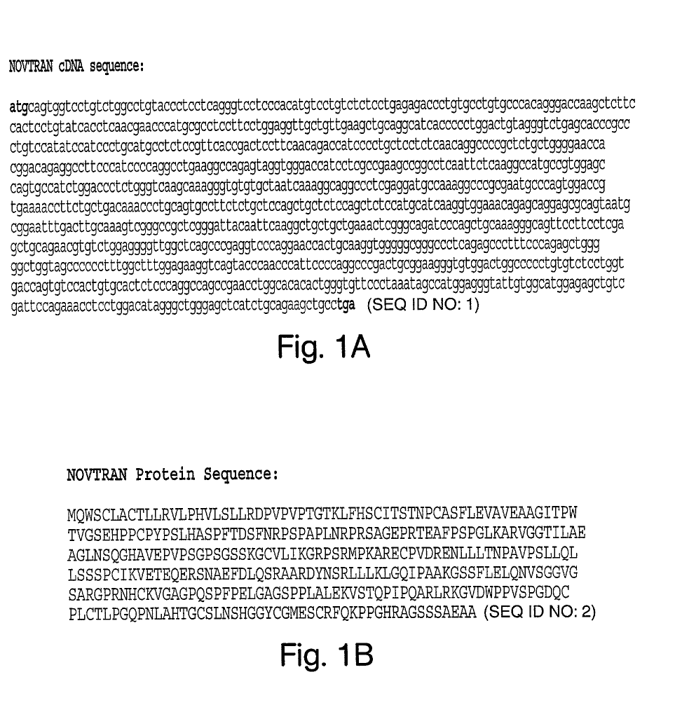

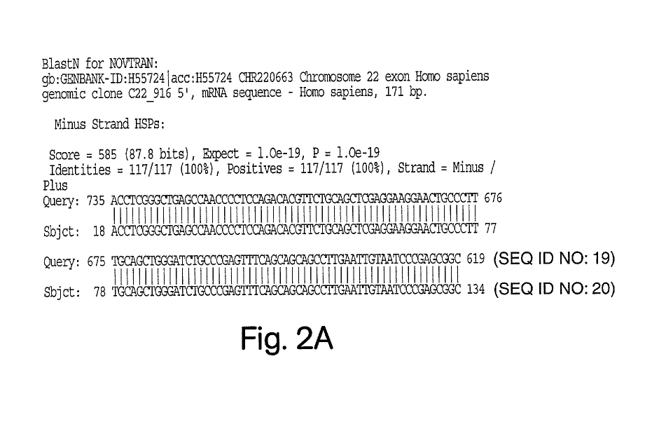

A novel human transmembrane protein (NOVTRAN) gene was identified based on its homology to human chromsome 22 exon mRNA (acc: H55724) (see FIG. 2A). Protein sorting prediction analysis (PSORT) revealed that this sequence localized to the plasma membrane protein (certainty=0.6500), the mitochondrial inner membrane (certainty=0.5638), mitochondrial matrix (certainty=0.3572) and/or intermembrane space (certainty=0.3572). Sequence analysis of the genomic DNA fragment AC00763_A generated an extended, predicted cDNA of 1047 nucleotides (FIG. 1A; start/stop codons shown in bold). The amino acid sequence encoded by the cDNA is not similar to any known proteins (see FIGS. 1B and 2B). The NOVTRAN nucleic acid sequence of the invention is shown in FIG. 1A. The disclosed nucleotide sequence encodes a NOVTRAN protein of 348 amino acids (SEQ ID NO: 2; also shown in FIG. 1B). This sequence contains a likely signal peptide cleavage site between amino acids residues 19 and 20 (VLS-LL) of SEQ ID NO: 1B.

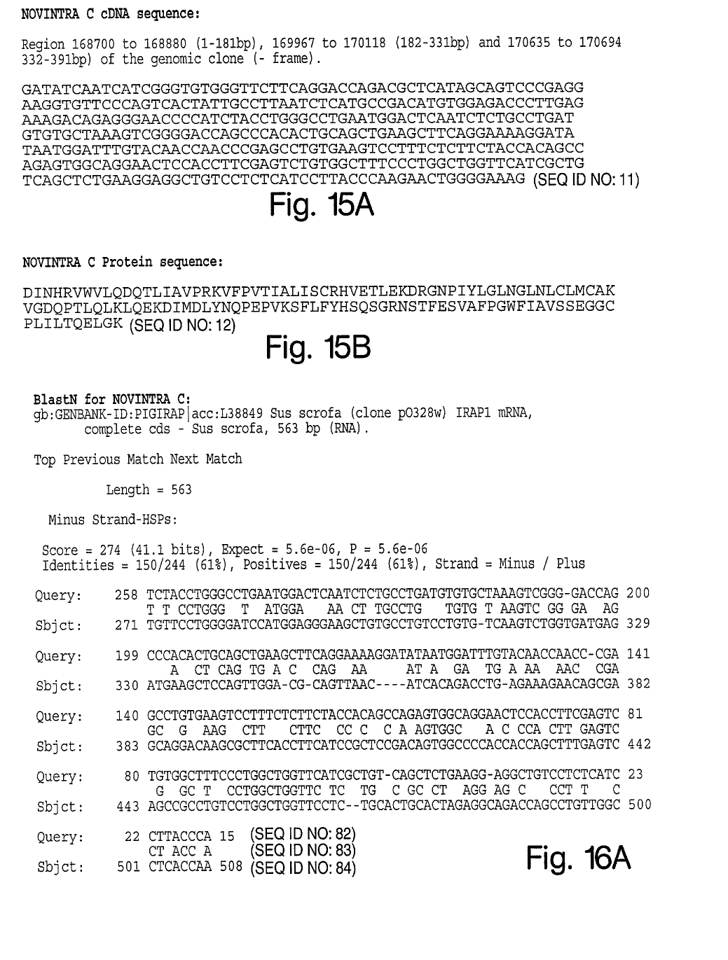

The NOVTRAN protein disclosed is not similar to any known proteins (see FIG. 2B). Hydrophobicity analysis of the amino acid sequence indicates that NOVTRAN is a largely hydrophilic, having a distinct hydrophobic domain at its N-terminus. See FIG. 18.

A NOVTRAN polypeptide of the invention encompasses a protein described herein, or mature forms arising therefrom as a result of post-translational modifications. Thus, the proteins of the invention encompass both a precursor and any active forms of the NOVTRAN protein.

### NOVNEUR

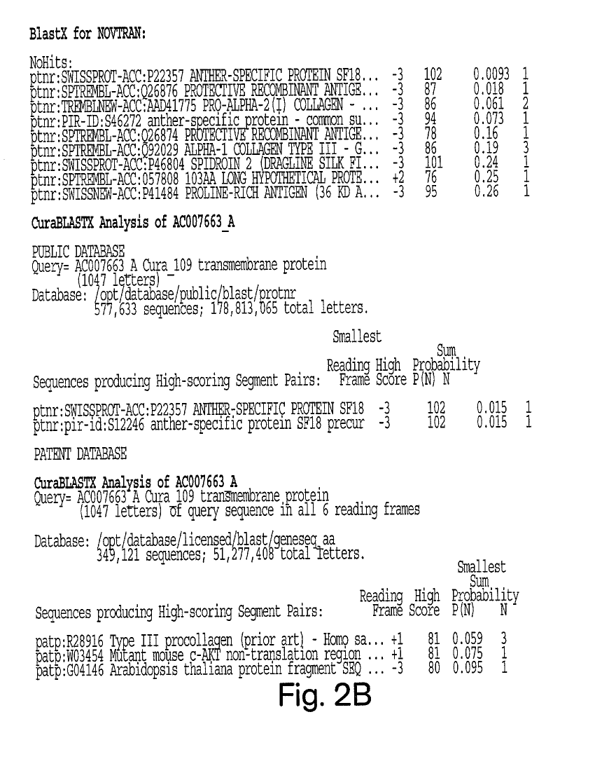

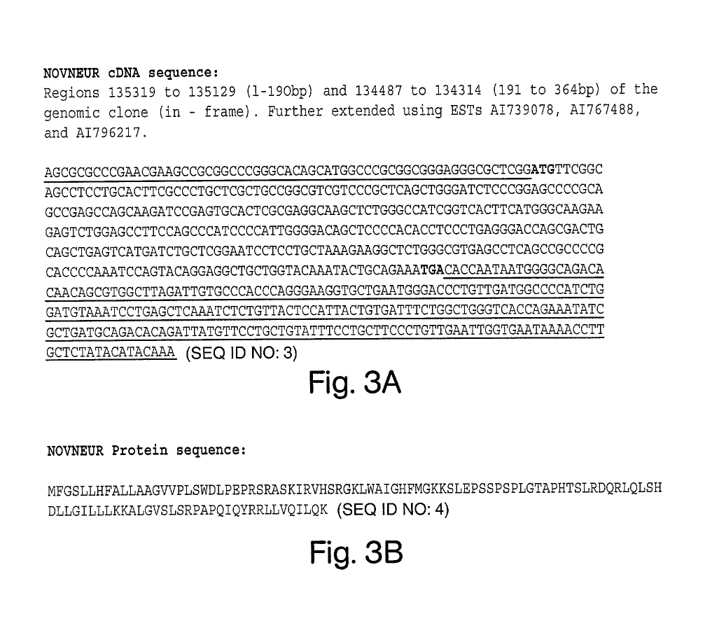

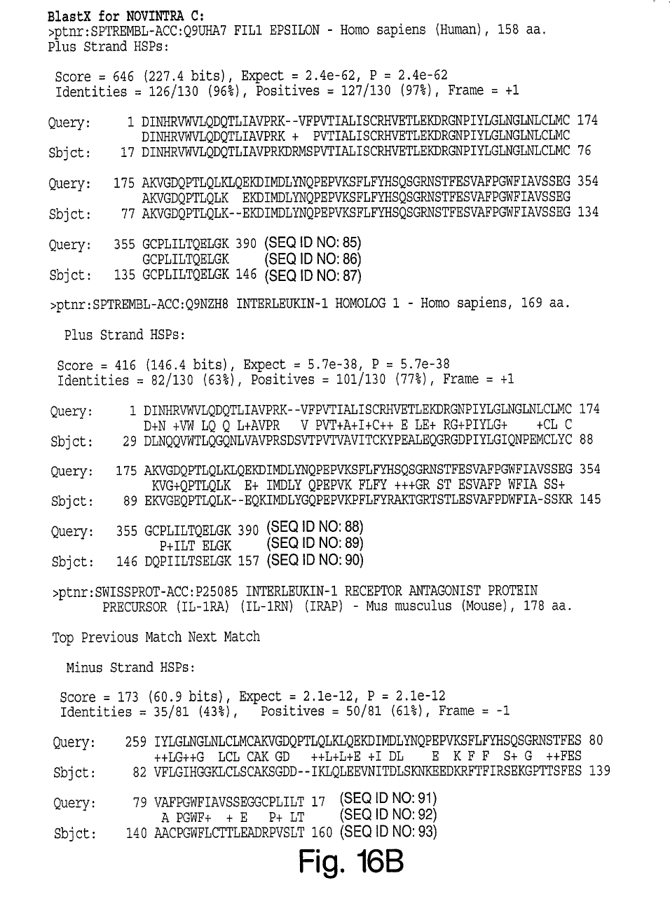

A novel human neuromedin (NOVNEUR) gene was identified based on its homology to mammalian neuromedin B mRNA (NMB) (acc: M21551) (see FIG. 4A). Sequence analysis of the genomic DNA fragment AC016771_A generated a cDNA of 646 nucleotides (FIG. 3A) containing a 336 nucleotide coding sequence. The amino acid sequence encoded by the cDNA is 88% identical and 88% similar to a 121 amino acid human neuromedin B-32 precursor (SwissProt acc: P08949) (see FIGS. 3B and 4B). PSORT analysis confirmed this sequence is a cytoplasmic protein (certainty=0.6138). The NOVNEUR nucleic acid sequence of the invention is shown in FIG. 3A. The disclosed sequence is 646 nucleotides in length (SEQ ID NO: 3), and encodes a NOVNEUR protein of 112 amino acids (SEQ ID NO: 4; also shown in FIG. 3B). Hydrophobicity analysis of the amino acid sequence indicates that NOVNEUR contains two distinct hydrophobic domains at its N-terminus, and near its C-terminus. See FIG. 19.

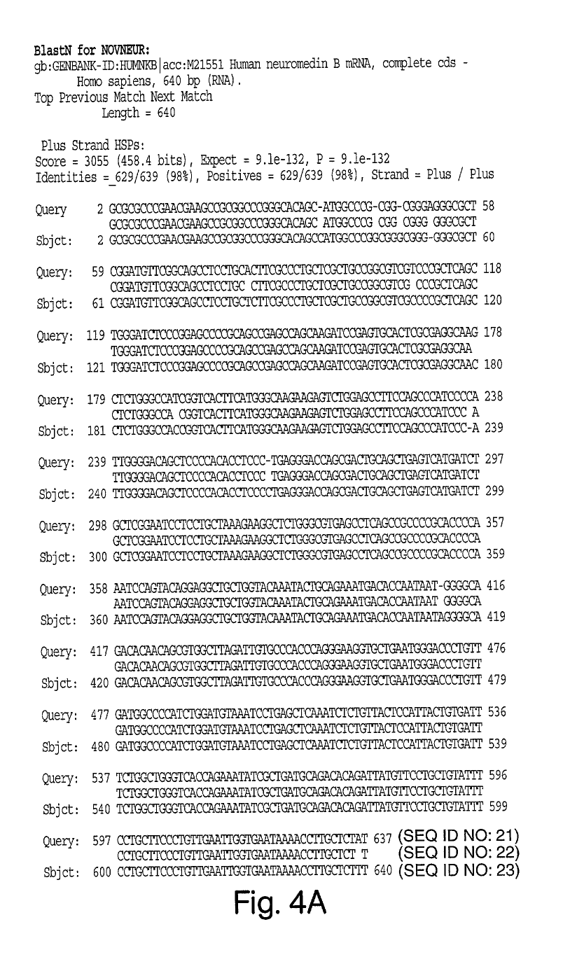

The NOVNEUR protein disclosed has substantial homology to both human neuromedin B-32 precursor and rat neuromedin B precursor (see FIG. 5). NOVNEUR is 88% identical to human neuromedin B precursor at the amino acid level, over residues −6 to 112 of of SEQ ID NO: 4. NOVNEUR shares the bombesin-like peptide family consensus sequence, W-A-x-G-[SH]-[LF]-M (where positions 5 and 6 are His-Phe in NMB, ranatensin, and NOVNEUR (see residues 40-46 of SEQ ID NO: 4) shared by all putative members of this family (see PFAM database at www.sanger.ac.uk). The NOVNEUR polypeptide of the invention is more identical (88%) to human NMB at the amino acid level than the nearest family member, R. Norvegicus (rat) NMB precursor (71% over 114 amino acids) (see UniGene database, www.ncbi.nih.gov/UniGene).

A NOVNEUR polypeptide of the invention encompasses a protein described herein, or mature forms arising therefrom as a result of post-translational modifications. Thus, the proteins of the invention encompass both a precursor and any active forms of the NOVNEUR protein.

As discussed in more detail in Example 1, below, NOVNEUR is expressed in several normal cell and tissue lines, and in several cancer cell lines, including central nervous system (CNS) cancer, lung cancer (non small cell), breast cancer, colon cancer and ovarian cancer. In addition, in comparison to surgical normal adjacent tissue, the clone is expressed in kidney cancer (clear cell type), prostate cancer, kidney cancer and thyroid cancer, as well as in lung cancer and kidney cancer. These results suggest that NOVNEUR may be used as a specific diagnostic probe for several types of cancer, and that the NOVNEUR protein may serve as a target for an antibody or for a small molecule drug in the treatment of several cancers, among other utilities, as describe herein.

### NOVGON

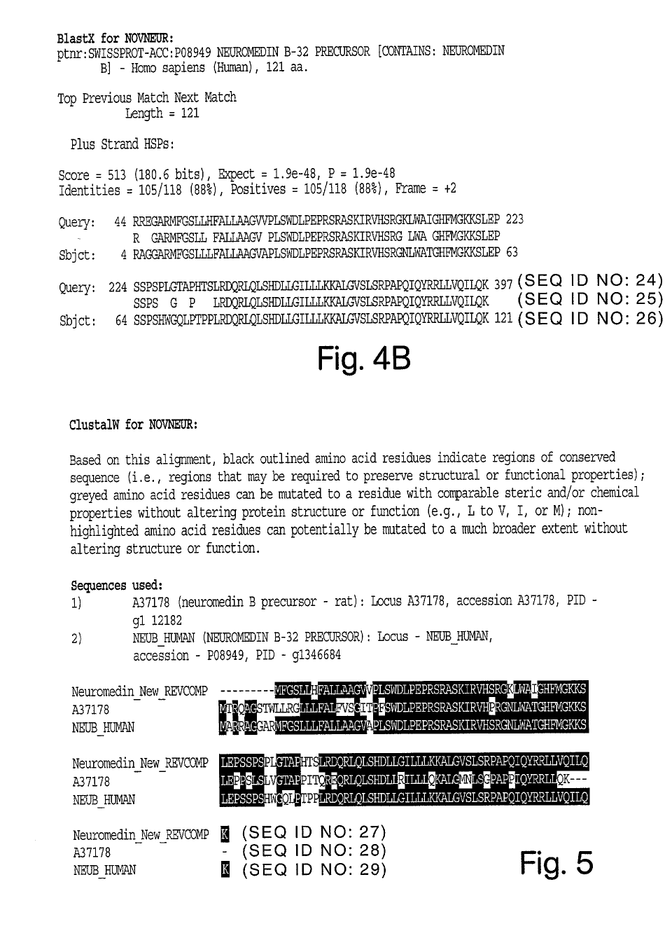

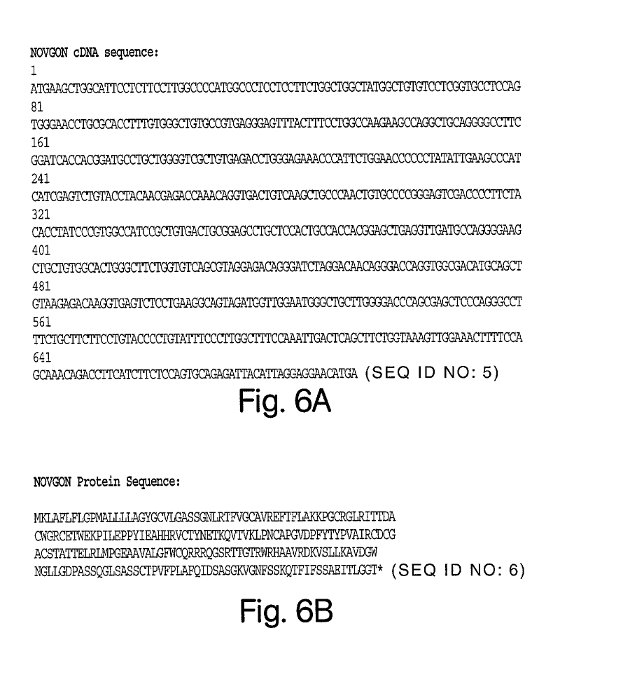

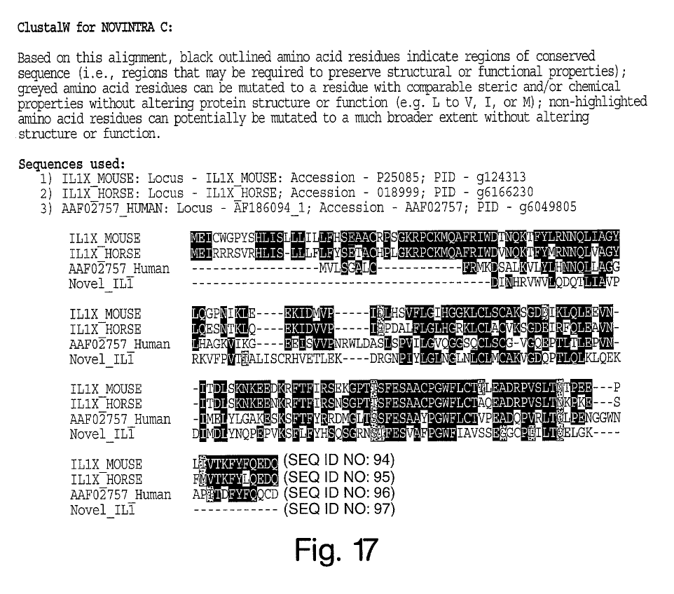

A novel human gonadotropin-like (NOVGON) gene was identified based on its homology to Salmo salar gonadotropin II beta subunit mRNA (GII-B) (acc: AF146151) (see FIG. 7A). Sequence analysis of the genomic DNA fragment AL049871 generated a cDNA of 693 nucleotides (FIG. 6 A). The amino acid sequence encoded by the cDNA is 43% identical and 61% similar to a 144 amino acid Cyprinus carpio (common carp) gonadotropin beta chain precursor (SwissProt acc: P01235) (see FIGS. 6B and 7B). PSORT analysis confirmed this sequence is cytoplasmic protein (certainty=0.7953), possibly also localized to lysozyme lumen (certainty=0.4242). The NOVGON nucleic acid sequence of the invention is shown in FIG. 6A. The disclosed sequence is 693 nucleotides in length (SEQ ID NO: 5), and encodes a NOVGON protein of 130 amino acids (SEQ ID NO: 6; also shown in FIG. 6B). Hydrophobicity analysis of the amino acid sequence indicates that NOVGON is a largely hydrophilic, having a distinct hydrophobic domain at its N-terminus, a somewhat hydrophobic domain near its C-terminus. See FIG. 20.

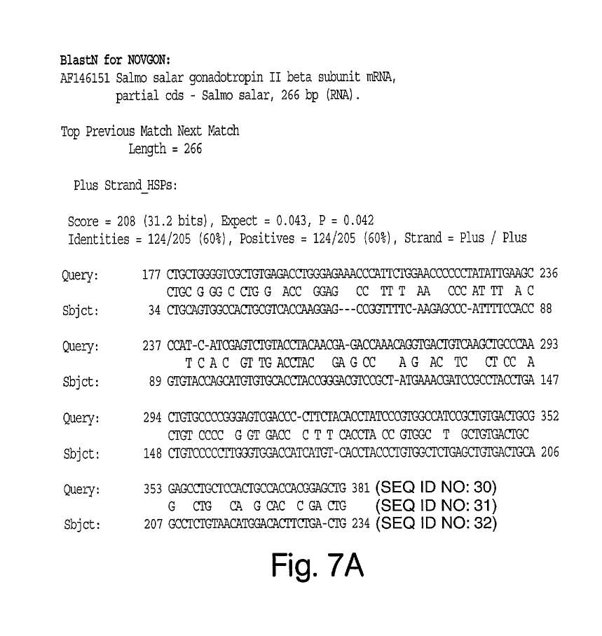

The NOVGON protein disclosed has homology to a number of species variants of the gonadotropin family, including goldfish gonadotropin, and bovine and sheep lutropin (see FIG. 8). NOVGON is 61% similar to carp gonadotropin beta chain precursor at the amino acid level, over residues 42 to 126 of of SEQ ID NO: 6. The NOVGON polypeptide of the invention is comparably as similar (61%) to carp gonadotropin beta chain precursor at the amino acid level as the nearest family member, R. Norvegicus (rat) LSH beta-chain precursor, is to human choriogonadotropin beta-chain (65% identical over 164 a.a.) (see UniGene database, www.ncbi.nlm.nih.gov/UniGene). The protein also is significantly similar to human gonadotropin/bLH chimera, D10 (patp: R15106) (57% at the amino acid level) and Equine chorionic gonadotropin beta-chain protein (patp: R65110) (51% at the amino acid level). See FIG. 7B.

A NOVGON polypeptide of the invention encompasses a mature protein described herein, or mature forms arising therefrom as a result of post-translational modifications. Thus, the proteins of the invention encompass both a precursor and any active forms of the NOVGON protein.

As described in more detail in Example 1, below, the NOVGON nucleic acid sequence of the invention is highly expressed in certain normal tissues and in a melanoma cell line. This suggests that NOVGON may serve as a diagnostic probe for certain specific cancer types, e.g. melanoma, among other uses, as described herein.

### NOVINTRA A, B, & C

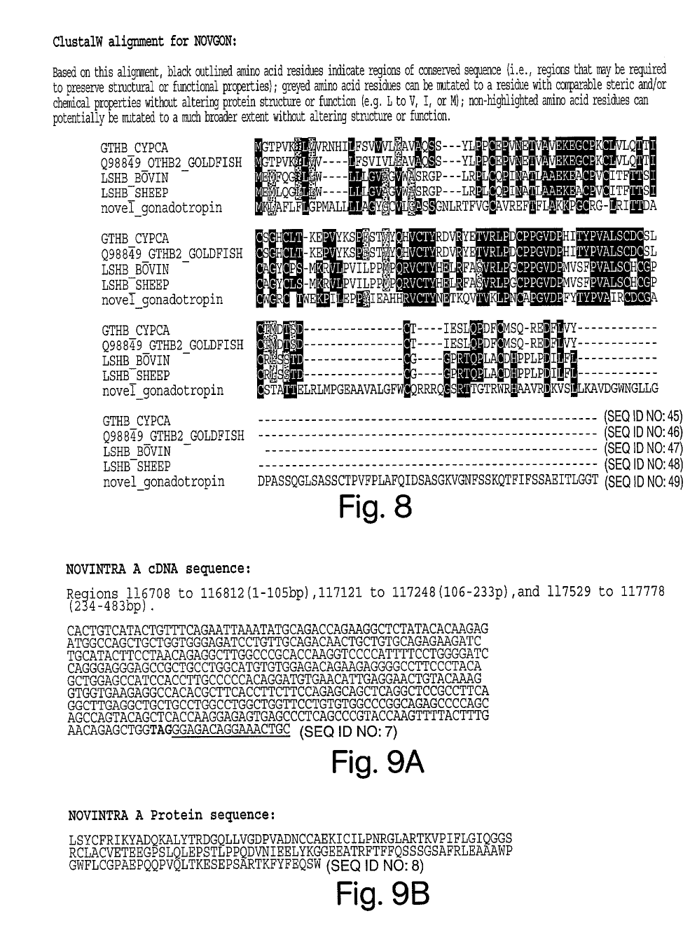

Three novel human interleukin-1 receptor antagonist-like (NOVINTRA) genes were identified based on their homology to Equus callabrus antagonist secretory form (IL-1ra) gene (acc: AF072476) or Sus scrofa IRAP1 mRNA (acc: L38849), respectively (see FIGS. 10A, 13A, and 16A, respectively). Subsequent sequence analysis confirmed that one of these sequences, NOVINTRA C, contains a coding sequence that is identical to a recently-described human interleukin-1 (IL-1) epsilon gene, as described below.

### NOVINTRA A

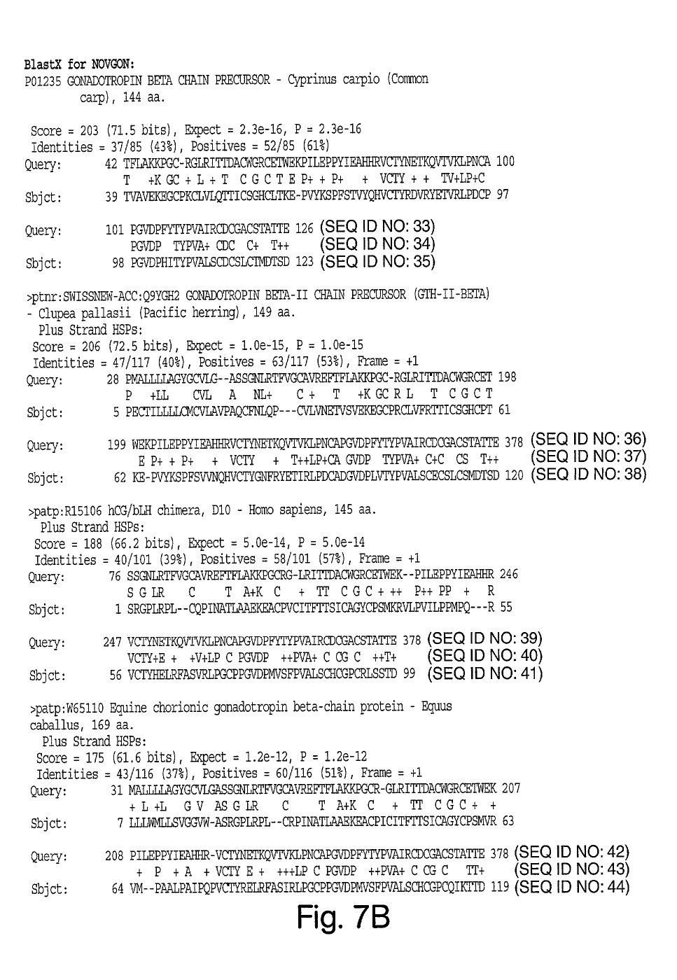

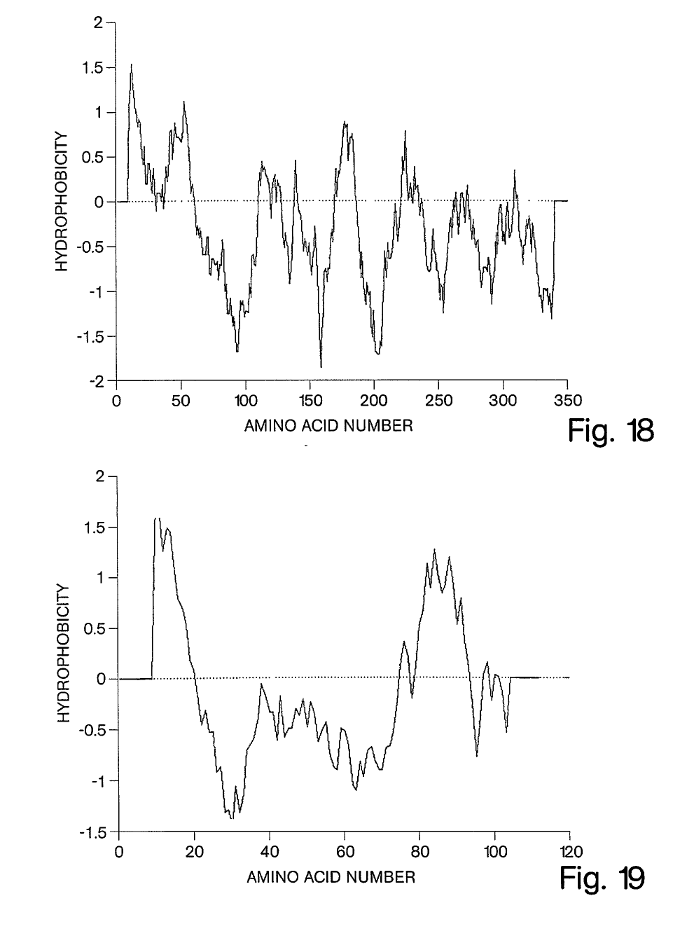

Sequence analysis of the genomic DNA fragment AC016724_A generated a cDNA of 483 nucleotides (FIG. 9A) containing a stop codon (shown in bold in FIG. 9A) at position 465. The amino acid sequence encoded by the cDNA is 46% identical and 62% similar to a 155 amino acid Mus musculus (mouse) IL-IL1 protein (Tremblnew acc: PCAB59831) (see FIGS. 9B and 10B). PSORT analysis confirmed this sequence is a cytoplasmic protein (certainty=0.4500). The NOVINTRA A nucleic acid sequence of the invention is shown in FIG. 9A. The disclosed sequence is 483 nucleotides in length (SEQ ID NO: 7), and contains a 16 nucelotide putative UTR (residues 458 to 473 of SEQ ID NO: 7). The coding sequence encodes a NOVINTRA A protein of 154 amino acids (SEQ ID NO: 8; also shown in FIG. 9B). Hydrophobicity analysis of the amino acid sequence indicates that NOVINTRA A is a largely hydrophilic protein, having several distinct hydrophobic domains clustered near its N-terminus, and a distinctly hydrophilic domain at its C-terminus. See FIG. 21.

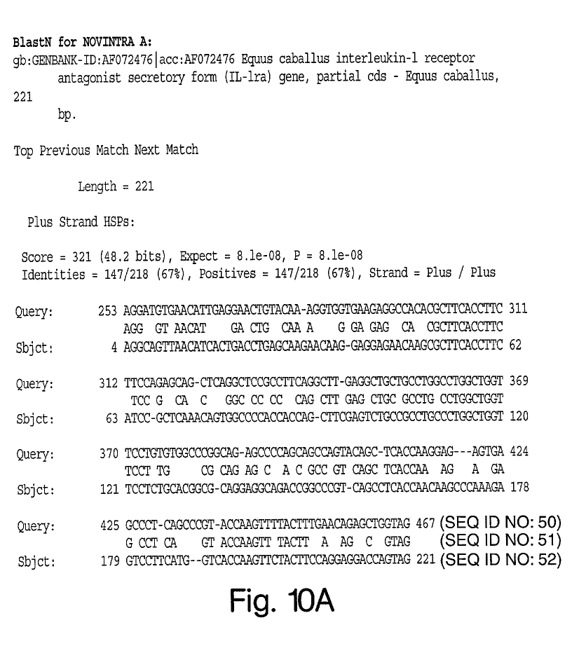

The NOVINTRA A protein disclosed has substantial homology to both human IL-1 delta encoding DNA and intracellular IL-1 receptor antagonist type II, as well as ovine IL-1 beta (see FIG. 11). NOVINTRA A is 62% similar to mouse IL-IL1 protein at the amino acid level, over residues 4 to 152 of of SEQ ID NO: 8. The NOVINTRA A polypeptide of the invention is comparably as similar (62%) to human IL1RN at the amino acid level as the nearest family member, M. musculus (mouse) intracellular IL1 RN, is to human IL1 RN (75% identical over 157 a.a.) (see UniGene database, www.ncbi.nlm.nih.gov/UniGene). The protein also has substantial similarity to human delta interleukin-1 like protein 1 (SPTREMBL-ACC:Q9UBH0)(59% at the amino acid level). Smith et al., J. Biol. Chem. 275: 1169-1175 (2000). See FIG. 10B

### NOVINTRA B

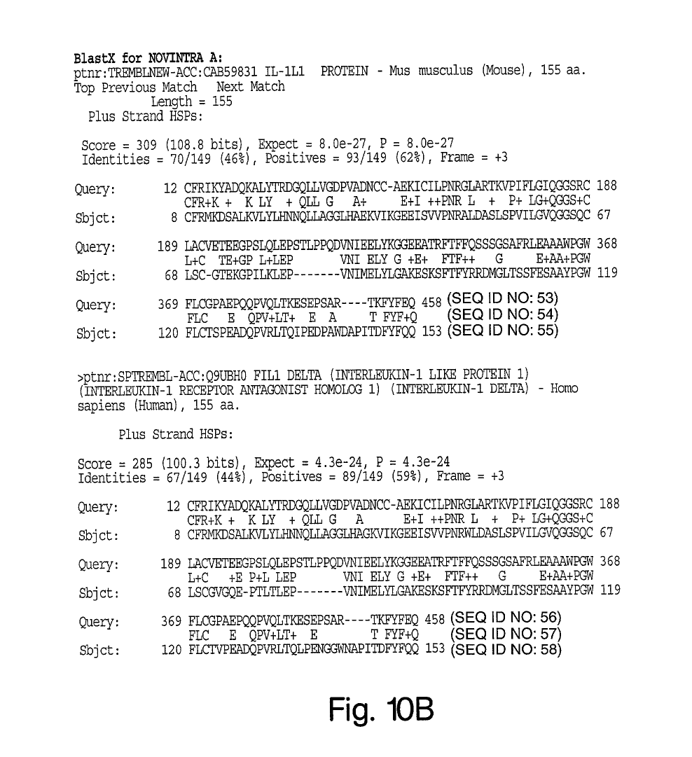

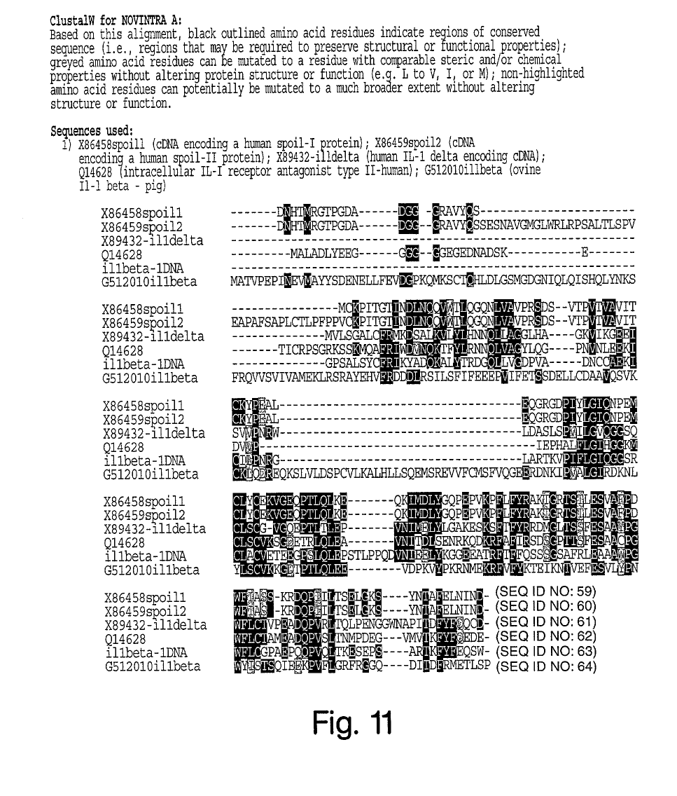

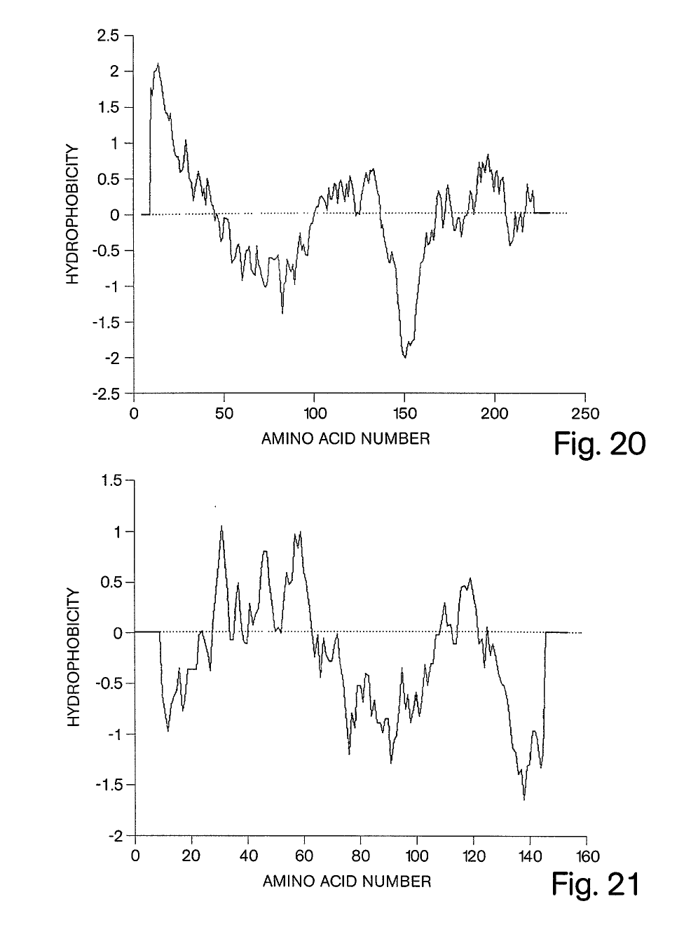

Sequence analysis of the genomic DNA fragment AC016724_B generated a cDNA of 520 nucleotides (FIG. 12A) containing a 513 amino acid coding sequence (stop/start codons shown in bold in FIG. 12A). The amino acid sequence encoded by the cDNA is 100% identical to a 157 amino acid FIL-1 ETA protein (Sptrembl-acc: Q9UHA5; see Smith et al., supra.), and 94% identical and 95% positive to a 164 amino acid human interleukin-1 homolog 2 protein (Sptrembl-acc: Q9NZH7; Kumar et al., J. Biol. Chem. 275: 10308-314 (2000)) (see FIGS. 12A and 13B). NOVINTRA A is also 35% identical and 51% similar to a 155 amino acid human IL1 RN homolog (Tremblnew acc: AAF02757) (see FIG. 13B). PSORT analysis confirmed this sequence is a microbody (peroxisome) protein (certainty=0.5035), possibly also localized to the cytoplasm (certainty=0.4500). The NOVINTRA B nucleic acid sequence of the invention is shown in FIG. 12A. The disclosed sequence is 520 nucleotides in length (SEQ ID NO: 9), and contains a 7 nucelotide putative UTR (residues 514 to 520 of SEQ ID NO: 9). The coding sequence encodes a NOVINTRA B protein of 170 amino acids (SEQ ID NO: 10; also shown in FIG. 12B). Hydrophobicity analysis of the amino acid sequence indicates that NOVINTRA B contains two distinct hydrophobic domains, one near its N-terminus, and the other in the middle of the protein, and has a largely hydrophilic C-terminus. See FIG. 22.

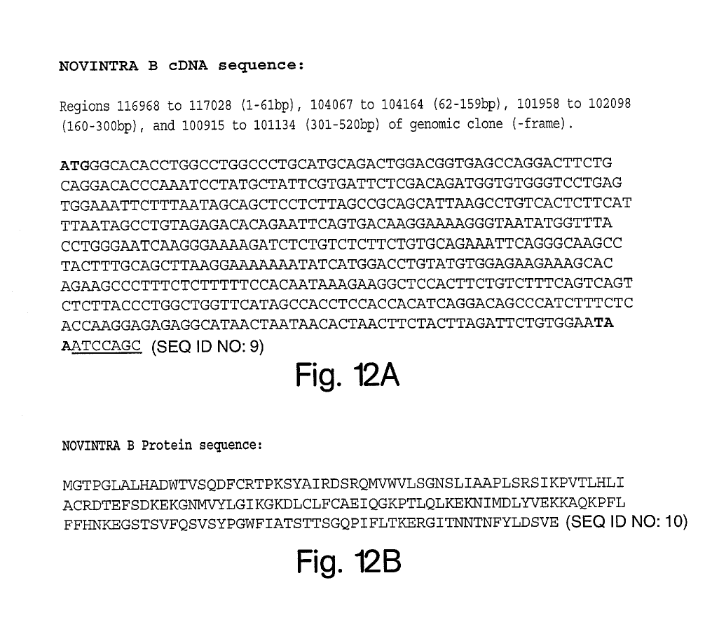

The NOVINTRA B protein disclosed has substantial homology to both human intracellular IL-1 receptor antagonist tyte II and ovine IL-1 beta (see FIG. 14). NOVINTRA B is 51% similar to human IL1 RN homolog at the amino acid level, over residues 25 to 170 of of one of SEQ ID NOs: 10, 100% identical to human FIL-1, a member of the IL-1 superfamily, over residues 21 to 170 of SEQ ID NO: 10, and 94% identical to human IL-1 homolog 2 over residues 21 to 106 of SEQ ID NO: 10. The NOVINTRA B polypeptide of the invention is comparably as similar (51%) to human IL1RN at the amino acid level as the nearest family member, M. musculus (mouse) intracellular IL1 RN, is to human IL1 RN (75% identical over 157 a.a.) (see UniGene database, www.ncbi.nlm.nih.gov/UniGene).

### NOVINTRA C (Novel Human IL-1 epsilon-like protein)

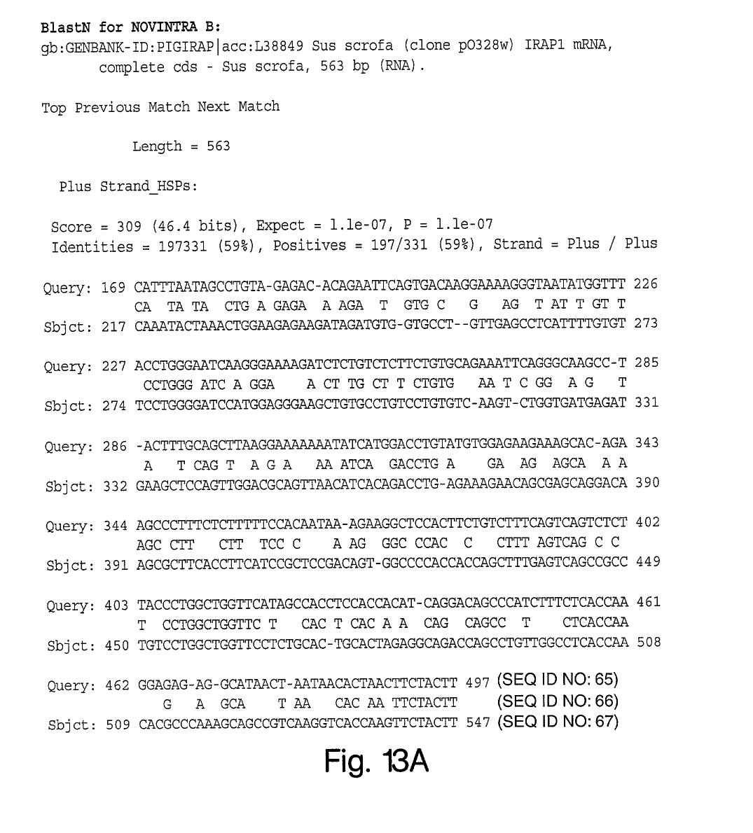

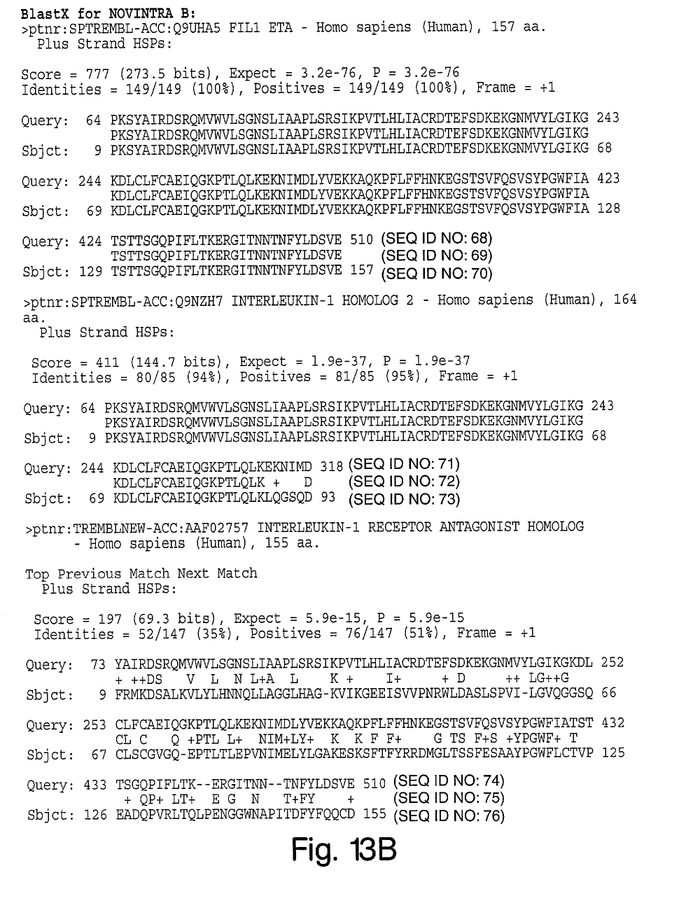

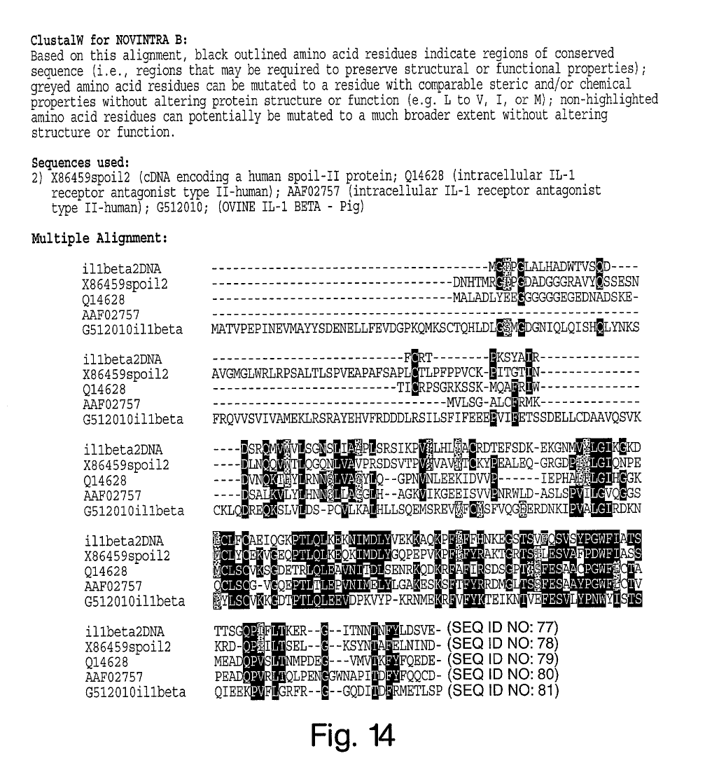

Sequence analysis of the genomic DNA fragment AC016724_C generated a cDNA of 391 nucleotides (FIG. 15A; SEQ ID NO: 11). The amino acid sequence encoded by the cDNA is 96% identical and 97% similar to a 158 amino acid human FIL-1 epsilon protein (Sptrembl-acc: Q9UHA7; see Smith et al., supra.) and 63% identical and 77% positive to a 169 amino acid human IL-1 homolog 1 protein (Sptrebml-acc: Q9NZH8; see Kumar et al., supra.) (See FIGS. 15B and 16B). NOVINTRA C is also 43% identical and 61% similar to a 178 amino acid Mus musculus (mouse) IL-1 receptor antagonist protein precursor (IL-1RA) (SwissProt acc: P25085) (see FIG. 16B).

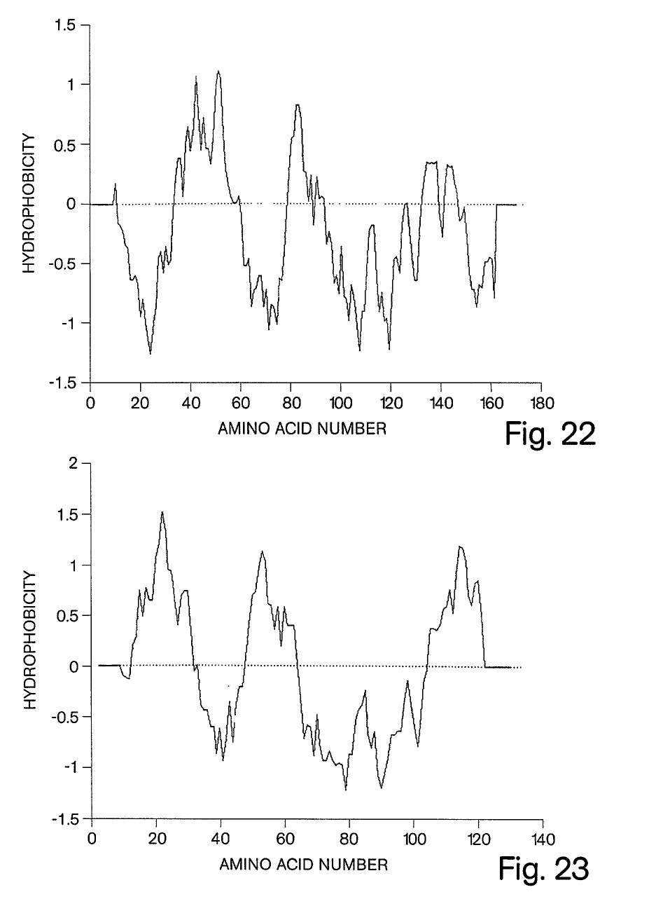

The NOVINTRA C nucleic acid sequence disclosed herein is shown in FIG. 15A, and encodes a NOVINTRA C protein of 130 amino acids (SEQ ID NOs: 12; also shown in FIG. 15B). Hydrophobicity analysis of the amino acid sequence indicates that NOVTINTRA C has three distinct hydrophobic domains, including its N-terminus and C-terminus. See FIG. 23.

As discussed in more detail in Example 1, below, it has been discovered that expression of human IL-1 epsilon-like (also referred to NOVINTRA C protein herein) is highly elevated in lung cancer tissue, and is differentially expressed in TNF-alpha treated small airway epithelium. These results indicate that reagents specific for NOVINTRA C nucleic acids and/or polypeptides. are useful as diagnostic tools for identifying lung cancers. For example, lung cancers can be diagnosed using nucleic acids that detect NOVINTRAC RNA levels in a biolgoical sample, or antibodies (such as monocloclonal antibodies) against NOVINTRA C prrotein. These results also suggest therapeutic interventions for lung cancer by inhibiting expression of a NOVINTRA C gene, or inhibiting the activity of a NOVINTRA C polypeptide.

These results further suggest a role for IL-1 epsilon in asthma, e.g., in mediating irritation in the lungs due to allergies and inflammatory conditions in diseases such as emphysema, and of identifying or treating these conditions using reagents that detect and/or antagonized NOVINTRA expression and or function. Providing an accurate indicator of the presence and measurement of the amount of IL-1 Epsilon may assist in the diagnosis and treatment of asthmatic and allergy patients.

In addition, the expression profile of NOVINTRA C (IL-1 Epsilon) has demonstrated that it has disease association with asthma, allergy and emphysema. It may play a potential role in the development of these diseases. Therefore it has potential usefulness as a therapeutic target, for example, as a target for an IL-1 Epsilon-specific monoclonal antibody, other protein therapeutic or small molecule therapeutic.

A NOVINTRA polypeptide, e.g. NOVINTRA A or B, of the invention encompasses a protein described herein, or mature forms arising therefrom as a result of post-translational modifications. Thus, the proteins of the invention encompass both a precursor and any active forms of the NOVINTRA proteins.

Identification of the new NOVX sequences described herein suggests a variety of uses for the described nucleic acids and polypeptides. For example, transmembrane proteins, such as NOVTRAN, are integral to cell signaling pathways implicated in diseases such as cancer, immune and hematopoietic disorders, and neurodegenerative disease. Neuromedins, such as NOVNEUR, are involved in endocrine, muscle, and neurologic diseases, as well as cancer. Gonadotropins, such as NOVGON, are involved in reproductive development, weight gain/loss, metabolic function, and other hormonally-modulated diseases, such as cancer (e.g. Karposi's sarcoma). Interleukin receptor antagonists, such as NOVINTRA, B and C (IL-1 epsilon), are implicated in disorders of bone metabolism and structure, inflammatory response, and immune regulation, and diseases such as septic shock, stroke, diabetes, arthritis and cancer. Indeed, the differential expression of these genes in certain cancerous tissues, as well as lung tissues, provide novel methods of diagnosing and treating specific disorders, as detailed herein. Accordingly, NOVX nucleic acids and proteins may be useful in the prevention and/or treatment of these diseases, as well as the identification of modulators of these diseases.

Various further utilities for the NOVX nucleic acids and polypeptides are disclosed herein. For example, one use for the NOVX nucleic acids and polypeptides is in methods of identifying compounds that have NOVX-like activities. Such compounds can be identified by contacting a cell containing a NOVX nucleic acids, e.g. NOVINTRA A, and then comparing levels of a NOVX nucleic acid or protein, e.g. NOVINTRA A, to levels of the nucleic acid or protein produced by the cell in the absence of a protein. Another use for the nucleic acids and polypeptides is to identify a subject's responsiveness to a therapeutic agent by examining expression of a NOVX nucleic acid or polypeptide following exposure of a test agent to subject's cells. Higher levels of the NOVX nucleic acid or polypeptide in the presence of the agent compared to the absence of the agent indicates the subject will be responsive to the test agents.

### NOVX Nucleic Acids

The novel nucleic acids provided by the invention include those that encode a NOVX protein, or biologically-active portions thereof. The encoded polypeptides can thus include, e.g., the amino acid sequence of one of SEQ ID NOs: 2, 4, 6, 8, 10, or 12. The novel nucleic acid sequences encoding the NOVX proteins of the invention, e.g. NOVTRAN, NOVNEUR, NOVGON, and NOVINTRA A and B, include the nucleic acid sequences of SEQ ID NOs: 1, 3, 5, 7, 9, and 11, respectively.

In some embodiments, a NOVX nucleic acid according to the invention encodes a mature form of a NOVX protein. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

In some embodiments, a nucleic acid encoding a polypeptide having the amino acid sequence of a NOVX polypeptide includes a nucleic acid sequence selected from SEQ ID NOs: 1, 3, 5, 7 or 9, or a fragment, thereof. Additionally, the invention includes mutant or variant nucleic acids of these sequences, or a fragment thereof, any of whose bases may be changed from the disclosed sequence while still encoding a protein that maintains its NOVX-like biological activities and physiological functions (e.g. gonadotropin-like activity for NOVGON protein). The invention further includes the complement of the nucleic acid sequence of a NOVX nucleic acid, e.g., SEQ ID NOs: NOs: 1, 3, 5, 7 or 9, including fragments, derivatives, analogs and homologs thereof. The invention additionally includes nucleic acids or nucleic acid fragments, or complements thereto, whose structures include chemical modifications.

Also included are nucleic acid fragments sufficient for use as hybridization probes to identify NOVX protein-encoding nucleic acids (e.g., NOVX mRNA) and fragments for use as polymerase chain reaction (PCR) primers for the amplification or mutation of NOVX protein nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments, and homologs thereof. The nucleic acid molecule can be single-stranded or double-stranded, but preferably is double-stranded DNA.

The term “probes” refer to nucleic acid sequences of variable length, preferably between at least about 10 nucleotides (nt), 100 nt, or as many as about, e.g., 6,000 nt, depending upon the specific use. Probes are used in the detection of identical, similar, or complementary nucleic acid sequences. Longer length probes are usually obtained from a natural or recombinant source, are highly specific and much slower to hybridize than oligomers. Probes may be single- or double-stranded, and may also be designed to have specificity in PCR, membrane-based hybridization technologies, or ELISA-like technologies.

The term “isolated” nucleic acid molecule is a nucleic acid that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecule can contain less than approximately 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of one of SEQ ID NOs: 1, 3, 5, 7, 9, or 11, or a complement of this nucleotide sequence, can be isolated using standard molecular biology techniques and the sequence information provided herein. Using all or a portion of the nucleic acid sequence of one of SEQ ID NOs: 1, 3, 5, 7, 9, or 11 as a hybridization probe, NOVX protein-encoding nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of one of SEQ ID NOs: 1, 3, 5, 7, 9, or 11, or a complement thereof. Oligonucleotides may be chemically synthesized and may also be used as probes.

In another embodiment, an isolated nucleic acid molecule of the invention includes a nucleic acid molecule that is a complement of the nucleotide sequence shown in any of one of SEQ ID NOs: 1, 3, 5, 7, 9, or 11. In still another embodiment, an isolated nucleic acid molecule of the invention includes a nucleic acid molecule that is a complement of the nucleotide sequence shown in any of one of SEQ ID NOs: 1, 3, 5, 7, 9, or 11, or a portion of this nucleotide sequence. A nucleic acid molecule that is complementary to the nucleotide sequence shown in one of SEQ ID NOs: 1, 3, 5, 7, 9, or 11 is one that is sufficiently complementary to the nucleotide sequence shown in one of SEQ ID NOs: 1, 3, 5, 7, 9, or 11 that it can hydrogen bond with little or no mismatches to the nucleotide sequence shown in one of SEQ ID NOs: 1, 3, 5, 7, 9, or 11, thereby forming a stable duplex.

As used herein, the term “complementary” refers to Watson-Crick or Hoogsteen base-pairing between nucleotides units of a nucleic acid molecule, whereas the term “binding” is defined as the physical or chemical interaction between two polypeptides or compounds or associated polypeptides or compounds or combinations thereof. Binding includes ionic, non-ionic, Von der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Additionally, the nucleic acid molecule of the invention can comprise only a portion of the nucleic acid sequence of any of one of SEQ ID NOs: 1, 3, 5, 7, 9, or 11, e.g., a fragment that can be used as a probe or primer, or a fragment encoding a biologically active portion of a NOVX protein. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild-type.

Derivatives and analogs may be full-length or other than full-length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489), which is incorporated herein by reference in its entirety.

The term “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, e.g., alternative splicing of RNA. Alternatively, isoforms can be encoded by different genes. In the invention, homologous nucleotide sequences include nucleotide sequences encoding for a NOVX polypeptide of species other than humans, including, but not limited to, mammals, and thus can include, e.g., mouse, rat, rabbit, dog, cat cow, horse, and other organisms. Homologous nucleotide sequences also include, but are not limited to, naturally-occurring allelic variations and mutations of the nucleotide sequences set forth herein. A homologous nucleotide sequence does not, however, include the nucleotide sequence encoding a NOVX protein. Homologous nucleic acid sequences include those nucleic acid sequences that encode conservative amino acid substitutions (see below) in one of SEQ ID NOs: 1, 3, 5, 7 or 9, as well as a polypeptide having NOVX-like activity, e.g. hormonal activity of NOVGON, as described above. A homologous amino acid sequence does not encode the amino acid sequence of a NOVX protein.

The nucleotide sequence disclosed for the NOVX protein gene allows for the generation of probes and primers designed for use in identifying NOVX protein-expressing cell types, e.g. liver cells, and/or cloning NOVX protein homologues in other cell types, e.g., from other tissues, as well as NOVX protein homologues from other mammals. The probe/primer typically includes a substantially-purified oligonucleotide. The oligonucleotide typically includes a region of nucleotide sequence that hybridizes under stringent conditions to at least about 12, 25, 50, 100, 150, 200, 250, 300, 350 or 400 or more consecutive sense strand nucleotide sequence of a NOVX nucleic acid, e.g., one including all or a portion of one of SEQ ID NOs: 1, 3, 5, 7, 9, or 11. Alternatively, the oligonucleotide sequence may include a region of nucleotide sequences that hybridizes to some or all of an anti-sense strand of a strand encoding NOVX nucleic acid. For example, the oligonucleotide may include some or all of the anti-sense strand nucleotide sequence of one of SEQ ID NOs: 1, 3, 5, 7, 9, or 11, or of a naturally occurring mutant of one of these nucleic acids.

Probes based upon the NOVX nucleotide sequence can be used to detect transcripts or genomic sequences encoding the same or homologous proteins. In various embodiments, the probe further includes a label group attached thereto, e.g., the label group can be a radioisotope, a fluorescent compound, an enzyme, or an enzyme co-factor. Such probes can be used as a part of a diagnostic test kit for identifying cells or tissue (e.g. liver) which mis-express a NOVX protein, such as by measuring a level of a NOVX protein-encoding nucleic acid in a sample of cells from a subject e.g., detecting NOVX mRNA levels or determining whether a genomic NOVX gene has been mutated or deleted.

The term “a polypeptide having a biologically-active portion of NOVX protein” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX protein” can be prepared by isolating a portion of a nucleotide, e.g., a nucleotide including a portion of one of SEQ ID NOs: 1, 3, 5, 7, 9, or 11, that encodes a polypeptide having NOVX-like biological activity (as described above), expressing the encoded portion of a NOVX protein (e.g., by recombinant expression in vitro) and assessing the activity of the encoded portion of a NOVX protein.

### NOVX Nucleic Acid Variants

The invention further encompasses nucleic acid molecules that differ from the disclosed NOVX nucleotide sequence due to degeneracy of the genetic code. These nucleic acids can encode the same NOVX protein as those encoded by the nucleotide sequence of one of SEQ ID NOs: 1, 3, 5, 7, 9, or 11. In another embodiment, an isolated nucleic acid molecule of the invention has a nucleotide sequence encoding a protein having the amino acid sequence of one of SEQ ID NOs: 2, 4, 6, 8, 10, or 12.

In addition to the NOVX nucleotide sequences shown in SEQ ID NOs: 1, 3, 5, 7, 9, or 11 it will be appreciated by those skilled in the art that DNA sequence polymorphisms that lead to changes in the amino acid sequence of a NOVX protein may exist within a population (e.g., the human population). Such genetic polymorphism in the NOVX protein gene may exist among individuals within a population due to natural allelic variation. As used herein, the terms “gene” and “recombinant gene” refer to nucleic acid molecules comprising an open reading frame encoding a NOVX protein, preferably a mammalian protein. Such natural allelic variations can typically result in 1-5% variance in the nucleotide sequence of the NOVX gene. Any and all such nucleotide variations and resulting amino acid polymorphisms in NOVX protein that are the result of natural allelic variation and that do not alter the functional activity of NOVX protein are intended to be within the scope of the invention.

Additionally, nucleic acid molecules encoding NOVX protein proteins from other species, and thus that have a nucleotide sequence that differs from the nucleic acid sequence of a NOVX protein (e.g., it differs from one of SEQ ID NOs: 1, 3, 5, 7, 9, or 11), are intended to be within the scope of the invention. Nucleic acid molecules corresponding to natural allelic variants and homologues of the NOVX cDNAs of the invention can be isolated based on their homology to the NOVX protein-encoding nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of a NOVX nucleic acid, e.g., one of SEQ ID NOs: 1, 3, 5, 7, 9, or 11. In another embodiment, the nucleic acid is at least 10, 25, 50, 100, 250, 500 or 750 nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of a NOVX nucleic acid, including those described herein, corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of a NOVX nucleic acid (e.g., one of SEQ ID NOs: 1, 3, 5, 7, 9, or 11), or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990. GENE TRANSFER AND EXPRESSION, A LABORATORY MANUAL, Stockton Press, N.Y.

In a third embodiment, a nucleic acid that is hybridizable to the nucleic acid molecule comprising the nucleotide sequence of a NOVX nucleic acid (e.g., it hybridizes to one of SEQ ID NOs: 1, 3, 5, 7, 9, or 11), or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al., (eds.), 1993. CURRENT PROTOCOLS IN MOLECULAR BIOLOGY, John Wiley & Sons, NY, and Kriegler, 1990. GENE TRANSFER AND EXPRESSION, A LABORATORY MANUAL, Stockton Press, NY; Shilo and Weinberg, 1981. Proc. Natl. Acad. Sci. USA 78: 6789-6792.

### Conservative Mutations

In addition to naturally-occurring allelic variants of the NOVX protein-encoding sequence that may exist in the population, the skilled artisan will further appreciate that changes can be introduced by mutation into the nucleotide sequence of a NOVX nucleic acid (e.g., one of SEQ ID NOs: 1, 3, 5, 7, 9, or 11), thereby leading to changes in the amino acid sequence of the encoded NOVX protein, without altering the functional ability of the protein. For example, nucleotide substitutions leading to amino acid substitutions at “non-essential” amino acid residues can be made in the sequence of one of SEQ ID NOs: 1, 3, 5, 7, 9, or 11. A “non-essential” amino acid residue is a residue that can be altered from the wild-type sequence of NOVX protein without altering the biological activity, whereas an “essential” amino acid residue is required for biological activity. For example, amino acid residues that are conserved among respective NOVX protein families, including the NOVX proteins of the invention, are predicted to be particularly non-amenable to such alteration (see, e.g. FIG. 10).

Amino acid residues that are conserved among members of the respective protein family are predicted to be less amenable to alteration. For example, a NOVX protein according to the invention can contain at least one consensus domain that is a typically conserved region of a NOVX protein family member. See e.g. FIG. 10. As such, these conserved domains are not likely to be amenable to mutation. Other amino acid residues, however, (e.g., those that are not conserved or only semi-conserved among members of the NOVX protein family) may not be as essential for activity and thus are more likely to be amenable to alteration.

Another aspect of the invention pertains to nucleic acid molecules encoding NOVX proteins that contain changes in amino acid residues that are not essential for activity. Such proteins differ in amino acid sequence from the amino acid sequence of a NOVX protein (e.g., one of SEQ ID NOs: 2, 4, 6, 8, 10, or 12), yet retain biological activity. In one embodiment, the isolated nucleic acid molecule includes a nucleotide sequence encoding a protein, wherein the protein includes an amino acid sequence at least about 75% homologous to the amino acid sequence of any of one of SEQ ID NOs: 2, 4, 6, 8, 10, or 12. Preferably, the protein encoded by the nucleic acid is at least about 80% homologous to any of one of SEQ ID NOs: 2, 4, 6, 8, 10, or 12, more preferably at least about 90%, 95%, 98%, and most preferably at least about 99% homologous to one of SEQ ID NOs: 2, 4, 6, 8, 10, or 12.

An isolated nucleic acid molecule encoding a NOVX protein homologous to a NOVX protein, e.g. a polypeptide including the amino acid sequence of any of one of SEQ ID NOs: 2, 4, 6, 8, 10, or 12, can be created by introducing one or more nucleotide substitutions, additions or deletions into the corresponding NOVX nucleotide sequence, such that one or more amino acid substitutions, additions or deletions are introduced into the encoded protein.

Mutations can be introduced into NOVX protein-encoding nucleic acid by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Thus, a predicted nonessential amino acid residue in a NOVX protein is replaced with another amino acid residue from the same side chain family. Alternatively, in another embodiment, mutations can be introduced randomly along all or part of a NOVX protein coding sequence, such as by saturation mutagenesis, and the resultant mutants can be screened for NOVX protein biological activity to identify mutants that retain activity. Following mutagenesis of the NOVX nucleic acid, the encoded protein can be expressed by any recombinant technology known in the art and the activity of the protein can be determined.

In one embodiment, a mutant NOVX protein can be assayed for: (i) the ability to form protein:protein interactions with other NOVX proteins, other cell-surface proteins, or biologically-active portions thereof; (ii) complex formation between a mutant NOVX protein and a NOVX protein receptor; (iii) the ability of a mutant NOVX protein to bind to an intracellular target protein or biologically active portion thereof; (e.g., avidin proteins); (iv) the ability to bind BRA protein; or (v) the ability to specifically bind an anti-NOVX protein antibody.

### Antisense Nucleic Acids

Another aspect of the invention pertains to isolated antisense nucleic acid molecules that are hybridizable to or complementary to the nucleic acid molecule including a NOVX nucleic acid (e.g. a nucleic acid including one of SEQ ID NOs: 1, 3, 5, 7, 9, or 11), or fragments, analogs or derivatives thereof. An “antisense” nucleic acid includes a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire NOVX protein coding strand, or to only a portion thereof.

In one embodiment, an antisense nucleic acid molecule is antisense to a “coding region” of the coding strand of a nucleotide sequence encoding NOVX protein. The term “coding region” refers to the region of the nucleotide sequence comprising codons which are translated into amino acid residues (e.g., one of SEQ ID NOs: 1, 3, 5, 7, 9, or 11). In another embodiment, the antisense nucleic acid molecule is antisense to a “non-coding region” of the coding strand of a NOVX nucleotide sequence. The term “non-coding region” refers to 5′ and 3′ sequences which flank the coding region that are not translated into amino acids (i.e., also referred to as 5′ and 3′ non-translated regions).

Given the coding strand sequences encoding NOVX proteins disclosed herein, antisense nucleic acids of the invention can be designed according to the rules of Watson and Crick or Hoogsteen base-pairing. The antisense nucleic acid molecule can be complementary to the entire coding region of a NOVX protein mRNA, but more preferably is an oligonucleotide that is antisense to only a portion of the coding or non-coding region of NOVX protein mRNA. For example, the antisense oligonucleotide can be complementary to the region surrounding the translation start site of a NOVX protein mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine-substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other (Gaultier, et al., 1987. Nucl. Acids Res. 15: 6625-6641). The antisense nucleic acid molecule can also comprise a 2′-o-methylribonucleotide (Inoue, et al., 1987. Nucl. Acids Res. 15: 6131-6148) or a chimeric RNA-DNA analogue (Inoue, et al., 1987. FEBS Lett. 215: 327-330).

### Ribozymes and PNA Moieties

Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes; described by Haselhoff and Gerlach, 1988. Nature 334: 585-591) can be used to catalytically-cleave NOVX protein mRNA transcripts to thereby inhibit translation of NOVX protein mRNA. A ribozyme having specificity for a NOVX nucleic acid can be designed based upon the nucleotide sequence of NOVX protein DNA disclosed herein (e.g., one of SEQ ID NOs: 1, 3, 5, 7, 9, or 11). For example, a derivative of a Tetrahymena L-19 IVS RNA can be constructed in which the nucleotide sequence of the active site is complementary to the nucleotide sequence to be cleaved in a NOVX protein-encoding mRNA. See, e.g., Cech, et al., U.S. Pat. No. 4,987,071; and Cech, et al., U.S. Pat. No. 5,116,742. Alternatively, NOVX protein mRNA can be used to select a catalytic RNA having a specific ribonuclease activity from a pool of RNA molecules (Bartel, et al., 1993. Science 261: 1411-1418).

Alternatively, NOVX protein gene expression can be inhibited by targeting nucleotide sequences complementary to the regulatory region of the NOVX nucleic acid (e.g., the promoter and/or enhancers) to form triple helical structures that prevent transcription of the NOVX protein gene in target cells. See, e.g., Helene, 1991. Anticancer Drug Des. 6: 569-84; Helene, et al., 1992. Ann. N.Y Acad. Sci. 660: 27-36; and Maher, 1992. Bioassays 14: 807-15.

In various embodiments, the nucleic acids of NOVX protein can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (Hyrup, et al., 1996. Bioorg. Med. Chem. 4: 5-23). As used herein, the terms “peptide nucleic acids” or “PNAS” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. above; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, e.g., in the analysis of single base pair mutations in a gene by, e.g., PNA directed PCR clamping; as artificial restriction enzymes when used in combination with other enzymes, e.g., SI nucleases (see, Hyrup, 1996., above); or as probes or primers for DNA sequence and hybridization (see, Hyrup, et al., 1996.; Perry-O'Keefe, 1996., above).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, 1996., above). The synthesis of PNA-DNA chimeras can be performed as described in Finn, et al., (1996. Nucl. Acids Res. 24: 3357-3363). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag, et al., 1989. Nucl. Acid Res. 17: 5973-5988). PNA monomers are then coupled in a stepwise manner to produce a chimeric molecule with a 5′ PNA segment and a 3′ DNA segment (see, Finn, et al., 1996., above). Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad Sci. USA. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

### NOVX Polypeptides

A polypeptide according to the invention includes a polypeptide including the amino acid sequence of a NOVX polypeptide. In some embodiments, the NOVX polypeptide includes the amino acid sequence of one of SEQ ID NOs: 2, 4, 6, 8, 10, or 12. In various embodiments, a NOVX polypeptide is provided in a form longer than the sequence of the mature NOVX protein. For example, the polypeptide may be provided as including an amino terminal signal sequence. In other embodiments, the NOVX polypeptide is provided as the mature form of the polypeptide.

The invention also includes a mutant or variant protein any of whose residues may be changed from the corresponding residues shown in one of SEQ ID NOs: 2, 4, 6, 8, 10, or 12, while still encoding a protein that maintains its respective NOVX-like activities, e.g. hormonal activity for NOVGON, and physiological functions, or a functional fragment thereof.

In general, a NOVX protein variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to an isolated NOVX protein, as described above, and biologically-active portions thereof, or derivatives, fragments, analogs or homologs thereof. Also provided are polypeptide fragments suitable for use as immunogens to raise anti-NOVX protein antibodies. In one embodiment, native NOVX protein can be isolated from cells or tissue sources by an appropriate purification scheme using standard protein purification techniques. In another embodiment, a NOVX protein is produced by recombinant DNA techniques. Alternative to recombinant expression, NOVX proteins or polypeptides can be synthesized chemically using standard peptide synthesis techniques.

An “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of a NOVX protein in which the protein is separated from cellular components of the cells from which it is isolated or recombinantly-produced. In one embodiment, the language “substantially free of cellular material” includes preparations of a NOVX protein having less than about 30% (by dry weight) of a non-NOVX protein (also referred to herein as a “contaminating protein”), more preferably less than about 20% of a contaminating protein, still more preferably less than about 10% of a contaminating protein, and most preferably less than about 5% of a contaminating protein. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The phrase “substantially free of chemical precursors or other chemicals” includes preparations of NOVX protein in which the protein is separated from chemical precursors or other chemicals that are involved in the synthesis of the protein. In one embodiment, the language “substantially free of chemical precursors or other chemicals” includes preparations of NOVX protein having less than about 30% (by dry weight) of chemical precursors or non-NOVX chemicals (also referred to herein as “chemical contaminants”), more preferably less than about 20% chemical contaminants, still more preferably less than about 10% chemical contaminants, and most preferably less than about 5% chemical contaminants.

Biologically-active portions of a protein include peptides comprising amino acid sequences sufficiently homologous to or derived from the amino acid sequence of the NOVX protein which include fewer amino acids than the full-length protein, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acids in length.

A biologically-active portion of the NOVX protein of the invention may contain at least one domain, e.g. a consensus sequence, conserved among members of the protein family. Moreover, other biologically active portions, in which other regions of the protein are deleted, can be prepared by recombinant techniques and evaluated for one or more of the functional activities of a native NOVX protein.

In some embodiments, the NOVX protein has a sequence which is substantially homologous to one of SEQ ID NOs: 2, 4, 6, 8, 10, or 12, and retains the functional activity of the protein, yet differs in amino acid sequence due to natural allelic variation or mutagenesis, as described in detail below. Accordingly, in another embodiment, the NOVX protein is a protein that includes an amino acid sequence at least about 45% homologous, and more preferably about 55, 65, 70, 75, 80, 85, 90, 95, 98 or even 99% homologous to the amino acid sequence of one of SEQ ID NOs: 2, 4, 6, 8, 10, or 12, and retains the functional activity of the corresponding NOVX protein having the sequence of one of SEQ ID NOs: 2, 4, 6, 8, 10, or 12.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol. Biol. 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOs: 1, 3, 5, 7, 9, 11, 13, 15, 17, 19, 21,or23.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide includes a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides NOVX protein chimeric or fusion proteins. As used herein, a NOVX “chimeric protein” or “fusion protein” includes a NOVX polypeptide operatively-linked to a non-NOVX polypeptide. An “NOVX protein or polypeptide” refers to a polypeptide having an amino acid sequence corresponding to a NOVX protein shown in, e.g., one of SEQ ID NOs: 2, 4, 6, 8, 10, or 12. A “non-NOVX polypeptide” or “non-NOVX protein” refers to a polypeptide having an amino acid sequence corresponding to a protein that is not substantially homologous to a NOVX polypeptide (e.g., a protein that is different from the NOVX protein and that is derived from the same or a different organism). Within a NOVX fusion protein the NOVX polypeptide can correspond to all or a portion of a NOVX protein. In one embodiment, the fusion protein includes at least one biologically-active portion of a NOVX protein . In another embodiment, the fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the amino-terminus or carboxyl-terminus of the NOVX polypeptide.

In one embodiment, the fusion protein is a GST-NOVX fusion protein in which the NOVX sequence is fused to the carboxyl-terminus of the GST (glutathione S-transferase) sequence. Such fusion proteins can facilitate the purification of recombinant NOVX proteins or polypeptides.

In another embodiment, the fusion protein is a NOVX protein containing a heterologous signal sequence at its amino-terminus. In certain host cells (e.g., mammalian host cells), expression and/or secretion of NOVX protein can be increased through use of a heterologous signal sequence.

In yet another embodiment, the fusion protein is a NOVX-immunoglobulin fusion protein in which the NOVX sequence is fused to a sequence derived from a member of the immunoglobulin protein family. The NOVX-immunoglobulin fusion proteins of the invention can be incorporated into pharmaceutical compositions and administered to a subject to inhibit an interaction between a NOVX ligand and, e.g., a NOVX protein on the surface of a cell, to thereby suppress NOVX protein-mediated signal transduction in vivo. The immunoglobulin fusion proteins can be used to affect the bioavailability of a NOVX protein cognate ligand. Inhibition of the ligand/interaction may be useful therapeutically for both the treatment of proliferative and differentiative disorders, as well as modulating (e.g., promoting or inhibiting) cell survival. Moreover, the NOVX-immunoglobulin fusion proteins of the invention can be used as immunogens to produce anti-NOVX protein antibodies in a subject, to purify NOVX ligands, and in screening assays to identify molecules that inhibit the interaction of NOVX protein with a ligand.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX protein-encoding nucleic acid can be cloned into such an expression vector such that the fusion moiety is linked in-frame to the NOVX protein.

### NOVX Protein Agonists and Antagonists

The invention also pertains to variants of a NOVX protein that function as either NOVX protein agonists (i.e., mimetics) or as NOVX protein antagonists. Variants of the NOVX protein can be generated by mutagenesis (e.g., discrete point mutation or truncation of the protein). An agonist of a NOVX protein can retain substantially the same, or a subset of, the biological activities of the naturally-occurring form of a NOVX protein. An antagonist of a NOVX protein can inhibit one or more of the activities of the naturally occurring form of the protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX protein.

Variants of the NOVX protein that function as either agonists (i.e., mimetics) or as antagonists can be identified by screening combinatorial libraries of mutants (e.g., truncation mutants) of the NOVX protein for NOVX protein agonist or antagonist activity. In one embodiment, a variegated library of variants is generated by combinatorial mutagenesis at the nucleic acid level and is encoded by a variegated gene library. A variegated library of NOVX protein variants can be produced by, for example, enzymatically-ligating a mixture of synthetic oligonucleotides into gene sequences such that a degenerate set of potential NOVX protein sequences is expressible as individual polypeptides, or alternatively, as a set of larger fusion proteins (e.g., for phage display) containing the set of NOVX protein sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX protein sequences. Methods for synthesizing degenerate oligonucleotides are well-known within the art. See, e.g. Narang, 1983. Tetrahedron 39: 3; Itakura, et al., 1984. Annu. Rev. Biochem. 53: 323; Itakura, et al., 1984. Science 198: 1056; Ike, et al., 1983. Nuc. Acids Res. 11: 477.

### Polypeptide Libraries

In addition, libraries of fragments of the NOVX protein coding sequence can be used to generate a variegated population of fragments for screening and subsequent selection of variants of a NOVX protein. In one embodiment, a library of coding sequence fragments can be generated by treating a double-stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes amino-terminal and internal fragments of various sizes of the NOVX protein.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX protein. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX protein variants. See, e.g., Arkin and Yourvan, 1992. Proc. Natl. Acad. Sci. USA 89: 7811-7815; Delgrave, et al., 1993. Protein Engineering 6:327-331.

### Anti-NOVX Protein Antibodies

The invention encompasses antibodies and antibody fragments, such as F_{ab} or (F_{ab})₂, that bind immunospecifically to a NOVX protein or polypeptide of the invention.

An isolated NOVX protein, or a portion or fragment thereof, can be used as an immunogen to generate antibodies that bind to NOVX polypeptides using standard techniques for polyclonal and monoclonal antibody preparation. The full-length NOVX protein can be used or, alternatively, the invention provides antigenic peptide fragments of the proteins for use as immunogens. The antigenic peptides comprise at least 4 amino acid residues of a NOVX polypeptide, e.g., the amino acid sequence of one of SEQ ID NOs: 2, 4, 6, 8, 10, or 12, and encompasses an epitope of NOVX protein such that an antibody raised against the peptide forms a specific immune complex with the protein. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of a NOVX protein that is located on the surface of the protein (e.g., a hydrophilic region). As a means for targeting antibody production, hydropathy plots showing regions of hydrophilicity and hydrophobicity may be generated by any method well known in the art, including, for example, the Kyte-Doolittle or the Hopp-Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

NOVX protein sequences including, e.g., one of SEQ ID NOs: 2, 4, 6, 8, 10, or 12, or derivatives, fragments, analogs, or homologs thereof, may be used as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (i.e., immunoreacts with) an antigen, such as NOVX proteins. Such antibodies include, but are not limited to, polyclonal, monoclonal, chimeric, single chain, F_{ab} and F(_{ab′})₂ fragments, and an F_{ab} expression library. In a specific embodiment, antibodies to NOVX protein are disclosed. Various procedures known within the art may be used for the production of polyclonal or monoclonal antibodies to a NOVX protein sequence, e.g., one of SEQ ID NOs: 2, 4, 6, 8, 10, or 12, or a derivative, fragment, analog, or homolog thereof.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX protein can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of a NOVX protein. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to a NOVX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for a NOVX protein or derivatives, fragments, analogs or homologs thereof Non-human antibodies can be “humanized” by techniques well-known within the art. See, e.g., U.S. Pat. No. 5,225,539. Antibody fragments that contain the idiotypes to a NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-NOVX protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314:446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a NOVX protein is facilitated by generation of hybridomas that bind to the fragment of the protein possessing such a domain. Thus, antibodies that are specific for a desired domain within a NOVX protein, or derivatives, fragments, analogs or homologs thereof, are also provided herein.

Anti-NOVX protein antibodies may be used in methods known within the art relating to the localization and/or quantitation of the protein (e.g., for use in measuring levels of the NOVX protein within appropriate physiological samples, for use in diagnostic methods, for use in imaging the protein, and the like). In a given embodiment, antibodies for a protein of the invention, or derivatives, fragments, analogs or homologs thereof, that contain the antibody derived binding domain, are utilized as pharmacologically-active compounds (hereinafter “Therapeutics”).

An anti-NOVX protein antibody (e.g., monoclonal antibody) can be used to isolate an NOVX polypeptide by standard techniques, such as affinity chromatography or immunoprecipitation. An anti-NOVX protein antibody can facilitate the purification of natural NOVX polypeptide from cells and of recombinantly-produced polypeptide expressed in host cells. Moreover, an anti-NOVX protein antibody can be used to detect NOVX protein (e.g., in a cellular lysate or cell supernatant) in order to evaluate the abundance and pattern of expression of the protein. Anti-NOVX protein antibodies can be used diagnostically to monitor protein levels in tissue as part of a clinical testing procedure, e.g., to, for example, determine the efficacy of a given treatment regimen. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### Recombinant Expression Vectors and Host Cells

Another aspect of the invention pertains to vectors, preferably expression vectors, containing a nucleic acid encoding NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably, as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The phrase “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutants, fusion proteins, etc.).

The recombinant expression vectors of the invention can be designed for expression of NOVX protein in prokaryotic or eukaryotic cells. For example, proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T₇ promoter regulatory sequences and T₇ polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xₐ, thrombin, and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion Escherichia coli expression vectors include pTrc (Amrann et al., (1988) Gene 69:301-315) and pET 11d (Studier, et al., GENE EXPRESSION TECHNOLOGY: METHODS IN ENZYMOLOGY 185, Academic Press, San Diego, Calif. (1990) 60-89).

One strategy to maximize recombinant protein expression in Escherichia coli is to express the protein in a host bacteria with an impaired capacity to proteolytically-cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in Escherichia coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX protein expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX proteins can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid), e.g. liver cells. Tissue-specific regulatory elements are known in the art. Non-limiting examples of suitable tissue-specific promoters include the albumin promoter (liver-specific; see, Pinkert, et al., 1987. Genes Dev. 1: 268-277), lymphoid-specific promoters (see, Calame and Eaton, 1988. Adv. Immunol. 43: 235-275), in particular promoters of T cell receptors (see, Winoto and Baltimore, 1989. EMBO J. 8: 729-733) and immunoglobulins (see, Banerji, et al., 1983. Cell 33: 729-740; Queen and Baltimore, 1983. Cell 33: 741-748), neuron-specific promoters (e.g., the neurofilament promoter; see, Byrne and Ruddle, 1989. Proc. Natl. Acad. Sci. USA 86: 5473-5477), pancreas-specific promoters (see, Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (see, Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX proteins can be expressed in bacterial cells such as Escherichia coli, insect cells, yeast or mammalian cells (such as Chinese hamster ovary cells ((CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX protein or can be introduced on a separate vector. Cells stably-transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a NOVX protein. Accordingly, the invention further provides methods for producing NOVX proteins using the host cells of the invention. In one embodiment, the method comprises culturing the host cell of invention (i.e., into which a recombinant expression vector encoding a NOVX protein has been introduced) in a suitable medium such that the NOVX protein is produced. In another embodiment, the method further comprises isolating the protein from the medium or the host cell.

### Transgenic Animals

The host cells of the invention can also be used to produce non-human transgenic animals. For example, in one embodiment, a host cell of the invention is a fertilized oocyte or an embryonic stem cell into which NOVX protein-coding sequences have been introduced. These host cells can then be used to create non-human transgenic animals in which exogenous NOVX nucleic acids sequences have been introduced into their genome or homologous recombinant animals in which endogenous NOVX sequences have been altered. Such animals are useful for studying the function and/or activity of NOVX protein and for identifying and/or evaluating modulators of the protein's activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA that is integrated into the genome of a cell from which a transgenic animal develops and that remains in the genome of the mature animal, thereby directing the expression of an encoded gene product in one or more cell types, e.g. liver, or tissues of the transgenic animal. As used herein, a “homologous recombinant animal” is a non-human animal, preferably a mammal, more preferably a mouse, in which an endogenous NOVX protein gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX protein-encoding nucleic acid into the male pronuclei of a fertilized oocyte (e.g., by micro-injection, retroviral infection) and allowing the oocyte to develop in a pseudopregnant female foster animal. The NOVX protein DNA sequence, e.g., one of SEQ ID NOs: 1, 3, 5, 7, 9, or 11, can be introduced as a transgene into the genome of a non-human animal. Alternatively, a non-human homologue of the NOVX protein gene, such as a mouse NOVX protein gene, can be isolated based on hybridization to the human gene DNA and used as a transgene. Intronic sequences and polyadenylation signals can also be included in the transgene to increase the efficiency of expression of the transgene. A tissue-specific regulatory sequence(s) can be operably-linked to the NOVX protein transgene to direct expression of the protein to particular cells, e.g. liver cells. Methods for generating transgenic animals via embryo manipulation and micro-injection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX protein transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX protein gene into which a deletion, addition or substitution has been introduced to thereby alter, e.g., functionally disrupt, the NOVX gene. The NOVX protein gene can be a human gene (e.g., one of SEQ ID NOs: 1, 3, 5, 7, 9, or 11), but more preferably is a non-human homolog of a NOVX protein gene. For example, a mouse homologue of a NOVX protein gene can be used to construct a homologous recombination vector suitable for altering an endogenous NOVX protein gene in the mouse genome. In one embodiment, the vector is designed such that, upon homologous recombination, the endogenous NOVX protein gene is functionally disrupted (i. e., no longer encodes a functional protein; also referred to as a “knock out” vector).

Alternatively, the vector can be designed such that, upon homologous recombination, the endogenous NOVX protein gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX protein nucleic acid is of sufficient length for successful homologous recombination with the endogenous gene. Typically, several kilobases (Kb) of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then micro-injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

### Pharmaceutical Compositions

The NOVX nucleic acid molecules, NOVX proteins, and anti-NOVX protein antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically-acceptable carrier. As used herein, “pharmaceutically-acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and other non-aqueous (i.e., lipophilic) vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX protein antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Screening and Detection Methods

The nucleic acid molecules, proteins, protein homologues, and antibodies described herein can be used in one or more of the following methods: (A) screening assays; (B) detection assays (e.g., chromosomal mapping, cell and tissue typing, forensic biology), (C) predictive medicine (e.g., diagnostic assays, prognostic assays, monitoring clinical trials, and pharmacogenomics); and (D) methods of treatment (e.g., therapeutic and prophylactic).

The isolated nucleic acid molecules of the present invention can be used to express NOVX proteins (e.g., via a recombinant expression vector in a host cell in gene therapy applications), to detect NOVX mRNAs (e.g., in a biological sample) or a genetic lesion in a NOVX protein gene, and to modulate a NOVX protein activity, as described further, below. In addition, the NOVX proteins can be used to screen drugs or compounds that modulate the protein activity or expression as well as to treat disorders characterized by insufficient or excessive production of a NOVX protein or production of NOVX protein forms that have decreased or aberrant activity compared to wild-type protein. In addition, the anti-NOVX protein antibodies of the present invention can be used to detect and isolate proteins and modulate NOVX protein activity. In addition to the use of NOVX nucleic acids and proteins in these methods, NOVINTRA C (human IL-1 epsilon) nucleic acid sequences and protein may be used as described below to treat disorders and diseases which, in accordance with the invention, involve differential expression of human IL-1 epsilon; e.g. lung cancer and disorders of the airways, such as asthma.

The invention further pertains to novel agents identified by the screening assays described herein and uses thereof for treatments as described, above.

### Screening Assays

The invention provides a method (also referred to herein as a “screening assay”) for identifying modulators, i.e., candidate or test compounds or agents (e.g., peptides, peptidomimetics, small molecules or other drugs) that bind to NOVX protein or have a stimulatory or inhibitory effect on, e.g., NOVX protein expression or NOVX protein activity, e.g. in liver cells. The invention also includes compounds identified in the screening assays described herein.

In one embodiment, the invention provides assays for screening candidate or test compounds which bind to or modulate the activity of the membrane-bound form of a NOVX protein or polypeptide, e.g. NOVTRAN, or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead, one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can be of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX protein to form an assay mixture, contacting the assay mixture with a test compound, and determining the ability of the test compound to interact with a NOVX protein, wherein determining the ability of the test compound to interact with the protein comprises determining the ability of the test compound to preferentially bind to the NOVX protein or a biologically-active portion thereof as compared to the known compound.

In another embodiment, an assay is a cell-based assay comprising contacting a cell expressing a membrane-bound form of a NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of a NOVX protein or a biologically-active portion thereof can be accomplished, for example, by determining the ability of the protein to bind to or interact with a NOVX protein target molecule. As used herein, a “target molecule” is a molecule with which the NOVX protein binds or interacts in nature, for example, a molecule on the surface of a cell which expresses a NOVX protein interacting protein, a molecule on the surface of a second cell, a molecule in the extracellular milieu, a molecule associated with the internal surface of a cell membrane or a cytoplasmic molecule. An NOVX protein target molecule can be a non-NOVX molecule or a NOVX protein or polypeptide of the invention. In one embodiment, a NOVX protein target molecule is a component of a signal transduction pathway that facilitates transduction of an extracellular signal (e.g. a signal generated by binding of a compound to a membrane-bound NOVX protein molecule) through the cell membrane and into the cell. The target, for example, can be a second intercellular protein that has catalytic activity or a protein that facilitates the association of downstream signaling molecules with a NOVX protein.

Determining the ability of the NOVX protein to bind to or interact with a NOVX protein target molecule can be accomplished by one of the methods described above for determining direct binding. In one embodiment, determining the ability of the NOVX protein to bind to or interact with a NOVX protein target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX protein-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds the protein or portion to form an assay mixture, contacting the assay mixture with a test compound, and determining the ability of the test compound to interact with a NOVX protein, wherein determining the ability of the test compound to interact with the protein comprises determining the ability of the test compound to preferentially bind to NOVX protein or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX protein can be accomplished, for example, by determining the ability of the protein to bind to a NOVX protein target molecule by one of the methods described above for determining direct binding. In an alternative embodiment, determining the ability of the test compound to modulate the activity of NOVX protein can be accomplished by determining the ability of the protein to further modulate a NOVX protein target molecule. For example, the catalytic/enzymatic activity of the target molecule ion an appropriate substrate can be determined as described, above.

In yet another embodiment, the cell-free assay comprises contacting the NOVX protein or biologically-active portion thereof with a known compound which binds the NOVX protein to form an assay mixture, contacting the assay mixture with a test compound, and determining the ability of the test compound to interact with NOVX protein, wherein determining the ability of the test compound to interact with the protein comprises determining the ability of the NOVX protein to preferentially bind to or modulate the activity of a NOVX protein target molecule.

The cell-free assays of the invention are amenable to use of both the soluble form or the membrane-bound form of NOVX proteins. In the case of cell-free assays comprising the membrane-bound form of the protein, it may be desirable to utilize a solubilizing agent such that the membrane-bound form of a NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either the a NOVX protein or its target molecule to facilitate separation of complexed from non-complexed forms of one or both of the proteins, as well as to accommodate automation of the assay. Binding of a test compound to a NOVX protein, or interaction of a NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, above. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with a NOVX protein or target molecules, but which do not interfere with binding of the NOVX protein to its target molecule, can be derivatized to the wells of the plate, and unbound target or the NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of a NOVX protein mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX protein mRNA or protein expression. Alternatively, when expression of the mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, NOVX proteins can be used as a “bait protein” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with a NOVX protein (“NOVX protein-binding proteins” or “NOVX protein-bp”) and modulate its activity. Such NOVX protein-binding proteins are also likely to be involved in the propagation of signals by NOVX protein as, for example, upstream or downstream elements of the NOVX protein pathway.

The two-hybrid system is based on the modular nature of most transcription factors, which consist of separable DNA-binding and activation domains. Briefly, the assay utilizes two different DNA constructs. In one construct, the gene that codes for NOVX protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX protein-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX protein.

The invention further pertains to novel agents identified by the aforementioned screening assays and uses thereof for treatments as described herein.

### Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

### Chromosome Mapping

Once the sequence (or a portion of the sequence) of a gene has been isolated, this sequence can be used to map the location of the gene on a chromosome. This process is called chromosome mapping. Accordingly, portions or fragments of a NOVX nucleic acid sequence, e.g. a portion or fragment of one of SEQ ID NOs: 1, 3, 5, 7, 9, or 11, or fragments or derivatives thereof, can be used to map the location of the NOVX gene on a chromosome. The mapping of the NOVX sequence to chromosomes is an important first step in correlating this sequence with genes associated with disease.

Briefly, a NOVX gene can be mapped to chromosomes by preparing PCR primers (preferably 15-25 bp in length) from the NOVX sequence. Computer analysis of the NOVX sequence can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX nucleic acid sequence will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequence to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Additionally, differences in the DNA sequences between individuals affected and unaffected with a disease associated with the NOVX gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The NOVX nucleic acid sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” as described in U.S. Pat. No. 5,272,057).

Furthermore, the sequences of the invention can be used to provide an alternative technique that determines the actual base-by-base DNA sequence of selected portions of an individual's genome. Thus, the NOVX nucleic acid sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences can comfortably provide positive individual identification with a panel of perhaps 10 to 1,000 primers that each yield a non-coding amplified sequence of 100 bases. If predicted NOVX protein coding sequences, e.g. one of SEQ ID NOs: 1, 3, 5, 7, 9, or 11, are used, a more appropriate number of primers for positive individual identification would be 500-2,000.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX protein activity, in the context of a biological sample (e.g., blood, serum, cells, tissue) to thereby determine whether an individual is afflicted with a disease or disorder, or is at risk of developing a disorder, associated with aberrant NOVX protein expression or activity. The invention also provides for prognostic (or predictive) assays for determining whether an individual is at risk of developing a disorder associated with a NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with a NOVX protein, nucleic acid expression or activity. For example, the disease associated with aberrant expression of a NOVINTRA protein may be an immune or inflammatory disorder, e.g. septic shock or arthritis, as described above.

Another aspect of the invention provides methods for determining NOVX nucleic acid expression or NOVX protein activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of a NOVX protein in clinical trials.

In addition to the use of NOVX nucleic acids and proteins in these methods, NOVINTRA C (human IL-1 epsilon) nucleic acid sequences and protein may be used as described below to treat disorders and diseases which, in accordance with the invention, have been discovered to involve differential expression of human IL-1 epsilon; e.g. lung cancer and disorders of the airways, such as asthma.

These and other agents are described in further detail in the following sections.

### Diagnostic Assays

An exemplary method for detecting the presence or absence of a NOVX protein in a biological sample involves obtaining a biological sample from a test subject and contacting the biological sample with a compound or an agent capable of detecting the NOVX protein or nucleic acid (e.g., mRNA, genomic DNA) that encodes the NOVX protein such that the presence of the NOVX protein or nucleic acid is detected in the biological sample. An agent for detecting a NOVX mRNA or genomic DNA is a labeled nucleic acid probe capable of hybridizing to a NOVX mRNA or genomic DNA. The nucleic acid probe can be, for example, a full-length NOVX nucleic acid, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting a NOVX protein is an antibody capable of binding to the NOVX protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of a NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of a NOVX protein include introducing into a subject a labeled anti-NOVX protein antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a NOVX protein, mRNA, or genomic DNA, such that the presence of the NOVX protein, mRNA or genomic DNA is detected in the biological sample, and comparing the presence of a NOVX protein, mRNA or genomic DNA in the control sample with the presence of the NOVX protein, mRNA or genomic DNA in the test sample.

The invention also encompasses kits for detecting the presence of a NOVX protein in a biological sample. For example, the kit can comprise: a labeled compound or agent capable of detecting NOVX protein or mRNA in a biological sample; means for determining the amount of NOVX protein or mRNA in the sample; and means for comparing the amount of the NOVX protein in the sample with a standard. The compound or agent can be packaged in a suitable container. The kit can further comprise instructions for using the kit to detect a NOVX protein or nucleic acid.

### Prognostic Assays

The diagnostic methods described herein can furthermore be utilized to identify subjects having or at risk of developing a disease or disorder associated with aberrant NOVX protein expression or activity. For example, the assays described herein, such as the preceding diagnostic assays or the following assays, can be utilized to identify a subject having or at risk of developing a disorder associated with a NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX protein expression or activity in which a test sample is obtained from a subject and NOVX protein or nucleic acid (e.g., mRNA, genomic DNA) is detected, wherein the presence of the NOVX protein or nucleic acid is diagnostic for a subject having or at risk of developing a disease or disorder associated with aberrant NOVX protein expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX protein expression or activity, e.g. aberrant immune or inflammatory response associated with aberrant NOVINTRA expression or activity. For example, such methods can be used to determine whether a subject can be effectively treated with an agent for a disorder. Thus, the invention provides methods for determining whether a subject can be effectively treated with an agent for a disorder associated with aberrant NOVX protein expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX protein expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding NOVX protein, or the mis-expression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX protein gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX protein gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to the NOVX gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX sequences can be identified in two dimensional arrays containing light-generated DNA probes as described in Cronin, et al., above. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX gene with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX protein gene include methods in which protection from cleavage agents is used to detect mismatched bases in RNA/RNA or RNA/DNA heteroduplexes. See, e.g., Myers, et al., 1985. Science 230:1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX nucleic acid sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in a NOVX gene. For example, single-strand conformation polymorphism (SSP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′- terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sc: USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX protein gene.

Furthermore, any cell type or tissue, preferably liver cells, in which a NOVX protein is expressed may be utilized in the prognostic assays described herein. However, any biological sample containing nucleated cells may be used, including, for example, buccal mucosal cells.

In all of the above-identified methods, differential expression of NOVINTRA C (human IL-1 epsilon) nucleic acid sequences and protein associated with lung cancer and small airway epithelium provides for novel methods of diagnosing and treating these disorders, e.g. asthma. For example, a monoclonal antibody directed against the NOVINTRA C protein could be used as a diagnostic tool for a subset of lung cancer, and as a therapeutic tool to treat lung cancer in a set of patients including those having this type of lung cancer. Similarly, IL-1 epsilon expression provides for diagnosis of diseases such as asthma, irritation in the lungs due to allergies, and inflammatory conditions in diseases such as emphysema. Currently, no diagnostic test exists for the presence of IL-1 Epsilon (NOVINTRA C) in the lungs of asthmatic patients or those suffering from irritation of the airways due to allergies. Providing an accurate indicator of the presence and measurement of the amount of IL-1 Epsilon may assist in the diagnosis and treatment of asthmatic and allergy patients. NOVINTRA C (IL-1 Epsilon), could thus be used as a monoclonal antibody target in Enzyme Linked Immunosorbent Assays (ELISA) to provide a means of detection for IL-1 Epsilon.; bronchoalveolar or nasal lavage (BAL, NL) fluid from allergy and asthmatic patients may be obtained and assayed in ELISA experiments to quantify the relative amount of IL-1 Epsilon. This diagnostic tool should allow for more efficient identification and alleviation of symptoms of inflammation in asthma and allergy patients.

### Pharmacogenomics

Agents, or modulators that have a stimulatory or inhibitory effect on NOVX protein activity (e.g., NOVX protein gene expression), as identified by a screening assay described herein can be administered to individuals to treat (prophylactically or therapeutically) disorders associated with aberrant NOVX protein activity. For example, the disorder can be a cell signaling disorder, e.g. cancer, associated with aberrant NOVTRAN activity.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX proteins, expression of NOVX nucleic acids, or mutation content of a NOVX protein genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so-called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX protein nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX protein modulator, such as a modulator identified by one of the exemplary screening assays described herein.

### Monitoring of Effects During Clinical Trials

Monitoring the influence of agents (e.g., drugs, compounds) on the expression or activity of a NOVX protein (e.g., for NOVINTRA, the ability to modulate immune and inflammatory responses) can be applied not only in basic drug screening, but also in clinical trials. For example, the effectiveness of an agent determined by a screening assay as described herein to increase NOVX protein gene expression, protein levels, or upregulate NOVX protein activity, can be monitored in clinical trails of subjects exhibiting decreased NOVX gene expression, protein levels, or down-regulated NOVX protein activity. Alternatively, the effectiveness of an agent determined by a screening assay to decrease NOVX gene expression, protein levels, or down-regulate NOVX protein activity, can be monitored in clinical trails of subjects exhibiting increased NOVX gene expression, protein levels, or up-regulated NOVX protein activity. In such clinical trials, the expression or activity of NOVX protein and, preferably, other genes that have been implicated in, for example, metabolic disorders, can be used as a “read out” or markers of the metabolic responsiveness of a particular cell.

By way of example, and not of limitation, genes including NOVX protein genes, that are modulated in cells by treatment with an agent (e.g., compound, drug or small molecule) that modulates NOVX protein activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX protein and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX protein or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of NOVX protein, mRNA, or genomic DNA in the pre-administration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of a NOVX protein to higher levels than detected, i.e., to increase the effectiveness of the agent. Alternatively, decreased administration of the agent may be desirable to decrease expression or activity of the NOVX protein to lower levels than detected, i.e., to decrease the effectiveness of the agent.

### Methods of Treatment

The invention provides for both prophylactic and therapeutic methods of treating a subject at risk of (or susceptible to) a disorder or having a disorder associated with aberrant NOVX protein expression or activity. These methods of treatment will be discussed more fully below.

### Disease and Disorders

Diseases and disorders that are characterized by increased (relative to a subject not suffering from the disease or disorder) levels or biological activity may be treated with therapeutics that antagonize (i.e., reduce or inhibit) activity. For example, as discussed above, disorders associated with transmembrane proteins (e.g. NOVTRAN) include diseases involving altered cell signaling, such as cancer, immune and hematopoietic disorders, or neurodegenerative disease. Disorders associated with neuromedins (e.g. NOVNEUR) include endocrine, muscle, or neurologic diseases, or cancer. Disorders associated with hormonal proteins, like gonadotropins (e.g. NOVGON), include those involving reproductive development, weight gain/loss, metabolic function, or other hormonally-modulated diseases, such as cancer (e.g. Karposi's sarcoma). Disorders associated with interleukin-1 receptor antagonsists (e.g. NOVINTRA A, B and C) include those involving bone metabolism and structure, inflammatory response, and immune regulation, or diseases such as septic shock, stroke, diabetes, arthritis and cancer. Hence, the administration of a modulator of NOVX protein expression or activity provides a means to treat these respective disorders associated with aberrant expression of NOVX proteins.

In another embodiment, diseases of the lung now discovered to be associated with differential expression of IL-1 epsilon (e.g. NOVINTRA C) may be treated by administration of modulators of such expression. For example, a monoclonal antibody directed against the IL-1 epsilon could be used as a therapeutic tool to treat lung cancer, asthma, irritation in the lungs due to allergies, and inflammatory conditions in diseases such as emphysema.

Therapeutics that antagonize activity may be administered in a therapeutic or prophylactic manner. Therapeutics that may be utilized include, but are not limited to: (i) an aforementioned NOVX protein, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244:1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Prophylactic Methods

In one aspect, the invention provides a method for preventing, in a subject, a disease or condition associated with an aberrant NOVX protein expression or activity, by administering to the subject an agent that modulates the NOVX protein expression or at least one NOVX protein activity. Subjects at risk for a disease, e.g. an inflammatory response disorder, that is caused or contributed to by aberrant NOVX protein expression or activity can be identified by, for example, any or a combination of diagnostic or prognostic assays as described herein. Administration of a prophylactic agent can occur prior to the manifestation of symptoms characteristic of the NOVX protein aberrancy, such that a disease or disorder is prevented or, alternatively, delayed in its progression. Depending upon the type of NOVX protein aberrancy, for example, a NOVX protein agonist or NOVX protein antagonist agent can be used for treating the subject. For example, aberrant immune or inflammatory response metabolism may be either upregulated or down regulated by administering the appropriate NOVX protein modulator, e.g. a modulator of NOVINTRA activity, to a subject. The appropriate agent can be determined based on screening assays described herein.

### Therapeutic Methods

Another aspect of the invention pertains to methods of modulating NOVX protein expression or activity for therapeutic purposes. The modulatory method of the invention involves contacting a cell with an agent that modulates one or more of the activities of NOVX protein activity associated with the cell. An agent that modulates NOVX protein activity can be an agent as described herein or a nucleic acid or a protein, a naturally-occurring cognate ligand of NOVX protein, a peptide, a NOVX protein peptidomimetic, or other small molecule. In one embodiment, the agent stimulates one or more NOVX protein activity. Examples of such stimulatory agents include active NOVX protein and a nucleic acid molecule encoding NOVX protein that has been introduced into the cell. In another embodiment, the agent inhibits one or more NOVX protein activities. Examples of such inhibitory agents include antisense NOVX nucleic acid molecules and anti-NOVX protein antibodies. These modulatory methods can be performed in vitro (e.g., by culturing the cell with the agent) or, alternatively, in vivo (e.g., by administering the agent to a subject). As such, the invention provides methods of treating a disease or disorder in a subject, e.g. a mammal, characterized by aberrant expression or activity of NOVX protein or nucleic acid molecule. In one embodiment, the method involves administering an agent (e.g., an agent identified by a screening assay described herein), or combination of agents that modulates (e.g., up-regulates or down-regulates) NOVX protein expression or activity. In another embodiment, the method involves administering NOVX protein or nucleic acid molecule as therapy to compensate for reduced or aberrant NOVX protein expression or activity.

Both the novel nucleic acids encoding NOVX proteins, and the NOVX proteins of the invention, or fragments thereof, may also be useful in diagnostic applications, wherein the presence or amount of the nucleic acid or the protein are to be assessed. For example, NOVNEUR is expressed in several cancer cell lines, including CNS cancers, lung cancer (non small cell), breast cancer, colon cancer, ovarian cancer, kidney cancer, prostate cancer, thyroid cancer, and lung cancer. Accordingly, by way of example, NOVNEUR nucleic acid may be used as a specific diagnostic probe for these types of cancer, and NOVNEUR protein may serve as a target for an antibody or for a small molecule drug in the treatment of these cancers. Similarly, by way of example, NOVGON is highly expressed in certain normal tissues and in a melanoma cell line. Accordingly, NOVGON nucleic acid may serve as a diagnostic probe for certain specific cancer types, and NOVGON protein may serve as a target for the treatment of certain cancer, e.g. melanoma.

In another embodiment, NOVINTRA C (IL-1 Epsilon) differential expression (e.g. in treated small airway epithelium and lung cancer tissue) provides a diagnostic tool for patients at risk of asthma, irritation of the airways due to allergies, emphysema, and/or lung cancer, or treatment of the same. The expression profile of NOVINTRA C (IL-1 Epsilon) has demonstrated that it has disease association with asthma, allergy and emphysema. It may play a potential role in the development of these diseases. Therefore it has potential usefulness as a therapeutic target, for example, as a target for an IL-1 Epsilon-specific monoclonal antibody, other protein therapeutic or small molecule therapeutic.

These materials are further useful in the generation of antibodies that immunospecifically-bind to the novel substances of the invention for use in therapeutic or diagnostic methods.

### Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### EXAMPLE 1

### Real Time Quantitative (RTQ) PCR Evaluation of Expression of NOVX Clones in Various Cells and Tissues

The quantitative expression of various NOVX clones was assessed in normal and tumor samples by real time quantitative PCR (TAQMAN®) performed on a Perkin-Elmer Biosystems ABI PRISM® 7700 Sequence Detection System. In the Tables in this Example the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

First, 96 RNA samples were normalized to internal standards such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using internal standards such as β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin /GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tₘ=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions were as follows: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCT2, dNTPs (dA, G, C, U at 1:1:1:2 ratios), 0.25 U/ml AmpliTaq Gold™ (PE Biosystems), and 0.4 U/β1 RNase inhibitor, and 0.25 U/β1 reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

Two sample panels are employed in the present Example. Panel 1 is a 96 well plate (usually 2 control wells and 94 test samples) whose wells are contain RNA or cDNA isolated from various human cell lines that have been established from human malignant tissues (i.e., tumors). These cell lines have been extensively characterized by investigators in both academia and the commercial sector regarding their tumorgenicity, metastatic potential, drug resistance, invasive potential and other cancer-related properties. They serve as suitable tools for pre-clinical evaluation of anti-cancer agents and promising therapeutic strategies. RNA from these various human cancer cell lines was isolated by and procured from the Developmental Therapeutic Branch (DTB) of the National Cancer Institute (USA). Basic information regarding their biological behavior, gene expression, and resistance to various cytotoxic agents are provided by the DTB (http://dtp.nci.nih.gov/).

In addition, RNA or cDNA was obtained from various human tissues derived from human autopsies performed on deceased elderly people or sudden death victims (accidents, etc.). These tissues were ascertained to be free of disease and were purchased from various high quality commercial sources such as Clontech, Research Genetics, and Invitrogen.

RNA integrity from all samples is controlled for quality by visual assessment of agarose gel electrophoresis using 28s and 18s ribosomal RNA staining intensity ratio as a guide (2:1 to 2.5:1 28s:18s) and the assuring the absence of low molecular weight RNAs indicative of degradation products.

Panel 2 is a 96 well plate (usually 2 control wells and 94 test samples) containing RNA or cDNA isolated from human tissue procured by surgeons working in close cooperation with the National Cancer Institute's Cooperative Human Tissue Network (CHTN) or the National Disease Research Initiative (NDRI). The tissues procured are derived from human malignancies and in cases where indicated many malignant tissues have “matched margins”. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA or cDNA was obtained from various human tissues derived from human autopsies performed on deceased elderly people or sudden death victims (accidents, etc.). These tissue were ascertained to be free of disease and were purchased from various high quality commercial sources such as Clontech, Research Genetics, and Invitrogen.

RNA integrity from all samples is controlled for quality by visual assessment of agarose gel electrophoresis using 28S and 18S ribosomal RNA staining intensity ratio as a guide (2:1 to 2.5:1 28s:18s) and by assuring the absence of low molecular weight RNAs indicative of degradation products. Samples are quality controlled for genomic DNA contamination by reactions run in the absence of reverse transcriptase using probe and primer sets designed to amplify across the span of a single exon.

A) RTQPCR of NOVGON Nucleic Acid

RTQ PCR of the NOVGON nucleic acid sequence described herein (SEQ ID NO: 5; see also FIG. 6A) was carried out on Panel 1, as shown in Table 2, below, using the primer-probe set Ag338 designated in Table 1, below.

TABLE 1
Primer-probe set Ag338 for NOVGON nucleic acid sequence

| | | | Start |
|---|---|---|---|
| Primers | Sequences | Length | Position |
| Forward | 5′-ACAACGAGACCAAACAGGTGACT-3 | 23 | 134 |
| | (SEQ ID NO:13) | | |
| Probe | TET-5′-TCAAGCTGCCCAACTGTGCCCC-3′-TAMRA | 22 | 158 |
| | (SEQ ID NO:14) | | |
| Reverse | 5′-GGCCACGGGATAGGTGTAGA-3′ | 20 | 194 |
| | (SEQ ID NO:15) | | |

TABLE 2
RTQ PCR results

| | Rel. | | Rel. |
|---|---|---|---|
| Cell source | Expr., % | Cell source | Expr., % |
| Endothelial cells | 0.0 | Renal ca. 786-0 | 0.0 |
| Endothelial cells (treated) | 0.0 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adipose | 100.0 | Renal ca. UO-31 | 0.0 |
| Adrenal gland | 0.0 | Renal ca. TK-10 | 0.0 |
| Thyroid | 0.0 | Liver | 0.0 |
| Salivary gland | 0.0 | Liver (fetal) | 0.0 |
| Pituitary gland | 0.0 | Liver ca. (hepatoblast) HepG2 | 0.0 |
| Brain (fetal) | 0.0 | Lung | 0.0 |
| Brain (whole) | 0.3 | Lung (fetal) | 0.0 |
| Brain (amygdala) | 0.4 | Lung ca. (small cell) LX-1 | 0.0 |
| Brain (cerebellum) | 0.2 | Lung ca. (small cell) NCI-H69 | 5.0 |
| Brain (hippocampus) | 0.0 | Lung ca. (s.cell var.) SHP-77 | 0.0 |
| Brain (substantia nigra) | 0.0 | Lung ca. (large cell) NCI-H460 | 0.0 |
| Brain (thalamus) | 0.1 | Lung ca. (non-sm. cell) A549 | 0.2 |
| Brain (hypothalamus) | 0.0 | Lung ca. (non-s.cell) NCI-H23 | 0.0 |
| Spinal cord | 0.0 | Lung ca (non-s.cell) HOP-62 | 0.0 |
| CNS ca. (glio/astro) U87-MG | 0.0 | Lung ca. (non-s.cl) NCI-H522 | 0.0 |
| CNS ca. (glio/astro) U-118-MG | 0.0 | Lung ca. (squam.) SW 900 | 0.0 |
| CNS ca. (astro) SW1783 | 0.0 | Lung ca. (squam.) NCI-H596 | 1.1 |
| CNS ca.* (neuro; met) SK-N-AS | 0.0 | Mammary gland | 0.0 |
| CNS cs. (astro) SF-539 | 0.0 | Breast ca.* (pl. effusion) MCF- | 0.0 |
| | | 7 | |
| CNS ca. (astro) SNB-75 | 0.0 | Breast ca.* (pl.ef) MDA-MB- | 0.0 |
| | | 231 | |
| CNS ca. (glio) SNB-19 | 0.0 | Breast ca.* (pl. effusion) T47D | 0.6 |
| CNS ca. (glio) U251 | 0.0 | Breast ca. BT-549 | 0.0 |
| CNS Ca. (glio) SF-295 | 0.0 | Breast ca. MDA-N | 0.0 |
| Heart | 0.0 | Ovary | 0.0 |
| Skeletal muscle | 0.0 | Ovarian ca. OVCAR-3 | 0.0 |
| Bone marrow | 0.0 | Ovarian ca. OVCAR-4 | 0.0 |
| Thymus | 0.0 | Ovarian ca. OVCAR-5 | 6.2 |
| Spleen | 0.0 | Ovarian ca. OVCAR-8 | 0.0 |
| Lymph node | 0.0 | Ovarian ca. IGROV-1 | 0.9 |
| Colon (ascending) | 13.9 | Ovarian ca.* (ascites) SK-OV-3 | 0.0 |
| Stomach | 0.0 | Uterus | 0.0 |
| Small intestine | 0.0 | Placenta | 0.0 |
| Colon ca. SW480 | 0.0 | Prostate | 0.0 |
| Colon ca.* (SW480 met)SW620 | 0.0 | Prostate ca.* (bone met) PC-3 | 0.3 |
| Colon ca. HT29 | 0.0 | Testis | 3.5 |
| Colon ca. HCT-116 | 0.0 | Melanoma Hs688(A).T | 0.0 |
| Colon ca. CaCo-2 | 0.0 | Melanoma* (met) Hs688(B).T | 0.0 |
| Colon ca. HCT-15 | 1.0 | Melanoma UACC-62 | 0.0 |
| Colon ca. HCC-2998 | 0.0 | Melanoma M14 | 0.0 |
| Gastric ca.* (liver met) NCI-N87 | 0.0 | Melanoma LOX IMVI | 0.0 |
| Bladder | 0.0 | Melanoma* (met) SK-MEL-5 | 0.0 |
| Trachea | 0.0 | Melanoma SK-MEL-28 | 59.9 |
| Kidney | 0.0 | Melanoma UACC-257 | 0.0 |
| Kidney (fetal) | 0.0 | Renal ca. 786-0 | 0.0 |

The results in Table 2 show that the NOVGON nucleic acid sequence of the invention is highly expressed in certain normal tissues and in a melanoma cell line. This suggests that NOVGON may serve as a diagnostic probe for certain specific cancer types.

B) RTQPCR of NOVNEUR Nucleic Acid

RTQ PCR of the NOVNEUR nucleic acid sequence of the invention (SEQ ID NO: 3; see also FIG. 3A) was carried out on Panel 1, as shown in Table 4, using the primer-probe set Ag235 designated in Table 3, below. Two replicate runs are shown in Table 4.

TABLE 3
Primer-probe set Ag235 for NOVNEUR nucleic acid sequence

| Primers | Sequences | Length |
|---|---|---|
| Forward | 5′-TTCCAGCCCATCCCCATT-3′ | 18 |
| | (SEQ ID NO:16) | |
| Probe | FAM-5′-CCCCACACCTCCCTGAGGGACC-3′-TAMRA | 22 |
| | (SEQ ID NO:17) | |
| Reverse | 5′-CAGATCATGACTCAGCTGCAGTC-3′ | 23 |
| | (SEQ ID NO:18) | |

TABLE 4
RTQ PCR results on Panel 1

| | Rel. | Rel. | | Rel. | Rel. |
|---|---|---|---|---|---|
| | Expr., | Expr., | | Expr., | Expr., |
| | %, | %, | | %, | %, |
| | 1.2tml | 1.2tml | | 1.2tml | 1.2tml |
| Cell source | 024f | 192f | Cell source | 024f | 192f |
| Endothelial cells | 4.6 | 4.0 | Renal ca. 786-0 | 0.3 | 0.2 |
| Endothelial cells (treated) | 0.7 | 1.8 | Renal ca. A498 | 4.3 | 1.5 |
| Pancreas | 20.6 | 0.6 | Renal ca. RXF 393 | 8.7 | 5.3 |
| Pancreatic ca. CAPAN 2 | 4.3 | 2.7 | Renal ca. ACHN | 2.2 | 1.0 |
| Adrenal Gland (new lot*) | 100.0 | 100.0 | Renal ca. UO-31 | 0.6 | 0.3 |
| Thyroid | 72.2 | 6.2 | Renal ca. TK-10 | 1.4 | 0.8 |
| Salavary gland | 12.6 | 13.8 | Liver | 0.4 | 1.5 |
| Pituitary gland | 23.3 | 2.7 | Liver (fetal) | 1.1 | 1.8 |
| Brain (fetal) | 13.7 | 7.8 | Liver ca. (hepatoblast) | 1.7 | 1.4 |
| | | | HepG2 | | |
| Brain (whole) | 11.7 | 12.3 | Lung | 3.4 | 3.3 |
| Brain (amygdala) | 5.7 | 8.3 | Lung (fetal) | 2.9 | 2.0 |
| Brain (cerebellum) | 4.5 | 11.9 | Lung ca. (small cell) LX- | 7.8 | 1.5 |
| | | | 1 | | |
| Brain (hippocampus) | 11.3 | 26.2 | Lung ca. (small cell) | 0.3 | 0.2 |
| | | | NCI-H69 | | |
| Brain (thalamus) | 4.2 | 11.9 | Lung ca. (s.cell var.) | 3.8 | 1.4 |
| | | | SHP-77 | | |
| Cerebral Cortex | 3.9 | 19.5 | Lung ca. (large cell) | 6.0 | 11.8 |
| | | | NCI-H460 | | |
| Spinal cord | 17.4 | 19.5 | Lung ca. (non-sm. cell) | 6.9 | 1.7 |
| | | | A549 | | |
| CNS ca. (glio/astro) U87- | 43.2 | 16.3 | Lung ca. (non-s.cell) | 12.9 | 24.2 |
| MG | | | NCI-H23 | | |
| CNS ca. (glio/astro) U-118- | 1.0 | 0.3 | Lung ca (non-s.cell) | 2.4 | 0.5 |
| MG | | | HOP-62 | | |
| CNS ca. (astro) SW1783 | 1.9 | 0.6 | Lung ca. (non-s.cl) NCI- | 27.0 | 4.0 |
| | | | H522 | | |
| CNS ca.* (neuro; met) SK- | 29.3 | 10.7 | Lung ca. (squam.) SW | 6.0 | 2.4 |
| N-AS | | | 900 | | |
| CNS ca. (astro) SF-539 | 1.2 | 0.4 | Lung ca. (squam.) NCI- | 1.0 | 0.3 |
| | | | H596 | | |
| CNS ca. (astro) SNB-75 | 0.0 | 0.3 | Mammary gland | 39.5 | 20.3 |
| CNS ca. (glio) SNB-19 | 25.2 | 11.8 | Breast ca.* (pl. effusion) | 24.0 | 13.0 |
| | | | MCF-7 | | |
| CNS ca. (glio) U251 | 5.3 | 2.8 | Breast ca.* (pl.ef) MDA- | 0.9 | 0.5 |
| | | | MB-231 | | |
| CNS ca. (glio) SF-295 | 0.2 | 0.1 | Breast ca.* (pl. effusion) | 0.9 | 4.3 |
| | | | T47D | | |
| Heart | 9.9 | 8.5 | Breast ca. BT-549 | 11.5 | 8.5 |
| Skeletal Muscle (new lot*) | 8.7 | 2.7 | Breast ca. MDA-N | 13.7 | 8.9 |
| Bone marrow | 1.3 | 0.7 | Ovary | 5.8 | 1.9 |
| Thymus | 0.4 | 0.3 | Ovarian ca. OVCAR-3 | 2.0 | 0.8 |
| Spleen | 1.8 | 1.1 | Ovarian ca. OVCAR-4 | 1.1 | 0.3 |
| Lymph node | 1.1 | 3.0 | Ovarian ca. OVCAR-5 | 49.0 | 6.5 |
| Colorectal | 0.0 | 0.3 | Ovarian ca. OVCAR-8 | 1.0 | 1.3 |
| Stomach | 2.7 | 2.4 | Ovarian ca. IGROV-1 | 10.7 | 3.7 |
| Small intestine | 1.4 | 1.3 | Ovarian ca.* (ascites) | 2.0 | 1.7 |
| | | | SK-OV-3 | | |
| Colon ca. SW480 | 1.0 | 0.1 | Uterus | 2.9 | 2.1 |
| Colon ca.* (SW480 | 9.2 | 0.8 | Plancenta | 1.8 | 1.1 |
| met) SW620 | | | | | |
| Colon ca. HT29 | 25.4 | 4.7 | Prostate | 4.7 | 4.9 |
| Colon ca. HCT-116 | 4.8 | 2.0 | Prostate ca.* (bone | 2.8 | 1.7 |
| | | | met) PC-3 | | |
| Colon ca. CaCo-2 | 8.0 | 3.7 | Testis | 33.2 | 5.0 |
| 83219 CC Well to Mod Diff | 0.8 | 1.1 | Melanoma Hs688(A).T | 1.5 | 0.2 |
| (ODO3866) | | | | | |
| Colon ca. HCC-2998 | 42.3 | 14.3 | Melanoma* (met) | 1.8 | 0.2 |
| | | | Hs688(B).T | | |
| Gastric ca.* (liver met) | 54.7 | 18.3 | Melanoma UACC-62 | 1.5 | 0.4 |
| NCI-N87 | | | | | |
| Bladder | 11.7 | 3.5 | Melanoma M14 | 5.5 | 2.5 |
| Trachea | 5.6 | 4.0 | Melanoma LOX IMVI | 6.4 | 1.3 |
| Kidney | 6.7 | 9.1 | Melanoma* (met) SK- | 6.9 | 2.0 |
| | | | MEL-5 | | |
| Kidney (fetal) | 6.8 | 6.4 | Adipose | 3.9 | 39.0 |

The expression of the NOVNEUR nucleic acid sequence of the invention was also evaluated using the same primer-probe set, Ag235, on Panel 2. The results for two replicates are shown in Table 5, below.

TABLE 5
RTQ PCR results on Panel 2.

| | Rel. | Rel. | | Rel. | Rel. |
|---|---|---|---|---|---|
| | Expr., | Expr., | | Expr., | Expr., |
| | 2tm515 | 2tm102 | | 2tm515 | 2tm1027 |
| Tissue Source | f | 7f | Tissue Source | f | f |
| 83786 Kidney Ca, | 0.7 | 2.7 | 87492 Ovary Cancer | 0.2 | 10.6 |
| Nuclear grade 2 | | | (OD04768-07) | | |
| (OD04338) | | | | | |
| 83219 CC Well to Mod | 0.0 | 0.0 | 87493 Ovary NAT | 0.1 | 0.7 |
| Diff (ODO3866) | | | (OD04768-08) | | |
| 83220 CC NAT | 0.0 | 0.2 | Bladder Cancer | 3.3 | 4.6 |
| (ODO3866) | | | INVITROGEN A302173 | | |
| 83221 CC Gr.2 | 0.0 | 0.2 | Bladder Cancer Research | 0.1 | 0.7 |
| rectosigmoid (ODO3868) | | | Genetics RNA 1023 | | |
| 83222 CC NAT | 0.0 | 0.1 | Breast Cancer Clontech | 0.0 | 0.2 |
| (ODO3868) | | | 9100266 | | |
| 83235 CC Mod Diff | 7.9 | 11.0 | Breast Cancer | 0.0 | 0.4 |
| (ODO3920) | | | INVITROGEN A209073 | | |
| 83236 CC NAT | 0.0 | 0.4 | Breast Cancer Res. Gen. | 0.0 | 0.4 |
| (ODO3920) | | | 1024 | | |
| 83237 CC Gr.2 ascend | 0.8 | 1.2 | Breast NAT Clontech | 0.0 | 0.0 |
| colon (ODO3921) | | | 9100265 | | |
| 83238 CC NAT | 0.0 | 0.0 | Breast NAT | 0.0 | 0.2 |
| (ODO3921) | | | INVITROGEN | | |
| | | | A2090734 | | |
| 83239 Lung Met to | 0.0 | 1.4 | GENPAK Breast Cancer | 2.2 | 4.8 |
| Muscle (ODO4286) | | | 064006 | | |
| 83240 Muscle NAT | 1.3 | 0.5 | Gastric Cancer Clontech | 0.1 | 0.4 |
| (ODO4286) | | | 9060395 | | |
| 83241 CC from Partial | 0.0 | 1.9 | Gastric Cancer Clontech | 3.2 | 3.1 |
| Hepatectomy | | | 9060397 | | |
| (ODO4309) | | | | | |
| 83242 Liver NAT | 1.2 | 1.8 | Gastric Cancer GENPAK | 6.0 | 1.1 |
| (ODO4309) | | | 064005 | | |
| 83255 Ocular Mel Met | 1.0 | 3.7 | Kidney Cancer Clontech | 0.0 | 0.1 |
| to Liver (ODO4310) | | | 8120607 | | |
| 83256 Liver NAT | 0.1 | 1.1 | Kidney Cancer Clontech | 1.0 | 4.9 |
| (ODO4310) | | | 8120613 | | |
| 83787 Kidney NAT | 0.0 | 1.7 | Kidney Cancer Clontech | 25.2 | 39.5 |
| (OD04338) | | | 9010320 | | |
| 83788 Kidney Ca | 2.8 | 16.7 | Kidney NAT Clontech | 0.3 | 5.9 |
| Nuclear grade 1/2 | | | 8120608 | | |
| (OD04339) | | | | | |
| 83789 Kidney NAT | 0.2 | 11.5 | Kidney NAT Clontech | 0.0 | 5.3 |
| (OD04339) | | | 8120614 | | |
| 83790 Kidney Ca, Clear | 12.2 | 23.8 | Kidney NAT Clontech | 1.0 | 4.8 |
| cell type (OD04340) | | | 9010321 | | |
| 83791 Kidney NAT | 0.1 | 3.0 | Liver Cancer GENPAK | 0.0 | 1.5 |
| (OD04340) | | | 064003 | | |
| 83792 Kidney Ca, | 2.8 | 9.8 | Liver Cancer Research | 0.0 | 1.3 |
| Nuclear grade 3 | | | Genetics RNA 1025 | | |
| (OD04348) | | | | | |
| 83793 Kidney NAT | 0.2 | 4.1 | Liver Cancer Research | 0.0 | 1.1 |
| (OD04348) | | | Genetics RNA 1026 | | |
| 84136 Lung Malignant | 0.0 | 1.6 | NAT Stomach Clontech | 0.5 | 0.2 |
| Cancer (OD03126) | | | 9060359 | | |
| 84137 Lung NAT | 1.8 | 1.3 | NAT Stomach Clontech | 0.6 | 1.6 |
| (OD03126) | | | 9060394 | | |
| 84138 Lung NAT | 0.0 | 0.5 | NAT Stomach Clontech | 1.2 | 0.9 |
| (OD04321) | | | 9060396 | | |
| 84139 Melanoma Mets to | 1.0 | 4.8 | Normal Bladder | 0.2 | 2.2 |
| Lung (OD04321) | | | GENPAK 061001 | | |
| 84140 Prostate Cancer | 1.0 | 3.0 | Normal Breast GENPAK | 0.0 | 0.5 |
| (OD04410) | | | 061019 | | |
| 84141 Prostate NAT | 0.5 | 1.3 | Normal Colon GENPAK | 0.0 | 0.9 |
| (OD04410) | | | 061003 | | |
| 84871 Lung Cancer | 3.2 | 14.4 | Normal Kidney | 0.0 | 1.3 |
| (OD04404) | | | GENPAK 061008 | | |
| 84872 Lung NAT | 0.0 | 0.0 | Normal Liver GENPAK | 0.6 | 1.5 |
| (OD04404) | | | 061009 | | |
| 84875 Lung Cancer | 29.3 | 43.8 | Normal Lung GENPAK | 0.6 | 0.4 |
| (OD04565) | | | 061010 | | |
| 84877 Breast Cancer | 0.1 | 0.0 | Normal Ovary Res. Gen. | 0.0 | 3.4 |
| (OD04566) | | | | | |
| 85950 Lung Cancer | 0.3 | 7.5 | Normal Prostate | 0.0 | 0.3 |
| (OD04237-01) | | | Clontech A+ 6546-1 | | |
| 85970 Lung NAT | 0.5 | 1.0 | Normal Stomach | 0.0 | 0.6 |
| (OD04237-02) | | | GENPAK 061017 | | |
| 85973 Kidney Cancer | 0.5 | 5.6 | Normal Thyroid | 3.2 | 3.3 |
| (OD04450-01) | | | Clontech A+ 6570-1** | | |
| 85974 Kidney NAT | 0.2 | 3.8 | Normal Uterus GENPAK | 0.0 | 0.3 |
| (OD04450-03) | | | 061018 | | |
| 85975 Breast Cancer | 0.5 | 1.0 | Ovarian Cancer | 0.0 | 3.1 |
| (OD04590-01) | | | GENPAK 064008 | | |
| 85976 Breast Cancer | 0.1 | 4.5 | Paired Liver Cancer | 0.0 | 0.4 |
| Mets (OD04590-03) | | | Tissue Research Genetics | | |
| | | | RNA 6004-T | | |
| 87070 Breast Cancer | 1.2 | 4.7 | Paired Liver Cancer | 0.0 | 1.4 |
| Metastasis | | | Tissue Research Genetics | | |
| (OD04655-05) | | | RNA 6005-T | | |
| 87071 Bladder Cancer | 5.1 | 7.1 | Paired Liver Tissue | 1.4 | 11.3 |
| (OD04718-01) | | | Research Genetics RNA | | |
| | | | 6004-N | | |
| 87072 Bladder Normal | 0.8 | 2.3 | Paired Liver Tissue | 0.0 | 1.6 |
| Adjacent (OD04718-03) | | | Research Genetics RNA | | |
| | | | 6005-N | | |
| 87073 Prostate Cancer | 15.6 | 17.6 | Thyroid Cancer | 0.6 | 5.2 |
| (OD04720-01) | | | GENPAK 064010 | | |
| 87074 Prostate NAT | 0.3 | 1.0 | Thyroid Cancer | 26.1 | 34.4 |
| (OD04720-02) | | | INVITROGEN A302152 | | |
| 87472 Colon mets to | 0.0 | 1.6 | Thyroid NAT | 7.3 | 15.0 |
| lung (OD04451-01) | | | INVITROGEN A302153 | | |
| 87473 Lung NAT | 0.0 | 0.2 | Uterus Cancer GENPAK | 1.4 | 2.5 |
| (OD04451-02) | | | 064011 | | |
| 87474 Kidney Cancer | 100.0 | 100.0 | genomic DNA control | 0.0 | 0.8 |
| (OD04622-01) | | | | | |
| 87475 Kidney NAT | 1.0 | 5.8 | | | |
| (OD04622-03) | | | | | |

These results indicate that NOVNEUR is expressed in several normal cell and tissue lines, and in several cancer cell lines, including central nervous system (CNS) cancer (glio/astro and neuro; metastasis), lung cancer (non small cell), breast cancer, colon cancer and ovarian cancer (see Table 4). In addition, in comparison to surgical normal adjacent tissue, the clone is expressed in kidney cancer (clear cell type), prostate cancer, kidney cancer and thyroid cancer, as well as in lung cancer and kidney cancer (see Table 3). These results suggest that NOVNEUR may be used as a specific diagnostic probe for several types of cancer, and that the gene product may serve as a target for an antibody or for a small molecule drug in the treatment of several cancers.

C) RTQ PCR of NOVINTRA C Nucleic Acid

RTQ PCR of the NOVINTRA C nucleic acid sequence of the invention (SEQ ID NO: 11; see also FIG. 15A) was carried out using the primer-probe set Ag903 designated in Table 6, below. The expression of this gene was examined on tissues and cells from a variety of sources in Panel 1 (shown in Table 7), in cancer and tumor surgical samples of Panel 2 (shown in Table 8), and in a variety of tissues and cells related to inflammatory conditions in Panel 4 (described below; shown in Table 9, below).

TABLE 6
Primer-probe set Ag903 for the NOVINTRA C nucleic acid sequence.

| | | | Start |
|---|---|---|---|
| Primers | Sequences | Length | Position |
| Forward | 5′-TGAAGCTTCAGCTGCAGTGT-3′ | 20 | 160 |
| Probe | FAM-5′-CCGACTTTAGCACACATCAGGCAGAG-3′-TAMRA | 26 | 190 |
| Reverse | 5′-GGGCCTGAATGGACTCAAT-3′ | 19 | 216 |

TABLE 7
RTQ PCR results for the NOVINTRA C nucleic acid sequence on Panel 1.

| | Rel. | | Rel. |
|---|---|---|---|
| | Expr., | | Expr., |
| Cell source | % | Cell source | % |
| Liver adenocarcinoma | 0.0 | Renal ca. 786-0 | 0.0 |
| Heart (fetal) | 0.0 | Renal ca. A498 | 2.8 |
| Pancreas | 0.0 | Renal ca. RXF 393 | 0.0 |
| Pancreatic ca. CAPAN 2 | 0.0 | Renal ca. ACHN | 0.0 |
| Adrenal gland | 0.0 | Renal ca. UO-31 | 0.0 |
| Thyroid | 16.7 | Renal ca. TK-10 | 2.5 |
| Salivary gland | 18.6 | Liver | 0.0 |
| Pituitary gland | 0.0 | Liver (fetal) | 0.0 |
| Brain (fetal) | 0.0 | Liver ca. (hepatoblast) | 0.0 |
| | | HepG2 | |
| Brain (whole) | 0.0 | Lung | 0.0 |
| Brain (amygdala) | 1.5 | Lung (fetal) | 2.5 |
| Brain (cerebellum) | 0.0 | Lung ca. (small cell) LX-1 | 0.0 |
| Brain (hippocampus) | 0.0 | Lung ca. (small cell) NCI- | 0.0 |
| | | H69 | |
| Brain (thalamus) | 0.0 | Lung ca. (s.cell var.) SHP- | 0.0 |
| | | 77 | |
| Cerebral Cortex | 0.0 | Lung ca. (large cell) NCI- | 0.0 |
| | | H460 | |
| Spinal cord | 74.7 | Lung ca. (non-sm. cell) A549 | 0.0 |
| CNS ca. (glio/astro) U87-MG | 56.6 | Lung ca. (non-s.cell) NCI- | 0.0 |
| | | H23 | |
| CNS ca. (glio/astro) U-118- | 2.0 | Lung ca (non-s.cell) HOP-62 | 0.0 |
| MG | | | |
| dNS ca. (astro) SW1783 | 0.0 | Lung ca. (non-s.cl) NCI-H522 | 0.0 |
| CNS ca.* (neuro; met) SK-N- | 0.0 | Lung ca. (squam.) SW 900 | 0.0 |
| AS | | | |
| CNS ca. (astro) SF-539 | 0.0 | Lung ca. (squam.) NCI-H596 | 0.0 |
| CNS ca. (astro) SNB-75 | 0.0 | Mammary gland | 88.3 |
| CNS ca. (glio) SNB-19 | 0.0 | Breast ca.* (pl. effusion) | 0.0 |
| | | MCF-7 | |
| CNS ca. (glio) U251 | 0.0 | Breast ca.* (pl.ef) MDA-MB- | 0.0 |
| | | 231 | |
| CNS ca. (glio) SF-295 | 0.0 | Breast ca.* (pl.effusion) T47D | 0.0 |
| Heart | 0.0 | Breast ca. BT-549 | 0.0 |
| Skeletal muscle | 0.0 | Breast ca. MDA-N | 0.0 |
| Bone marrow | 0.0 | Ovary | 0.0 |
| Thymus | 57.4 | Ovarian ca. OVCAR-3 | 0.0 |
| Spleen | 0.0 | Ovarian ca. OVCAR-4 | 0.0 |
| Lymph node | 9.5 | Ovarian ca. OVCAR-5 | 0.0 |
| Colorectal | 0.0 | Ovarian ca. OVCAR-8 | 0.0 |
| Stomach | 100.0 | Ovarian ca. IGROV-1 | 0.0 |
| Small intestine | 0.0 | Ovarian ca.* (ascites) SK- | 0.0 |
| | | OV-3 | |
| Colon ca. SW480 | 0.0 | Uterus | 0.0 |
| Colon ca.* (SW480 met) SW620 | 0.0 | Plancenta | 0.0 |
| Colon ca. HT29 | 0.0 | Prostate | 1.9 |
| Colon ca. HCT-116 | 0.0 | Prostate ca.* (bone met) PC-3 | 0.0 |
| Colon ca. CaCo-2 | 0.0 | Testis | 0.0 |
| 83219 CC Well to Mod Diff | 0.0 | Melanoma Hs688(A).T | 0.0 |
| (ODO3866) | | | |
| Colon ca. HCC-2998 | 0.0 | Melanoma* (met) Hs688(B).T | 0.0 |
| Gastric ca.* (liver met) | 0.0 | Melanoma UACC-62 | 0.0 |
| NCI-N87 | | | |
| Bladder | 0.0 | Melanoma M14 | 2.7 |
| Trachea | 48.3 | Melanoma LOX IMVI | 2.7 |
| Kidney | 0.0 | Melanoma* (met) SK-MEL-5 | 0.0 |
| Kidney (fetal) | 0.0 | Adipose | 0.0 |
| Liver adenocarcinoma | 0.0 | Renal ca. 786-0 | 0.0 |

TABLE 8
RTQ PCR results for the NOVINTRA C nucleic acid sequence on Panel 2.

| | Rel. | | Rel. |
|---|---|---|---|
| | Expr., | | Expr., |
| Cell source | % | Cell source | % |
| Normal Colon GENPAK 061003 | 0.2 | Kidney NAT Clontech 8120608 | 0.0 |
| 83219 CC Well to Mod Diff | 0.0 | Kidney Cancer Clontech | 0.0 |
| (ODO3866) | | 8120613 | |
| 83220 CC NAT (ODO3866) | 0.3 | Kidney NAT Clontech 8120614 | 0.0 |
| 83221 CC Gr.2 rectosigmoid | 0.0 | Kidney Cancer Clontech | 0.0 |
| (ODO3868) | | 9010320 | |
| 83222 CC NAT (ODO3868) | 0.0 | Kidney NAT Clontech 9010321 | 0.1 |
| 83235 CC Mod Diff (ODO3920) | 0.1 | Normal Uterus GENPAK 061018 | 0.0 |
| 83236 CC NAT (ODO3920) | 0.0 | Uterus Cancer GENPAK 064011 | 0.0 |
| 83237 CC Gr.2 ascend colon | 0.2 | Normal Thyroid Clontech A+ | 1.4 |
| (ODO3921) | | 6570-1 | |
| 83238 CC NAT (ODO3921) | 0.2 | Thyroid Cancer GENPAK 064010 | 0.0 |
| 83241 CC from Partial | 0.1 | Thyroid Cancer INVITROGEN | 0.0 |
| Hepatectomy (ODO4309) | | A302152 | |
| 83242 Liver NAT (ODO4309) | 0.0 | Thyroid NAT INVITROGEN | 0.0 |
| | | A302153 | |
| 87472 Colon mets to lung | 0.0 | Normal Breast GENPAK 061019 | 0.0 |
| (OD04451-01) | | | |
| 87473 Lung NAT (OD04451-02) | 0.0 | 84877 Breast Cancer | 0.0 |
| | | (OD04566) | |
| Normal Prostate Clontech A+ | 0.8 | 85975 Breast Cancer | 0.0 |
| 6546-1 | | (OD04590-01) | |
| 84140 Prostate Cancer | 0.0 | 85976 Breast Cancer Mets | 0.0 |
| (OD04410) | | (OD04590-03) | |
| 84141 Prostate NAT (OD04410) | 0.0 | 87070 Breast Cancer | 0.3 |
| | | Metastasis (OD04655-05) | |
| 87073 Prostate Cancer | 0.0 | GENPAK Breast Cancer 064006 | 0.0 |
| (OD04720-01) | | | |
| 87074 Prostate NAT (OD04720- | 0.0 | Breast Cancer Clontech | 0.0 |
| 02) | | 9100266 | |
| Normal Lung GENPAK 061010 | 0.3 | Breast NAT Clontech 9100265 | 0.0 |
| 83239 Lung Met to Muscle | 0.0 | Breast Cancer INVITROGEN | 0.0 |
| (ODO4286) | | A209073 | |
| 83240 Muscle NAT (ODO4286) | 0.0 | Breast NAT INVITROGEN | 0.0 |
| | | A2090734 | |
| 84136 Lung Malignant Cancer | 0.0 | Normal Liver GENPAK 061009 | 0.0 |
| (OD03126) | | | |
| 84137 Lung NAT (OD03126) | 0.0 | Liver Cancer GENPAK 064003 | 0.1 |
| 84871 Lung Cancer (OD04404) | 100.0 | Liver Cancer Research | 0.0 |
| | | Genetics RNA 1025 | |
| 84872 Lung NAT (OD04404) | 0.0 | Liver Cancer Research | 0.0 |
| | | Genetics RNA 1026 | |
| 84875 Lung Cancer (OD04565) | 0.7 | Paired Liver Cancer Tissue | 0.0 |
| | | Research Genetics RNA 6004-T | |
| 85950 Lung Cancer (OD04237- | 0.0 | Paired Liver Tissue Research | 0.1 |
| 01) | | Genetics RNA 6004-N | |
| 85970 Lung NAT (OD04237-02) | 0.0 | Paired Liver Cancer Tissue | 0.0 |
| | | Research Genetics RNA 6005-T | |
| 83255 Ocular Mel Met to | 0.0 | Paired Liver Tissue Research | 0.0 |
| Liver (ODO4310) | | Genetics RNA 6005-N | |
| 83256 Liver NAT (OD04310) | 0.0 | Normal Bladder GENPAK 061001 | 0.2 |
| 84139 Melanoma Mets to Lung | 0.0 | Bladder Cancer Research | 0.3 |
| (OD04321) | | Genetics RNA 1023 | |
| 84138 Lung NAT (ODO4321) | 0.0 | Bladder Cancer INVITROGEN | 0.6 |
| | | A302173 | |
| Normal Kidney GENPAK 061008 | 0.1 | 87071 Bladder Cancer | 0.3 |
| | | (OD04718-01) | |
| 83786 Kidney Ca, Nuclear | 0.0 | 87072 Bladder Normal | 0.0 |
| grade 2 (OD04338) | | Adjacent (OD04718-03) | |
| 83787 Kidney NAT (OD04338) | 0.1 | Normal Ovary Res. Gen. | 0.0 |
| 83788 Kidney Ca Nuclear | 0.0 | Ovarian Cancer GENPAK 064008 | 0.0 |
| grade 1/2 (OD04339) | | | |
| 83789 Kidney NAT (OD04339) | 0.1 | 87492 Ovary Cancer (OD04768- | 0.0 |
| | | 07) | |
| 83790 Kidney Ca, Clear cell | 0.0 | 87493 Ovary NAT (OD04768- | 0.0 |
| type (OD04340) | | 08) | |
| 83791 Kidney NAT (OD04340) | 0.1 | Normal Stomach GENPAK 061017 | 0.0 |
| 83792 Kidney Ca, Nuclear | 0.1 | NAT Stomach Clontech 9060359 | 0.0 |
| grade 3 (OD04348) | | | |
| 83793 Kidney NAT (OD04348) | 0.0 | Gastric Cancer Clontech | 0.0 |
| | | 9060395 | |
| 87474 Kidney Cancer | 0.0 | NAT Stomach Clontech 9060394 | 0.0 |
| (OD04622-01) | | | |
| 87475 Kidney NAT (OD04622- | 0.0 | Gastric Cancer Clontech | 0.0 |
| 03) | | 9060397 | |
| 85973 Kidney Cancer | 0.0 | NAT Stomach Clontech 9060396 | 0.0 |
| (OD04450-01) | | | |
| 85974 Kidney NAT (OD04450- | 0.0 | Gastric Cancer GENPAK 064005 | 0.0 |
| 03) | | | |
| Kidney Cancer Clontech | 0.0 | | |
| 8120607 | | | |
| Normal Colon GENPAK 061003 | 0.2 | | |

The RTQ PCR results in Table 8 indicate that in one lung cancer sample, the tumor overexpresses NOVINTRA C, compared with the NAT, the normal lung, or any tissue. This result suggests that a monoclonal antibody directed against the TRA C protein could be used as a diagnostic tool for a subset of lung cancer, and as a tic tool to treat lung cancer in a set of patients including those having this type of lung cancer.

Panel 4 was prepared in a 96 well plate (2 control wells, 94 test samples), containing RNA or cDNA isolated from various human cell lines or tissues related to inflammatory conditions. Total RNA from control normal tissues: colon, and lung were purchased from Stratagene (La Jolla, Calif.); thymus and kidney total RNA was obtained from Clontech (Palo Alto, Calif.). Total RNA from liver tissue from Cirrhosis patients and kidney from Lupus patients were obtained from Biochain. Intestinal tissue for RNA preparation from Crohns disease and ulcerative colitis patients was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. For endothelial cells we sometimes starved the cells for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA or PWM at approximately 5 μg/ml. Samples were taken at 24, 48 and 72 hours for RNA preparation. MLR samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

To prepare monocytes, macrophages and dendritic cells, monocytes were isolated from mononuclear cells using CD14 Miltenyi Beads, +ve VS selection columns and a Vario Magnet as per the manufacturer's instructions. Monocytes were differentiated into dendritic cells by culture in DMEM 5% FCS (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with LPS at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet as per the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD4RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercatoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM nonessential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. We also obtained a keratinocyte line CCD106 and an airway epithelial tumor line NCI-H292 from the ATCC. Both were cultured in DMEM 5% FCS (Hyclone), 100 μM nonessential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, we prepared RNA by lysing approximately 10⁷ cells/ml using Trizol (Gibco BRL). Briefly, 1/10 volume of Bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol.

To remove any genomic DNA contamination from the resulting total RNA preparations, they were treated with DNase (2 μl; 10 u/μl; Qiagen, cat.# 79254) in the presence of 1×DNase buffer from Promega for 30 min at 37 ° C. RNA was extracted by addition of equal volumes of acid phenol: chloroform (Ambion, cat.# 9720), followed by precipitation from the aqueous phase with 0.3 M sodium acetate (Fluka, cat.# 71196) and two volumes of ethanol. The precipitate was recovered by spinning as above, washed once with 70% ethanol and resuspended in 50 μl DEPC treated water.

RNA was quantitated fluorometrically (Tecan SpectraFluor Plus) using a RNA specific dye, Ribogreen (Molecular Probes, Eugene OR; Catalog number R-11491) according to the manufacturer's directions. The quality of the RNA was determined by running the RNA either on agarose-formaldehyde gels or RNA chips (Agilent 5064-8229) from Agilent Technology (2100 Bioanalyser).

The RNA samples for each cell or tissue were normalized according to RNA input by RNA quantification using Ribogreen (as described above) using a standard curve covering the concentration range of 1 ng/ml through 50 ng/ml RNA.

Absence of genomic DNA contamination in every RNA sample was confirmed by monitoring the expression of human polypeptide chain elongation factor-1 alpha (GenBank Accession Number: E02629) and human ADP-ribosylation factor 1 (ARF1) mRNA (GenBank Accession Number: M36340) by TAQMAN®, without performing a reverse transcription step prior to the PCR cycles (minus RT-TAQMAN® assay). Ten ng of RNA (total or polyA+) were used in a 25 ul TAQMAN® reaction using probe and primer sets specific for intronless segments of human polypeptide chain elongation factor-1 alpha and human ADP-ribosylation factor 1 (ARF1) mRNA. Probe and primers sets were designed for each assay according to a proprietary software package. Reactions were carried out using the TAQMAN® universal PCR Master Mix (Applied Biosystems, Foster City, Calif., USA; cat # 4304447) according to the manufacturer's protocol. Reactions were performed using 96 well optical plates and caps (Applied Biosystems, cat # 403012) on an ABI Prism 7700® Sequence Detection System (Applied Biosystems) using the following parameters: 10 min at 95° C.; 15 sec at 95° C./1 min at 60° C. (40 cycles). Results were recorded as CT values (cycle at which a given sample crosses a threshold level of fluorescence) using a log scale. Any sample showing a CT value lower than 35 for any of the two tested genes were treated again with DNAse 1 following the protocol described above.

RNA (2-10 μg total or polyA+) was converted to cDNA using Superscript II(Life Tech; cat# 18064-147 ) and random hexamers. Reactions were performed in a volume of 20 μl and incubated for 60 mins at 42° C. to generate the single stranded cDNA (sscDNA).

sscDNA was then diluted in DEPC-water to a final concentration of 0.2 ng/μl (assuming a 1:1 RNA to cDNA conversion ratio). Five μl of sscDNA was transferred to a separate plate for the TAQMAN® reaction using probe and primer sets specific for human polypeptide chain elongation factor-1 alpha and human ADP-ribosylation factor 1 (ARF1) mRNA. TAQMAN® reactions were performed following the minus RT-TAQMAN® assay protocol described previously. Results were recorded as CT values, with the difference in RNA concentration between a given sample and the sample with the lowest CT value being represented as 2 to the power of delta CT (2^{ΔCT}). The percent relative expression is then obtained by taking the reciprocal of this RNA difference and multiplying by 100. The median CT values obtained for two housekeeping genes: human polypeptide chain elongation factor-1 alpha (hEF-1α) and human ADP-ribosylation factor 1 (hARF1) were used to normalize sscDNA samples within each panel. The concentrations of the sscDNA samples were adjusted so as to be within the median CT value, +/−one CT unit for these two housekeeping genes. After every round of sscDNA concentration adjustment, the relative gene expression for hEF-1α and hARF 1 sscDNA was measured by TAQMAN® as described previously.

The results obtained for NOVINTRA C nucleic acid sequence on Panel 4 using the primer-probe set Ag903 are shown in Table 9, below.

TABLE 9
RTQ PCR results for the NOVINTRA C nucleic acid sequence on Panel 4.

| | | | | | Rel. |
|---|---|---|---|---|---|
| | Rel. | Rel. | | Rel. | Expr |
| | Expr., | Expr., | | Expr., | ., |
| | %, | %, | | %, | %, |
| | 2tm515 | 2tm1027 | | 2tm515 | 2tml |
| Tissue Source | f | f | Tissue Source | f | 027f |
| 93768_Secondary Th1_anti- | 1.1 | 0.0 | 93100_HUVEC | 0.0 | 0.0 |
| CD28/anti-CD3 | | | (Endothelial)_IL-lb | | |
| 93769_Secondary Th2_anti- | 1.3 | 0.0 | 93779_HUVEC | 0.2 | 0.0 |
| CD28/anti-CD3 | | | (Endothelial)_IFN | | |
| | | | gamma | | |
| 93770_Secondary Tr1_anti- | 1.3 | 0.0 | 93102_HUVEC | 0.0 | 0.0 |
| CD28/anti-CD3 | | | (Endothelial)_TNF | | |
| | | | alpha + IFN gamma | | |
| 93573_Secondary Th1_resting | 5.3 | 0.6 | 93101_HUVEC | 0.7 | 0.0 |
| day 4-6 in IL-2 | | | (Endothelial)_TNF | | |
| | | | alpha + IL4 | | |
| 93572_secondary Th2_resting | 0.0 | 1.0 | 93781_HUVEC | 0.0 | 0.0 |
| day 4-6 in IL-2 | | | (Endothelial)_IL-11 | | |
| 93571_secondary Tr1 _resting | 1.6 | 4.3 | 93583_Lung | 0.0 | 0.0 |
| day 4-6 in IL-2 | | | Microvascular | | |
| | | | Endothelial | | |
| | | | Cells_none | | |
| 93568_primary Th1_anti- | 1.8 | 1.7 | 93584_Lung | 0.0 | 0.0 |
| CD28/anti-CD3 | | | Microvascular | | |
| | | | Endothelial | | |
| | | | Cells_TNFa (4 | | |
| | | | ng/ml) and IL1b (1 | | |
| | | | ng/ml) | | |
| 93569_primary Th2_anti- | 2.2 | 1.1 | 92662_Microvascular | 0.0 | 0.0 |
| CD28/anti-CD3 | | | Dermal | | |
| | | | endothelium_none | | |
| 93570_primary Tr1_anti- | 3.5 | 6.0 | 92663_Microsvasular | 0.0 | 0.0 |
| CD28/anti-CD3 | | | Dermal | | |
| | | | endothelium_TNFa (4 | | |
| | | | ng/ml) and IL1b (1 | | |
| | | | ng/ml) | | |
| 93565_primary Th1_resting | 16.7 | 18.1 | 93773_Bronchial | 0.6 | 0.0 |
| dy 4-6 in IL-2 | | | epithelium_TNFa (4 | | |
| | | | ng/ml) and IL1b (1 | | |
| | | | ng/ml) ** | | |
| 93566_primary Th2_resting | 15.5 | 12.8 | 93347_Small Airway | 0.6 | 0.0 |
| dy 4-6 in IL-2 | | | Epithelium_none | | |
| 93567_primary Tr1_resting | 12.5 | 9.3 | 93348_Small Airway | 100.0 | 100.0 |
| dy 4-6 in IL-2 | | | Epithelium_TNFa (4 | | |
| | | | ng/ml) and IL1b (1 | | |
| | | | ng/ml) | | |
| 93351_CD45RA CD4 | 0.0 | 0.6 | 92668_Coronery | 0.0 | 0.0 |
| lymphocyte_anti-CD28/anti- | | | Artery SMC_resting | | |
| CD3 | | | | | |
| 93352_CD45RO CD4 | 1.9 | 0.0 | 92669_Coronery | 0.0 | 0.0 |
| lymphocyte anti-CD28/anti- | | | Artery SMC_TNFa (4 | | |
| CD3 | | | ng/ml) and IL1b (1 | | |
| | | | ng/ml) | | |
| 93251_CD8 Lymphocytes_anti- | 1.3 | 1.1 | 93107_astrocytes_re | 0.0 | 0.0 |
| CD28/anti-CD3 | | | sting | | |
| 93353_chronic CD8 | 1.2 | 0.0 | 93108_astrocytes_TN | 0.0 | 0.0 |
| Lymphocytes 2ry_resting dy | | | Fa (4 ng/ml) and | | |
| 4-6 in IL-2 | | | IL1b (1 ng/ml) | | |
| 93574_chronic CD8 | 1.8 | 0.8 | 92666_KU-812 | 0.0 | 0.0 |
| Lymphocytes 2ry_activated | | | (Basophil)_resting | | |
| CD3/CD28 | | | | | |
| 93354_CD4_none | 4.5 | 3.6 | 92667_KU-812 | 0.0 | 0.0 |
| | | | (Basophil)_PMA/iono | | |
| | | | ycin | | |
| 93252_Secondary | 5.9 | 1.2 | 93579_CCD1106 | 0.0 | 0.0 |
| Th1/Th2/Tr1_anti-CD55 CH11 | | | (Keratinocytes)_non | | |
| | | | e | | |
| 93103_LAK cells_resting | 0.9 | 0.6 | 93580_CCD11066 | 0.6 | 0.0 |
| | | | (Keratinocytes)_TNF | | |
| | | | a and IFNg ** | | |
| 93788_LAK cells_IL-2 | 0.0 | 0.0 | 93791_Liver | 5.8 | 4.5 |
| | | | Cirrhosis | | |
| 93787 LAK cells_IL-2 + IL-12 | 1.5 | 0.5 | 93792_Lupus Kidney | 0.0 | 0.0 |
| 93789_LAK cells_IL-2 + IFN | 4.6 | 3.1 | 93577_NCI-H292 | 0.6 | 0.0 |
| gamma | | | | | |
| 93790_LAK cells_IL-2 + IL-18 | 1.2 | 1.7 | 93358_NCI-H292_IL-4 | 0.0 | 0.0 |
| 93104_LAK | 0.0 | 0.0 | 93360_NCI-H292_IL-9 | 0.0 | 0.0 |
| cells_PMA/ionomycin and IL- | | | | | |
| 18 | | | | | |
| 93578_NK Cells IL-2_resting | 0.6 | 0.0 | 93359_NCI-H292_IL- | 0.0 | 0.6 |
| | | | 13 | | |
| 93109_Mixed Lymphocyte | 0.0 | 0.0 | 93357_NCI-H292_IFN | 0.0 | 1.1 |
| Reaction_Two Way MLR | | | gamma | | |
| 93110_Mixed Lymphocyte | 0.5 | 0.0 | 93777_HPAEC_- | 0.0 | 0.0 |
| Reaction_Two Way MLR | | | | | |
| 93111_Mixed Lymphocyte | 0.0 | 0.0 | 93778_HPAEC_IL-1 | 0.0 | 0.6 |
| Reaction_Two Way MLR | | | beta/TNA alpha | | |
| 93112_Mononuclear Cells | 0.6 | 1.1 | 93254_Normal Human | 0.0 | 0.0 |
| (PBMCs)_resting | | | Lung | | |
| | | | Fibroblast_none | | |
| 93113_Mononuclear Cells | 1.2 | 0.0 | 93253_Normal Human | 0.0 | 0.0 |
| (PBMCs)__PWM | | | Lung | | |
| | | | Fibroblast_TNFa (4 | | |
| | | | ng/ml) and IL-1b (1 | | |
| | | | ng/ml) | | |
| 93114_Mononuclear Cells | 6.9 | 1.0 | 93257_Normal Human | 0.0 | 0.0 |
| (PBMCs)_PHA-L | | | Lung Fibroblast IL- | | |
| | | | 4 | | |
| 93249_Ramos (B cell)_none | 0.0 | 0.7 | 93256_Normal Human | 0.0 | 0.0 |
| | | | Lung Fibroblast_IL- | | |
| | | | 9 | | |
| 93250_Ramos (B | 0.0 | 0.0 | 93255_Normal Human | 0.0 | 0.0 |
| cell)_ionomycin | | | Lung Fibroblast_IL- | | |
| | | | 13 | | |
| 93349_B lymphocytes_PWM | 1.5 | 3.3 | 93258_Normal Human | 0.0 | 0.0 |
| | | | Lung Fibroblast_IFN | | |
| | | | gamma | | |
| 93350_B lymphocytes_CD40L | 1.3 | 0.7 | 93106_Dermal | 0.0 | 0.0 |
| and IL-4 | | | Fibroblasts | | |
| | | | CCD1070_resting | | |
| 92665_EOL-1 | 0.0 | 0.0 | 93361_Dermal | 0.6 | 0.5 |
| (Eosinophil)_dbcAMP | | | Fibroblasts | | |
| differentiated | | | CCD1070_TNF alpha 4 | | |
| | | | ng/ml | | |
| 93248_EOL-1 | 0.0 | 0.0 | 93105_Dermal | 0.0 | 0.0 |
| (Eosinophil)_dbcAMP/PMAiono | | | Fibroblasts | | |
| mycin | | | CCD1070_IL-1 beta 1 | | |
| | | | ng/ml | | |
| 93356_Dendritic Cells_none | 0.0 | 0.0 | 93772_dermal | 0.0 | 0.0 |
| | | | fibroblast_IFN | | |
| | | | gamma | | |
| 93355_Dendritic Cells_LPS | 0.0 | 0.0 | 93771_dermal | 0.0 | 0.7 |
| 100 ng/ml | | | fibroblast_IL-4 | | |
| 93775_Dendritic Cells_anti- | 0.0 | 0.0 | 93259_IBD Colitis | 10.4 | 0.0 |
| CD40 | | | 1** | | |
| 93774_Monocytes_resting | 0.0 | 0.0 | 93260_IBD Colitis 2 | 0.0 | 0.0 |
| 93776_Monocytes_LPS 50 | 2.9 | 0.8 | 93261_IBD Crohns | 0.0 | 0.0 |
| ng/ml | | | | | |
| 93581_Macrophages_resting | 0.0 | 0.0 | 735010_Colon_normal | 1.0 | 0.0 |
| 93582_Macrophages_LPS 100 | 0.0 | 0.0 | 735019_Lung_none | 0.0 | 0.0 |
| ng/ml | | | | | |
| 93098_HUVEC | 0.7 | 0.3 | 64028-1_Thymus_none | 0.0 | 0.0 |
| (Endothelial)_none | | | | | |
| 93099_HUVEC | 0.0 | 0.0 | 64030-1_Kidney_none | 11.5 | 7.2 |
| (Endothelial)_starved | | | | | |

The results obtained for Panel 4 (Table C4) indicate a dramatic unexpected over-expression (greater than 100 fold) of NOVINTRA C by small airway epithelium treated with TNF alpha (4 ng/mL) and IL-1 beta (1 ng/mL) relative to the untreated cells. The expression pattern is highly specific.

The results of these Real Time Quantitative PCR (TaqMan) tissue expression experiments suggest that NOVINTRA C (IL-1 Epsilon) is differentially expressed in TNF-alpha treated small airway epithelium. This previously unreported result indicates a possible role for NOVINTRA C (IL-1 Epsilon) in asthma, irritation in the lungs due to allergies and inflammatory conditions in diseases such as emphysema. Currently, no diagnostic test exists for the presence of IL-1 Epsilon (NOVINTRA C) in the lungs of asthmatic patients or those suffering from irritation of the airways due to allergies. Providing an accurate indicator of the presence and measurement of the amount of IL-1 Epsilon may assist in the diagnosis and treatment of asthmatic and allergy patients. The protein of invention presented here, NOVINTRA C (IL-1 Epsilon), could thus be used as a monoclonal antibody target in Enzyme Linked Immunosorbent Assays (ELISA) to provide a means of detection for IL-1 Epsilon. Using procedures similar to those described by Teran, et al., Clin Exp Allergy. April;27(4):396-405 (1997); Kelley et al., Am J Respir Crit Care Med. September;162(3 Pt 1):883-90 (2000), and Hasday et al., Am J Respir Crit Care Med. April;161(4 Pt 1):1229-36 (2000); bronchoalveolar or nasal lavage (BAL, NL) fluid from allergy and asthmatic patients may be obtained and assayed in ELISA experiments to quantify the relative amount of IL-1 Epsilon. This diagnostic tool should allow for more efficient identification and alleviation of symptoms of inflammation in asthma and allergy patients.

In addition, the expression profile of NOVINTRA C (IL-1 Epsilon) has demonstrated that it has disease association with asthma, allergy and emphysema. It may play a potential role in the development of these diseases. Therefore it has potential usefulness as a therapeutic target, for example, as a target for an IL-1 Epsilon-specific monoclonal antibody, other protein therapeutic or small molecule therapeutic.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising an amino acid sequence selected from the group consisting of:
(a) a mature form of an amino acid sequence selected from the group consisting of SEQ ID NOs: 2, 4, 6, 8, and 10;
(b) a variant of a mature form an amino acid sequence selected from the group consisting of SEQ ID NOs: 2, 4, 6, 8, and 10, wherein one or more amino acid residues in said variant differs from the amino acid sequence of said mature form, provided that said variant differs in no more than 10% of the amino acid residues from the amino acid sequence of said mature form;
(c) an amino acid sequence comprising a sequence selected from the group consisting of SEQ ID NOs: 2, 4, 6, 8, and 10; and
(d) a variant of an amino acid sequence comprising a sequence selected from the group consisting of SEQ ID NOs: 2, 4, 6, 8 and 10, wherein one or more amino acid residues in said variant differs from the amino acid sequence of said mature form, provided that said variant differs in no more than 10% of amino acid residues from said amino acid sequence.

2. The polypeptide of claim 1, wherein said polypeptide comprises an amino acid sequence of a naturally-occurring allelic variant of an amino acid sequence selected from the group consisting of SEQ ID NOs: 2, 4, 6, 8, and 10.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOs: 1, 3, 5, 7, and 9.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOs: 2, 4, 6, 8 and 10;
(b) a variant of a mature form of an amino acid sequence selected from the group consisting of SEQ ID NOs: 2, 4, 6, 8, and 10, wherein one or more amino acid residues in said variant differs from the amino acid sequence of said mature form, provided that said variant differs in no more than 10% of the amino acid residues from the amino acid sequence of said mature form;
(c) an amino acid sequence comprising a sequence selected from the group consisting of SEQ ID NOs: 2, 4, 6, 8, and 10;
(d) a variant of an amino acid sequence selected from the group consisting of SEQ ID NOs: 2, 4, 6, 8, and 10, wherein one or more amino acid residues in said variant differs from the amino acid sequence of said mature form, provided that said variant differs in no more than 10% of amino acid residues from said amino acid sequence;
(e) a nucleic acid fragment encoding at least a portion of a NOVX polypeptide comprising an amino acid sequence comprising a sequence selected from the group consisting of SEQ ID NOs: 2, 4, 6, 8, and 10, or a variant of said polypeptide, wherein one or more amino acid residues in said variant differs from the amino acid sequence of said mature form, provided that said variant differs in no more than 10% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOs: 1, 3, 5, 7, and 9.

9. The nucleic acid molecule of claim 5, wherein said nucleic acid molecule comprises a nucleotide sequence selected from the group consisting of
(a) a nucleotide sequence selected from the group consisting of SEQ ID NOs: 1, 3, 5, 7, and 9;
(b) a nucleotide sequence differing by one or more nucleotides from a nucleotide sequence selected from the group consisting of SEQ ID NOs: 1, 3, 5, 7, and 9, provided that no more than 10% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence selected from the group consisting of SEQ ID NOs: 1, 3, 5, 7, and 9, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 10% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX protein-associated disorder, said method comprising administering to a subject in which such treatment or prevention is desired the polypeptide of claim 1 in an amount sufficient to treat or prevent said NOVX protein-associated disorder in said subject.

24. The method of claim 23, wherein said subject is a human.

25. A method of treating or preventing a NOVX protein-associated disorder, said method comprising administering to a subject in which such treatment or prevention is desired the nucleic acid of claim 5 in an amount sufficient to treat or prevent said NOVX protein-associated disorder in said subject.

26. The method of claim 25, wherein said subject is a human.

27. A method of treating or preventing a NOVX protein-associated disorder, said method comprising administering to a subject in which such treatment or prevention is desired the antibody of claim 15 in an amount sufficient to treat or prevent said NOVX protein-associated disorder in said subject.

28. The method of claim 15, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

36. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

37. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence selected from the group consisting of SEQ ID NOs: 2, 4, 6, 8, 10, or 12, or a biologically active fragment thereof.

38. A method of treating a pathological state in a mammal, the method comprising administering to the mammal the antibody of claim 15 in an amount sufficient to alleviate said pathological state.

39. A method of treating a NOVX protein-related disorder in a mammal, the method comprising administering to the mammal at least one agent which modulates the expression or activity of a NOVX protein.

40. The method of claim 39, wherein said NOVX protein is a novel human transmembrane protein (NOVTRAN), and wherein said disorder is a cell signaling disorder selected from the group consisting of cancer, an immune response disorder, a hematopoietic disorder, and a neurodegenerative disorder.

41. The method of claim 39, wherein said NOVX protein is a novel human neuromedin protein (NOVNEUR), and wherein said disorder is selected from the group consisting of an endocrine disorder, a muscle disorder, a neurologic disorder, central nervous system cancer, breast cancer, colon cancer, ovarian cancer, kidney cancer, prostate cancer, and thryorid cancer.

42. The method of claim 39, wherein said NOVX protein is a novel human gonadotropin protein (NOVGON), and wherein said disorder is selected from the group consisting of a reproductive development disorder, a metabolic function disorder, and melanoma.

43. The method of claim 39, wherein said NOVX protein is a novel human interleukin-1 receptor antagonist protein (NOVINTRA), and wherein said disorder is selected from the group consisting of a bone metabolism or structure disorder, and inflammatory response disorder, an immune regulation disorder, septic shock, stroke, diabetes, arthritis, and cancer.

44. The method of claim 39, wherein said agent is selected from the group consisting of an antibody which immunospecifically binds to a NOVX polypeptide, an antibody which immunospecifically binds to a nucleic acid sequence encoding a NOVX protein, and an antisense nucleic acid sequence complementary to a nucleic acid sequence encoding a NOVX protein.

45. A method for determining the presence of or predisposition to lung disease associated with altered levels of an interleukin-1 epsilon (IL-1 epsilon) polypeptide in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

46. The method of claim 45, wherein said lung disease is selected from the group consisting of lung cancer, asthma, emphysema, allergic lung irritation, and lung inflammation.

47. The method of claim 45, wherein said polypeptide comprises the amino acid sequence of SEQ ID NO: 12.

48. A method for determining the presence of or predisposition to a lung disease associated with altered levels of a nucleic acid molecule encoding human IL-1 epsilon in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

49. The method of claim 48, wherein said lung disease is selected from the group consisting of lung cancer, asthma, emphysema, allergic lung irritation, and lung inflammation.

50. The method of claim 49, wherein said nucleic acid molecule comprises the nucleic acid sequence of SEQ ID NO: 11.

51. A method of treating a lung disease in a mammal, the method comprising administering to the mammal at least one agent which modulates the expression or activity of a human IL-1 epsilon protein.

52. The method of claim 51, wherein said lung disease is selected from the group consisting of lung cancer, asthma, emphysema, allergic lung irritation, and lung inflammation.

53. The method of claim 52, wherein said IL-1 epsilon protein comprises the amino acid sequence of SEQ ID NO: 12.

54. The method of claim 53, wherein said agent is selected from the group consisting of an antibody which immunospecifically binds to said IL-1 epsilon protein, an antibody which immunospecifically binds to a nucleic acid sequence encoding said IL-1 epsilon protein, and an antisense nucleic acid sequence complementary to a nucleic acid sequence encoding said IL-1 epsilon protein.

55. The method of claim 54, wherein said nucleic acid sequence comprises the nucleic acid sequence of SEQ ID NO: 11.


SEQUENCE LISTING (100 sequences)

SEQ ID NO: 1 - DNA, 1047 bp, Homo sapiens
atgcagtggtcctgtctggcctgtaccctcctcagggtcctcccacatgtcctgtctctcctgagagaccctgtgcctgtgcccacagggaccaagctcttccactcctgtatcacctcaacgaacccatgcgcctccttcctggaggttgctgttgaagctgcaggcatcaccccctggactgtagggtctgagcacccgccctgtccatatccatccctgcatgcctctccgttcaccgactccttcaacagaccatcccctgctcctctcaacaggccccgctctgctggggaaccacggacagaggccttcccatccccaggcctgaaggccagagtaggtgggaccatcctcgccgaagccggcctcaattctcaaggccatgccgtggagccagtgccatctggaccctctgggtcaagcaaagggtgtgtgctaatcaaaggcaggccctcgaggatgccaaaggcccgcgaatgcccagtggaccgtgaaaaccttctgctgacaaaccctgcagtgccttctctgctccagctgctctccagctctccatgcatcaaggtggaaacagagcaggagcgcagtaatgcggaatttgacttgcaaagtcgggccgctcgggattacaattcaaggctgctgctgaaactcgggcagatcccagctgcaaagggcagttccttcctcgagctgcagaacgtgtctggaggggttggctcagcccgaggtcccaggaaccactgcaaggtgggggcgggccctcagagccctttcccagagctgggggctggtagcccccctttggctttggagaaggtcagtacccaacccattccccaggcccgactgcggaagggtgtggactggccccctgtgtctcctggtgaccagtgtccactgtgcactctcccaggccagccgaacctggcacacactgggtgttccctaaatagccatggagggtattgtggcatggagagctgtcgattccagaaacctcctggacatagggctgggagctcatctgcagaagctgcctga

SEQ ID NO: 2 - protein, 348 aa, Homo sapiens [sequence omitted]

SEQ ID NO: 3 - DNA, 646 bp, Homo sapiens
agcgcgcccgaacgaagccgcggcccgggcacagcatggcccgcggcgggagggcgctcggatgttcggcagcctcctgcacttcgccctgctcgctgccggcgtcgtcccgctcagctgggatctcccggagccccgcagccgagccagcaagatccgagtgcactcgcgaggcaagctctgggccatcggtcacttcatgggcaagaagagtctggagccttccagcccatccccattggggacagctccccacacctccctgagggaccagcgactgcagctgagtcatgatctgctcggaatcctcctgctaaagaaggctctgggcgtgagcctcagccgccccgcaccccaaatccagtacaggaggctgctggtacaaatactgcagaaatgacaccaataatggggcagacacaacagcgtggcttagattgtgcccacccagggaaggtgctgaatgggaccctgttgatggccccatctggatgtaaatcctgagctcaaatctctgttactccattactgtgatttctggctgggtcaccagaaatatcgctgatgcagacacagattatgttcctgctgtatttcctgcttccctgttgaattggtgaataaaaccttgctctatacatacaaa

SEQ ID NO: 4 - protein, 112 aa, Homo sapiens
MFGSLLHFALLAAGVVPLSWDLPEPRSRASKIRVHSRGKLWAIGHFMGKKSLEPSSPSPLGTAPHTSLRDQRLQLSHDLLGILLLKKALGVSLSRPAPQIQYRRLLVQILQK

SEQ ID NO: 5 - DNA, 693 bp, Homo sapiens
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagctgaggttgatgccaggggaagctgctgtggcactgggcttctggtgtcagcgtaggagacagggatctaggacaacagggaccaggtggcgacatgcagctgtaagagacaaggtgagtctcctgaaggcagtagatggttggaatgggctgcttggggacccagcgagctcccagggcctttctgcttcttcctgtacccctgtatttcccttggctttccaaattgactcagcttctggtaaagttggaaacttttccagcaaacagaccttcatcttctccagtgcagagattacattaggaggaacatga

SEQ ID NO: 6 - protein, 230 aa, Homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTELRLMPGEAAVALGFWCQRRRQGSRTTGTRWRHAAVRDKVSLLKAVDGWNGLLGDPASSQGLSASSCTPVFPLAFQIDSASGKVGNFSSKQTFIFSSAEITLGGT

SEQ ID NO: 7 - DNA, 483 bp, Homo sapiens
cactgtcatactgtttcagaattaaatatgcagaccagaaggctctatacacaagagatggccagctgctggtgggagatcctgttgcagacaactgctgtgcagagaagatctgcatacttcctaacagaggcttggcccgcaccaaggtccccattttcctggggatccagggagggagccgctgcctggcatgtgtggagacagaagaggggccttccctacagctggagccatccaccttgcccccacaggatgtgaacattgaggaactgtacaaaggtggtgaagaggccacacgcttcaccttcttccagagcagctcaggctccgccttcaggcttgaggctgctgcctggcctggctggttcctgtgtggcccggcagagccccagcagccagtacagctcaccaaggagagtgagccctcagcccgtaccaagttttactttgaacagagctggtagggagacaggaaactgc

SEQ ID NO: 8 - protein, 154 aa, Homo sapiens
LSYCFRIKYADQKALYTRDGQLLVGDPVADNCCAEKICILPNRGLARTKVPIFLGIQGGSRCLACVETEEGPSLQLEPSTLPPQDVNIEELYKGGEEATRFTFFQSSSGSAFRLEAAAWPGWFLCGPAEPQQPVQLTKESEPSARTKFYFEQSW

SEQ ID NO: 9 - DNA, 520 bp, Homo sapiens
atgggcacacctggcctggccctgcatgcagactggacggtgagccaggacttctgcaggacacccaaatcctatgctattcgtgattctcgacagatggtgtgggtcctgagtggaaattctttaatagcagctcctcttagccgcagcattaagcctgtcactcttcatttaatagcctgtagagacacagaattcagtgacaaggaaaagggtaatatggtttacctgggaatcaagggaaaagatctctgtctcttctgtgcagaaattcagggcaagcctactttgcagcttaaggaaaaaaatatcatggacctgtatgtggagaagaaagcacagaagccctttctctttttccacaataaagaaggctccacttctgtctttcagtcagtctcttaccctggctggttcatagccacctccaccacatcaggacagcccatctttctcaccaaggagagaggcataactaataacactaacttctacttagattctgtggaataaatccagc

SEQ ID NO: 10 - protein, 170 aa, Homo sapiens
MGTPGLALHADWTVSQDFCRTPKSYAIRDSRQMVWVLSGNSLIAAPLSRSIKPVTLHLIACRDTEFSDKEKGNMVYLGIKGKDLCLFCAEIQGKPTLQLKEKNIMDLYVEKKAQKPFLFFHNKEGSTSVFQSVSYPGWFIATSTTSGQPIFLTKERGITNNTNFYLDSVE

SEQ ID NO: 11 - DNA, 391 bp, Homo sapiens
gatatcaatcatcgggtgtgggttcttcaggaccagacgctcatagcagtcccgaggaaggtgttcccagtcactattgccttaatctcatgccgacatgtggagacccttgagaaagacagagggaaccccatctacctgggcctgaatggactcaatctctgcctgatgtgtgctaaagtcggggaccagcccacactgcagctgaagcttcaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaag

SEQ ID NO: 12 - protein, 130 aa, Homo sapiens
DINHRVWVLQDQTLIAVPRKVFPVTIALISCRHVETLEKDRGNPIYLGLNGLNLCLMCAKVGDQPTLQLKLQEKDIMDLYNQPEPVKSFLFYHSQSGRNSTFESVAFPGWFIAVSSEGGCPLILTQELGK

SEQ ID NO: 13 - DNA, 23 bp, Homo sapiens
acaacgagaccaaacaggtgact

SEQ ID NO: 14 - DNA, 22 bp, Homo sapiens
tcaagctgcccaactgtgcccc

SEQ ID NO: 15 - DNA, 20 bp, Homo sapiens
ggccacgggataggtgtaga

SEQ ID NO: 16 - DNA, 18 bp, Homo sapiens
ttccagcccatccccatt

SEQ ID NO: 17 - DNA, 22 bp, Homo sapiens
ccccacacctccctgagggacc

SEQ ID NO: 18 - DNA, 23 bp, Homo sapiens
cagatcatgactcagctgcagtc

SEQ ID NO: 19 - DNA, 117 bp, Homo sapiens
acctcgggctgagccaacccctccagacacgttctgcagctcgaggaaggaactgccctttgcagctgggatctgcccgagtttcagcagcagccttgaattgtaatcccgagcggc

SEQ ID NO: 20 - DNA, 117 bp, Homo sapiens; identical to SEQ ID NO: 19

SEQ ID NO: 21 - DNA, 636 bp, Homo sapiens
gcgcgcccgaacgaagccgcggcccgggcacagcatggcccgcggcgggagggcgctcggatgttcggcagcctcctgcacttcgccctgctcgctgccggcgtcgtcccgctcagctgggatctcccggagccccgcagccgagccagcaagatccgagtgcactcgcgaggcaagctctgggccatcggtcacttcatgggcaagaagagtctggagccttccagcccatccccattggggacagctccccacacctccctgagggaccagcgactgcagctgagtcatgatctgctcggaatcctcctgctaaagaaggctctgggcgtgagcctcagccgccccgcaccccaaatccagtacaggaggctgctggtacaaatactgcagaaatgacaccaataatggggcagacacaacagcgtggcttagattgtgcccacccagggaaggtgctgaatgggaccctgttgatggccccatctggatgtaaatcctgagctcaaatctctgttactccattactgtgatttctggctgggtcaccagaaatatcgctgatgcagacacagattatgttcctgctgtatttcctgcttccctgttgaattggtgaataaaaccttgctctat

SEQ ID NO: 22 - DNA, 629 bp, Homo sapiens
gcgcgcccgaacgaagccgcggcccgggcacagcatggcccgcggcggggggcgctcggatgttcggcagcctcctgccttcgccctgctcgctgccggcgtcgcccgctcagctgggatctcccggagccccgcagccgagccagcaagatccgagtgcactcgcgaggcaactctgggccacggtcacttcatgggcaagaagagtctggagccttccagcccatcccattggggacagctccccacacctccctgagggaccagcgactgcagctgagtcatgatctgctcggaatcctcctgctaaagaaggctctgggcgtgagcctcagccgccccgcaccccaaatccagtacaggaggctgctggtacaaatactgcagaaatgacaccaataatggggcagacacaacagcgtggcttagattgtgcccacccagggaaggtgctgaatgggaccctgttgatggccccatctggatgtaaatcctgagctcaaatctctgttactccattactgtgatttctggctgggtcaccagaaatatcgctgatgcagacacagattatgttcctgctgtatttcctgcttccctgttgaattggtgaataaaaccttgctctt

SEQ ID NO: 23 - DNA, 639 bp, Homo sapiens
gcgcgcccgaacgaagccgcggcccgggcacagccatggcccggcgggcggggggcgctcggatgttcggcagcctcctgctcttcgccctgctcgctgccggcgtcgccccgctcagctgggatctcccggagccccgcagccgagccagcaagatccgagtgcactcgcgaggcaacctctgggccaccggtcacttcatgggcaagaagagtctggagccttccagcccatcccattggggacagctccccacacctcccctgagggaccagcgactgcagctgagtcatgatctgctcggaatcctcctgctaaagaaggctctgggcgtgagcctcagccgccccgcaccccaaatccagtacaggaggctgctggtacaaatactgcagaaatgacaccaataataggggcagacacaacagcgtggcttagattgtgcccacccagggaaggtgctgaatgggaccctgttgatggccccatctggatgtaaatcctgagctcaaatctctgttactccattactgtgatttctggctgggtcaccagaaatatcgctgatgcagacacagattatgttcctgctgtatttcctgcttccctgttgaattggtgaataaaaccttgctcttt

SEQ ID NO: 24 - protein, 118 aa, Homo sapiens
RREGARMFGSLLHFALLAAGVVPLSWDLPEPRSRASKIRVHSRGKLWAIGHFMGKKSLEPSSPSPLGTAPHTSLRDQRLQLSHDLLGILLLKKALGVSLSRPAPQIQYRRLLVQILQK

SEQ ID NO: 25 - protein, 105 aa, Homo sapiens
RGARMFGSLLFALLAAGVPLSWDLPEPRSRASKIRVHSRGLWAGHFMGKKSLEPSSPSGPLRDQRLQLSHDLLGILLLKKALGVSLSRPAPQIQYRRLLVQILQK

SEQ ID NO: 26 - protein, 118 aa, Homo sapiens
RAGGARMFGSLLLFALLAAGVAPLSWDLPEPRSRASKIRVHSRGNLWATGHFMGKKSLEPSSPSHWGQLPTPPLRDQRLQLSHDLLGILLLKKALGVSLSRPAPQIQYRRLLVQILQK

SEQ ID NO: 27 - protein, 112 aa, Homo sapiens; identical to SEQ ID NO: 4

SEQ ID NO: 28 - protein, 117 aa, Homo sapiens
MTRQAGSTWLLRGLLLFALFVSGITPFSWDLPEPRSRASKIRVHPRGNLWATGHFMGKKSLEPPSLSLVGTAPPITQREQRLQLSHDLLRILLLQKALGMNLSGPAPPIQYRRLLQK

SEQ ID NO: 29 - protein, 121 aa, Homo sapiens
MARRAGGARMFGSLLLFALLAAGVAPLSWDLPEPRSRASKIRVHSRGNLWATGHFMGKKSLEPSSPSHWGQLPTPPLRDQRLQLSHDLLGILLLKKALGVSLSRPAPQIQYRRLLVQILQK

SEQ ID NO: 30 - DNA, 205 bp, Salmo salar
ctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagctg

SEQ ID NO: 31 - DNA, 124 bp, Artificial Sequence
  Description of Artificial Sequence consensus sequence
ctgcgggcctggaccggagcctttaacccatttactcacgttgacctacgagccagactcctccactgtccccggtgacccttcacctaccgtggctgctgtgactgcgctgcagcaccgactg

SEQ ID NO: 32 - DNA, 201 bp, Homo sapiens
ctgcagtggccactgcgtcaccaaggagccggttttcaagagcccattttccaccgtgtaccagcatgtgtgcacctaccgggacgtccgctatgaaacgatccgcctacctgactgtcccccttgggtggaccatcatgtcacctaccctgtggctctgagctgtgactgcagcctctgtaacatggacacttctgactg

SEQ ID NO: 33 - protein, 85 aa, Cyprinus carpio
TFLAKKPGCRGLRITTDACWGRCETWEKPILEPPYIEAHHRVCTYNETKQVTVKLPNCAPGVDPFYTYPVAIRCDCGACSTATTE

SEQ ID NO: 34 - protein, 37 aa, Artificial Sequence
  Description of Artificial Sequence consensus sequence
TKGCLTCGCTEPPVCTYTVLPCPGVDPTYPVACDCCT

SEQ ID NO: 35 - protein, 85 aa, Homo sapiens
TVAVEKEGCPKCLVLQTTICSGHCLTKEPVYKSPFSTVYQHVCTYRDVRYETVRLPDCPPGVDPHITYPVALSCDCSLCTMDTSD

SEQ ID NO: 36 - protein, 117 aa, Clupea pallasi
PMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTE

SEQ ID NO: 37 - protein, 47 aa, Artificial Sequence
  Description of Artificial Sequence consensus sequence
PLLCVLANLCTKGCRLTCGCTEPPVCTYTLPCAGVDPTYPVACCCST

SEQ ID NO: 38 - protein, 116 aa, Homo sapiens
PECTILLLLCMCVLAVPAQCFNLQPCVLVNETVSVEKEGCPRCLVFRTTICSGHCPTKEPVYKSPFSVVNQHVCTYGNFRYETIRLPDCADGVDPLVTYPVALSCECSLCSMDTSD

SEQ ID NO: 39 - protein, 101 aa, Homo sapiens
SSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTE

SEQ ID NO: 40 - protein, 40 aa, Homo sapiens
SGLRCTAKCTTCGCPPPRVCTYEVLPCPGVDPPVACCGCT

SEQ ID NO: 41 - protein, 99 aa, Homo sapiens
SRGPLRPLCQPINATLAAEKEACPVCITFTTSICAGYCPSMKRVLPVILPPMPQRVCTYHELRFASVRLPGCPPGVDPMVSFPVALSCHCGPCRLSSTD

SEQ ID NO: 42 - protein, 116 aa, Equus caballus
MALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTE

SEQ ID NO: 43 - protein, 43 aa, Artificial Sequence
  Description of Artificial Sequence consensus sequence
LLGVASGLRCTAKCTTCGCPAVCTYELPCPGVDPPVACCGCTT

SEQ ID NO: 44 - protein, 113 aa, Homo sapiens
LLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTD

SEQ ID NO: 45 - protein, 144 aa, Homo sapiens
MGTPVKILVVRNHILFSVVVLLAVAQSSYLPPCEPVNETVAVEKEGCPKCLVLQTTICSGHCLTKEPVYKSPFSTVYQHVCTYRDVRYETVRLPDCPPGVDPHITYPVALSCDCSLCTMDTSDCTIESLQPDFCMSQREDFLVY

SEQ ID NO: 46 - protein, 140 aa, Carassius auratus
MGTPVKILVVLFSVIVLLAVAQSSYLPPCEPVNETVAVEKEGCPKCLVLQTTICSGHCLTKEPVYKSPFSTVYQHVCTYRDVRYETVRLPDCPPGVDPHITYPVALSCDCSLCTMDTSDCTIESLQPDFCMSQREDFLVY

SEQ ID NO: 47 - protein, 141 aa, Bos taurus
MEMFQGLLLWLLLGVAGVWASRGPLRPLCQPINATLAAEKEACPVCITFTTSICAGYCPSMKRVLPVILPPMPQRVCTYHELRFASVRLPGCPPGVDPMVSFPVALSCHCGPCRLSSTDCGGPRTQPLACDHPPLPDILFL

SEQ ID NO: 48 - protein, 141 aa, Ovis aries
MEMLQGLLLWLLLGVAGVWASRGPLRPLCQPINATLAAEKEACPVCITFTTSICAGYCLSMKRVLPVILPPMPQRVCTYHELRFASVRLPGCPPGVDPMVSFPVALSCHCGPCRLSSTDCGGPRTQPLACDHPPLPDILFL

SEQ ID NO: 49 - protein, 230 aa, Homo sapiens; identical to SEQ ID NO: 6

SEQ ID NO: 50 - DNA, 215 bp, Equus caballus
aggatgtgaacattgaggaactgtacaaaggtggtgaagaggccacacgcttcaccttcttccagagcagctcaggctccgccttcaggcttgaggctgctgcctggcctggctggttcctgtgtggcccggcagagccccagcagccagtacagctcaccaaggagagtgagccctcagcccgtaccaagttttactttgaacagagctggtag

SEQ ID NO: 51 - DNA, 147 bp, Artificial Sequence
  Description of Artificial Sequence consensus sequence
agggtaacatgactgcaaaggagagcacgcttcaccttctccgcacggccccccagcttgagctgcgcctgcctggctggttccttgcgcagagcacgccgtcagctcaccaaagagagcctcagtaccaagtttacttaagcgtag

SEQ ID NO: 52 - DNA, 218 bp, Homo sapiens
aggcagttaacatcactgacctgagcaagaacaaggaggagaacaagcgcttcaccttcatccgctcaaacagtggccccaccaccagcttcgagtctgccgcctgccctggctggttcctctgcacggcgcaggaggcagaccggcccgtcagcctcaccaacaagcccaaagagtccttcatggtcaccaagttctacttccaggaggaccagtag

SEQ ID NO: 53 - protein, 149 aa, Mus musculus
CFRIKYADQKALYTRDGQLLVGDPVADNCCAEKICILPNRGLARTKVPIFLGIQGGSRCLACVETEEGPSLQLEPSTLPPQDVNIEELYKGGEEATRFTFFQSSSGSAFRLEAAAWPGWFLCGPAEPQQPVQLTKESEPSARTKFYFEQ

SEQ ID NO: 54 - protein, 70 aa, Artificial Sequence
  Description of Artificial Sequence consensus sequence
CFRKKLYQLLGAEIPNRLPLGQGGSCLCTEGPLLEPVNIELYGEFTFGEAAPGWFLCEQPVLTEATFYFQ

SEQ ID NO: 55 - protein, 146 aa, Homo sapiens
CFRMKDSALKVLYLHNNQLLAGGLHAEKVIKGEEISVVPNRALDASLSPVILGVQGGSQCLSCGTEKGPILKLEPVNIMELYLGAKESKSFTFYRRDMGLTSSFESAAYPGWFLCTSPEADQPVRLTQIPEDPAWDAPITDFYFQQ

SEQ ID NO: 56 - protein, 149 aa, Homo sapiens; identical to SEQ ID NO: 53

SEQ ID NO: 57 - protein, 67 aa, Homo sapiens
CFRKKLYQLLGAEIPNRLPLGQGGSCLCEPLLEPVNIELYGEFTFGEAAPGWFLCEQPVLTETFYFQ

SEQ ID NO: 58 - protein, 146 aa, Homo sapiens
CFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQ

SEQ ID NO: 59 - protein, 173 aa, Homo sapiens
DNHTMRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 60 - protein, 212 aa, Homo sapiens [sequence omitted]

SEQ ID NO: 61 - protein, 155 aa, Homo sapiens
MVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 62 - protein, 180 aa, Homo sapiens
MALADLYEEGGGGGGEGEDNADSKETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 63 - protein, 158 aa, Homo sapiens
GPSALSYCFRIKYADQKALYTRDGQLLVGDPVADNCCAEKICILPNRGLARTKVPIFLGIQGGSRCLACVETEEGPSLQLEPSTLPPQDVNIEELYKGGEEATRFTFFQSSSGSAFRLEAAAWPGWFLCGPAEPQQPVQLTKESEPSARTKFYFEQSW

SEQ ID NO: 64 - protein, 266 aa, Sus scrofa
MATVPEPINEVMAYYSDENELLFEVDGPKQMKSCTQHLDLGSMGDGNIQLQISHQLYNKSFRQVVSVIVAMEKLRSRAYEHVFRDDDLRSILSFIFEEEPVIFETSSDELLCDAAVQSVKCKLQDREQKSLVLDSPCVLKALHLLSQEMSREVVFCMSFVQGEERDNKIPVALGIRDKNLYLSCVKKGDTPTLQLEEVDPKVYPKRNMEKRFVFYKTEIKNTVEFESVLYPNWYISTSQIEEKPVFLGRFRGGQDITDFRMETLSP

SEQ ID NO: 65 - DNA, 329 bp, Sus scrofa
catttaatagcctgtagagacacagaattcagtgacaaggaaaagggtaatatggtttacctgggaatcaagggaaaagatctctgtctcttctgtgcagaaattcagggcaagcctactttgcagcttaaggaaaaaaatatcatggacctgtatgtggagaagaaagcacagaagccctttctctttttccacaataaagaaggctccacttctgtctttcagtcagtctcttaccctggctggttcatagccacctccaccacatcaggacagcccatctttctcaccaaggagagaggcataactaataacactaacttctactt

SEQ ID NO: 66 - DNA, 197 bp, Artificial Sequence
  Description of Artificial Sequence consensus sequence
catatactgagagaaagatgtgcgagtattgttcctgggatcaggaacttgcttctgtgaatcggagtatcagtagaaaatcagacctgagaagagcaaaagccttctttcccaagggcccaccctttagtcagcctcctggctggttctcactcacaacagcagcctctcaccaagagcataacacaattctactt

SEQ ID NO: 67 - DNA, 331 bp, Homo sapiens
caaatactaaactggaagagaagatagatgtggtgcctgttgagcctcattttgtgttcctggggatccatggagggaagctgtgcctgtcctgtgtcaagtctggtgatgagatgaagctccagttggacgcagttaacatcacagacctgagaaagaacagcgagcaggacaagcgcttcaccttcatccgctccgacagtggccccaccaccagctttgagtcagccgcctgtcctggctggttcctctgcactgcactagaggcagaccagcctgttggcctcaccaacacgcccaaagcagccgtcaaggtcaccaagttctactt

SEQ ID NO: 68 - protein, 149 aa, Homo sapiens
PKSYAIRDSRQMVWVLSGNSLIAAPLSRSIKPVTLHLIACRDTEFSDKEKGNMVYLGIKGKDLCLFCAEIQGKPTLQLKEKNIMDLYVEKKAQKPFLFFHNKEGSTSVFQSVSYPGWFIATSTTSGQPIFLTKERGITNNTNFYLDSVE

SEQ ID NO: 69 - protein, 149 aa, Homo sapiens; identical to SEQ ID NO: 68

SEQ ID NO: 70 - protein, 149 aa, Homo sapiens; identical to SEQ ID NO: 68

SEQ ID NO: 71 - protein, 85 aa, Homo sapiens
PKSYAIRDSRQMVWVLSGNSLIAAPLSRSIKPVTLHLIACRDTEFSDKEKGNMVYLGIKGKDLCLFCAEIQGKPTLQLKEKNIMD

SEQ ID NO: 72 - protein, 80 aa, Homo sapiens
PKSYAIRDSRQMVWVLSGNSLIAAPLSRSIKPVTLHLIACRDTEFSDKEKGNMVYLGIKGKDLCLFCAEIQGKPTLQLKD

SEQ ID NO: 73 - protein, 85 aa, Homo sapiens
PKSYAIRDSRQMVWVLSGNSLIAAPLSRSIKPVTLHLIACRDTEFSDKEKGNMVYLGIKGKDLCLFCAEIQGKPTLQLKLQGSQD

SEQ ID NO: 74 - protein, 146 aa, Homo sapiens
YAIRDSRQMVWVLSGNSLIAAPLSRSIKPVTLHLIACRDTEFSDKEKGNMVYLGIKGKDLCLFCAEIQGKPTLQLKEKNIMDLYVEKKAQKPFLFFHNKEGSTSVFQSVSYPGWFIATSTTSGQPIFLTKERGITNNTNFYLDSVE

SEQ ID NO: 75 - protein, 52 aa, Homo sapiens
DSVLNLALKIDLGGCLCQPTLLNIMLYKKFFGTSFSYPGWFTQPLTEGNTFY

SEQ ID NO: 76 - protein, 147 aa, Homo sapiens
FRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 77 - protein, 170 aa, Homo sapiens; identical to SEQ ID NO: 10

SEQ ID NO: 78 - protein, 212 aa, Homo sapiens [sequence omitted]

SEQ ID NO: 79 - protein, 180 aa, Homo sapiens; identical to SEQ ID NO: 62

SEQ ID NO: 80 - protein, 155 aa, Homo sapiens; identical to SEQ ID NO: 61

SEQ ID NO: 81 - protein, 266 aa, Homo sapiens; identical to SEQ ID NO: 64

SEQ ID NO: 82 - DNA, 244 bp, Homo sapiens
tctacctgggcctgaatggactcaatctctgcctgatgtgtgctaaagtcggggaccagcccacactgcagctgaagcttcaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttaccca

SEQ ID NO: 83 - DNA, 150 bp, Homo sapiens
ttcctgggtatggaaacttgcctgtgtgtaagtcgggaagactcagtgaccagaaatagatgaaaaaccgagcgaagcttcttcccccaagtggcacccacttgagtcggctcctggctggttctctgcgcctaggagcccttcctacca

SEQ ID NO: 84 - DNA, 238 bp, Homo sapiens
tgttcctggggatccatggagggaagctgtgcctgtcctgtgtcaagtctggtgatgagatgaagctccagttggacgcagttaacatcacagacctgagaaagaacagcgagcaggacaagcgcttcaccttcatccgctccgacagtggccccaccaccagctttgagtcagccgcctgtcctggctggttcctctgcactgcactagaggcagaccagcctgttggcctcaccaa

SEQ ID NO: 85 - protein, 130 aa, Homo sapiens; identical to SEQ ID NO: 12

SEQ ID NO: 86 - protein, 126 aa, Homo sapiens
DINHRVWVLQDQTLIAVPRKPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGK

SEQ ID NO: 87 - protein, 130 aa, Homo sapiens
DINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGK

SEQ ID NO: 88 - protein, 130 aa, Homo sapiens; identical to SEQ ID NO: 12

SEQ ID NO: 89 - protein, 82 aa, Homo sapiens
DNVWLQQLAVPRVPVTAICELERGPIYLGCLCKVGQPTLQLKEIMDLYQPEPVKFLFYGRSTESVAFPWFIASSPILTELGK

SEQ ID NO: 90 - protein, 129 aa, Homo sapiens
DLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGK

SEQ ID NO: 91 - protein, 81 aa, Mus musculus
IYLGLNGLNLCLMCAKVGDQPTLQLKLQEKDIMDLYNQPEPVKSFLFYHSQSGRNSTFESVAFPGWFIAVSSEGGCPLILT

SEQ ID NO: 92 - protein, 35 aa, Artificial Sequence
  Description of Artificial Sequence consensus sequence
LGGLCLCAKGDLLEIDLEKFFSGFESAPGWFEPLT

SEQ ID NO: 93 - protein, 79 aa, Homo sapiens
VFLGIHGGKLCLSCAKSGDDIKLQLEEVNITDLSKNKEEDKRFTFIRSEKGPTTSFESAACPGWFLCTTLEADRPVSLT

SEQ ID NO: 94 - protein, 178 aa, Mus musculus
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 95 - protein, 177 aa, Equus caballus
MEIRRRSVRHLISLLLFLFYSETACHPLGKRPCKMQAFRIWDVNQKTFYMRNNQLVAGYLQESNTKLQEKIDVVPIEPDALFLGLHGRKLCLACVKSGDEIRFQLEAVNITDLSKNKEENKRFTFIRSNSGPTTSFESAACPGWFLCTAQEADRPVSLTNKPKESFMVTKFYLQEDQ

SEQ ID NO: 96 - protein, 155 aa, Homo sapiens; identical to SEQ ID NO: 61

SEQ ID NO: 97 - protein, 130 aa, Homo sapiens; identical to SEQ ID NO: 12

SEQ ID NO: 98 - DNA, 20 bp, Artificial Sequence
  Description of Artificial Sequence  chemically synthesized
tgaagcttcagctgcagtgt

SEQ ID NO: 99 - DNA, 26 bp, Artificial Sequence
  Description of Artificial Sequence  chemically synthesized
ccgactttagcacacatcaggcagag

SEQ ID NO: 100 - DNA, 19 bp, Artificial Sequence
  Description of Artificial Sequence  chemically synthesized
gggcctgaatggactcaat